(19) United States
(12) Patent Application Publication
(10) Pub. No.: US 2022/0306677 A1
(43) Pub. Date: Sep. 29, 2022

(54) COMPOSITIONS AND METHODS FOR MAKING HYBRID POLYPEPTIDES

(71) Applicant: Yale University, New Haven, CT (US)

(72) Inventors: Omer Ad, New Haven, CT (US); Kyle S. Hoffman, New Haven, CT (US); Andrew G. Cairns, New Haven, CT (US); Aaron L. Featherston, New Haven, CT (US); Scott J. Miller, New Haven, CT (US); Dieter Soll, New Haven, CT (US); Alanna Schepartz, Berkeley, CA (US)

(21) Appl. No.: 17/616,948

(22) PCT Filed: Jun. 4, 2020
(86) PCT No.: PCT/US2020/036089

§ 371 Date: Dec. 6, 2021

Related U.S. Application Data
(60) Provisional application No. 62/857,184, filed on Jun. 4, 2019.

(51) Int. Cl. C07H 21/02; C12N 15/11

(52) U.S. Cl. CPC C07H 21/02; C12N 15/11; C07K 2319/00; C12N 2310/351

(57) ABSTRACT

Compositions and methods of making hybrid polypeptides and other polymers are disclosed. For example, functionalized tRNA having a functional molecule including a benzoic acid or benzoic acid derivative acylated to the 3′ nucleotide of a tRNA are provided. Functionalized tRNA having a functional molecule including a malonic acid or malonic acid derivative acylated to the 3′ nucleotide of a tRNA are also provided. Methods of using the functionalized tRNA for making compounds including the functional molecule are also provided. The methods typically include providing or expressing a messenger RNA (mRNA) encoding the target polypeptide in a translation system including one or more functionalized tRNA wherein each functionalized tRNA recognizes at least one codon such that its functional molecule is incorporated into the polypeptide or other polymer during translation. The incorporation of the functional molecule can occur in vitro in a cell-free translation system, or in vivo in a host cell.

### CROSS-REFERENCE TO RELATED APPLICATIONS

This application claims the benefit of and priority to U.S. Ser. No. 62/857,184 filed Jun. 4, 2019 and which is incorporated by reference in its entirety.

### STATEMENT REGARDING FEDERALLY SPONSORED RESEARCH

This invention was made with government support under 1740549 awarded by National Science Foundation. The government has certain rights in the invention.

### REFERENCE TO SEQUENCE LISTING

The Sequence Listing submitted as a text file named “YU_7718_PCT_ST25.txt,” created on Jun. 3, 2020, and having a size of 4,833 bytes is hereby incorporated by reference pursuant to 37 C.F.R § 1.52(e)(5).

### FIELD OF THE INVENTION

The field of the present invention generally relates to compositions and methods for incorporation non-amino acid function groups into polypeptides during translation.

### BACKGROUND OF THE INVENTION

Ribosomes have evolved for billions of years to perform a single reaction-formation of an amide bond between two α-amino acid substrates brought into proximity by tRNAs within the ribosome active site, the peptidyl transferase center (PTC). In cells and extracts, the chemistry possible within a wild type ribosome PTC has expanded to include reactions of more than 200 different non-proteinogenic α-amino- and hydroxy acids; (Guo et al., Chem. Int. Ed Engl. 47, 722-725 (2008), Chin, Nature 550, 53-60 (2017), Vargas-Rodriguez et al., Curr. Opin. Chem. Biol., 46, 115-122 (2018), Young et al., ACS Chem. Biol., 13, 854-870 (2018)) ribosomes containing remodeled PTCs support amide bond formation to and from a small number of β-amino acids (Maini et al., Bioorg. Med. Chem., 21, 1088-1096 (2013), Maini et al., Biochemistry, 54, 3694-3706 (2015), Melo Czekster et al., J. Am. Chem. Soc., 138, 5194-5197 (2016)) and dipeptides (Maini et al., J. Am. Chem. Soc., 137, 11206-11209 (2015), Chen et al., J. Am. Chem. Soc., 141, 5597-5601 (2019)) with limited efficiency. The combination of cell-free in vitro translation systems and ribozyme-catalyzed tRNA acylation reactions offers the opportunity for even greater reaction diversity, including the introduction of multiple N-alkyl, (Subtelny et al., J. Am. Chem. Soc., 130, 6131-6136 (2008)) D-α-, (Dedkova et al., J. Am. Chem. Soc., 125, 6616-6617(2003), Goto et al., RNA, 14, 1390-1398 (2008)) α-hydroxy, (Ohta et al., Chem. Biol., 14, 1315-1322 (2007)) and p-amino acids (Fujino et al., J. Am. Chem. Soc., 138, 1962-1969 (2016), Katoh & Suga, J. Am. Chem. Soc., 140, 12159-12167 (2018)). Recently, wild type E. coli ribosomes were shown to accept and elongate initiator tRNAs pre-charged with aromatic foldamer-dipeptide appendages (Rogers et al., Nat. Chem., 10, 405-412 (2018)). Notably, in this case the foldamer monomers did not themselves react within the PTC, being displaced from the reaction center by a Phe-Gly dipeptide spacer (Rogers et al., Nat. Chem., 10, 405-412 (2018), Schepartz, Nat. Chem., 10, 377-379 (2018)). Thus, there remains a need for improvement in methods of making hybrid polypeptide.

It is an object of the invention to provide compositions and methods of methods of making sequence defined hybrid polypeptides.

It is other object of the invention to provide compositions made according to the disclosed methods.

### SUMMARY OF THE INVENTION

Compositions and methods of making hybrid polypeptides are disclosed. For example, functionalized tRNA having a functional molecule including a benzoic acid or benzoic acid derivative acylated to the 3′ nucleotide of a tRNA are provided. Functionalized tRNA having a functional molecule including a malonic acid or malonic acid derivative acylated to the 3′ nucleotide of a tRNA are also provided. The tRNA can be any naturally occurring or non-naturally occurring tRNA or tRNA-like molecule. In some embodiments, the tRNA is from, or derived from, bacteria (e.g., E. coli), yeast, or humans. The tRNA can be an initiator tRNA or an elongator tRNA. In some embodiments, the tRNA is a suppressor tRNA.

Methods of using the functionalized tRNA for making sequence-defined functionalized polypeptides and polymers including one or more functional molecules are also provided. The methods typically include providing or expressing a messenger RNA (mRNA) encoding the target polypeptide in a translation system including one or more functionalized tRNA wherein each functionalized tRNA recognizes at least one codon such that its functional molecule can be incorporated into a polypeptide during translation. The incorporation of the functional molecule(s) can occur in vitro in a cell-free translation system, or in vivo in a host cell. In some embodiments, the host cell is a prokaryote, for example a bacteria such as E. coli. In some embodiments, one or more polynucleotides encoding the tRNA and a flexizyme or orthogonal amino acyl tRNA synthetase capable of acylating the tRNA with the functional molecule are expressed in the host cell.

Polypeptides and other sequence defined polymers having at least one functional molecule including a benzoic acid or benzoic acid derivative; or a malonic acid or malonic acid derivative are also provided. The polypeptides can by hybrid polypeptides that include a combination of functionalized molecules and amino acids. The functional molecule(s) can be positioned at the N-terminus, the C-terminus, internally within the polypeptide or polymer (i.e., not the N-terminus or C-terminus) or any combination thereof. When the polypeptide includes two or more functional molecules, the two or more functional molecules can be the same or different. In some embodiments, one or more of the functional molecule(s) do not include an amino acid.

### BRIEF DESCRIPTION OF THE DRAWINGS

FIG. 1A is a flow diagram illustrating a protocol used to detect acylation of microhelix (MH) or tRNA by cyanomethyl esters 1-3. FIG. 1B is a chart and photograph showing the results of an acid-urea gel-shift analysis of MH acylation by cyanomethyl esters 1-3 in the presence of ribozyme eFx. Yield was estimated by UV densitometry. LC-HRMS analysis of MH acylation reactions after RNase A digestion were separately investigated. Adenine nucleosides acylated on the 2′ or 3′ hydroxyl of the 3′ terminal ribose of MH could be detected in eFx-promoted reactions of the cyanomethyl ester of L-phenylalanine (Phe) and aminobenzoic acid esters 1 and 2; trace levels were detected in reactions containing 3. These products were not observed in analogous reactions containing m-aminobenzoic acid. FIG. 1C is a chart and photograph showing the results acid-urea gel-shift analysis of MH acylation by 1,3-dinitrobenzyl esters 4-5 in the presence of ribozyme dFx and by cyanomethyl ester 6 in the presence of eFx. Yield was estimated by UV densitometry. LC-HRMS analysis of MH acylation reactions after RNase A digestion were separately investigated. Adenine nucleosides acylated on the 2′ or 3′ hydroxyl of the 3′ terminal ribose could be detected only in the eFx-promoted reaction of cyanomethyl ester 6. FIG. 1D is a flow diagram of a protocol used to acylate tRNA using isatoic anhydride and analyze product formation. By LC-HRMS analysis of tRNA acylation reactions after RNase A digestion, adenine nucleosides acylated on the 2′ or 3′ hydroxyl of the 3′ terminal ribose could be detected in reactions of ValT and fMetT in the presence of isatoic anhydride and base, but not in their absence. Furthermore, tRNA acylation reactions using isatoic anhydride generate multiple products. ValT prepared by in vitro transcription migrates as a single peak when analyzed by UHPLC/UV, as does fMetT acylated with cyanomethyl ester 8. In contrast, the product of reaction of ValT with isatoic anhydride showed evidence for multiple products and/or degradation.

FIG. 2 is a flow diagram illustrating a protocol used to evaluate whether an initiator tRNA (fMetT) acylated with o-(prepared using isatoic anhydride) or m-aminobenzoic acid (prepared using eFx) (AN-tRNA) could support translation in vitro. LC-HRMS analysis of reaction products showing DNA template-dependent translation of a polypeptide whose mass corresponded to that of o-AN-VFDYKDDDDK (o-AN-VF-FLAG) (SEQ ID NO:16). No such polypeptide was observed in the absence of DNA template or in the presence of L-methionine. LC-HRMS analysis of an analogous β-Phe-containing polypeptide was also carried out and used for comparison purposes.

FIG. 3A is a chart and photograph showing the results of an acid-urea gel-shift analysis of MH acylation by cyanomethyl esters 6 and 8-15 in the presence of ribozyme eFx. Yield was estimated by UV densitometry. LC-HRMS analysis of MH acylation reactions containing cyanomethyl esters 6 and 8-15 after RNase A digestion was investigated separately. Exact masses are reported in Table 2. FIG. 3B is a chart and photograph showing the results of an acid-urea gel-shift analysis of MH acylation by cyanomethyl esters 16-18 in the presence of ribozyme eFx. Yield was estimated by UV densitometry. LC-HRMS analysis of MH acylation reactions containing cyanomethyl esters 16-18 after RNase A digestion was separately investigated. Evidence for acylation at the MH 3′-end is seen only in reactions containing cyanomethyl esters 17 and 18 but not 16. Exact masses are reported in Table 2.

FIGS. 4A and 4B are plots showing time-dependent synthesis of fMet-VF-FLAG in PURExpress® A reactions supplemented with 50 μM L-methionine (4A) or 50 μM FMetT-FMet (precharged with dFx) (4B). Product formation is represented by the abundance of the extracted ion 709.7927 m/z (M+2H). When initiated by addition of L-Methionine to PURExpress® Δ reactions, fMet-VF-FLAG is produced more rapidly, in higher yield, and over a longer time period than when fMet-VF-FLAG synthesis is initiated using pre-charged fMetT-fMet. FIG. 4C is a plot showing a time course of AR-VF-FLAG synthesis initiated using fMetT precharged with benzoic acid 8 (using eFx). FIG. 4D is a plot showing a time course of fMet-β-Phe-FV-FLAG synthesis initiated using 50 μM of ValT-β-Phe (using eFx). FIGS. 4E and 4F are bar graphs showing the relative yields of AR-VF-FLAG polypeptides produced after 6 h. Relative yield was calculated by dividing the extracted ion abundance of each AR-VF-FLAG polypeptide by the yield of a fMet-VF-FLAG from a reaction initiated with L-Methionine (330 μM) (for normalization). FIG. 4E illustrates a comparison between peptides initiated with aramid monomers and fMetT-fMet, while FIG. 4F illustrates a comparison among only the peptides initiated with aramid monomers.

FIG. 5 is a chart and photograph showing the results of acid-urea gel-shift analysis of MH acylation by malonic esters 19-23 in the presence of eFx (19, 23) or dFx (20-22). Yield was estimated by UV densitometry. LC-HRMS analysis of MH acylation reactions containing esters 19-23 after RNase A digestion was separately investigated. ND=Not determined due to lack of separation from unacylated microhelix. Exact masses are reported in Table 2.

FIG. 6A is a diagram illustrating an exemplary method of making aramid- and polyketide-peptide hybrid molecules (SEQ ID NO:17) using wildtype E. coli ribosomes and translation factors and tRNAs charged with aramid and polyketide moieties. FIG. 6B is a flow chart illustrating how flexizyme (SEQ ID NO:18, partial sequence) can charge the 3′ adenosine of a tRNA.

FIGS. 7A-7D are structures of oligomers prepared according to the disclosed methods. FIG. 7A illustrates a hybrid aramid-peptide molecule formed when p-amino benzoic acid-Phe double monomer (para-armamid-Phe) is loaded into the A site of a ribosome and added to the C-terminal end of a growing polypeptide during translation. FIG. 7B illustrates a hybrid aramid-peptide molecule formed when an o-amino benzoic acid monomer (ortho-aramid) is loaded into the P site of a ribosome by an initiator tRNA and forms the N-terminus of a growing polypeptide during translation. FIG. 7C illustrates a hybrid aramid-peptide molecule formed when an p-nitro benzoic acid monomer (p-nitro aramid) is loaded into the P site of a ribosome by an initiator tRNA and forms the N-terminus of a growing polypeptide during translation. FIG. 7D illustrates a hybrid ketide-peptide molecule formed when a substituted malonic acid monomer (p-nitro aramid) is loaded into the P site of a ribosome by an initiator tRNA and forms the N-terminus of a growing polypeptide during translation.

### DETAILED DESCRIPTION OF THE INVENTION

### I. Definitions

Transfer RNA or tRNA refers to a set of genetically encoded RNAs that act during protein synthesis as adaptor molecules, matching individual amino acids to their corresponding codon on a messenger RNA (mRNA). In higher eukaryotes such as mammals, there is at least one tRNA for each of the 20 naturally occurring amino acids. In eukaryotes, including mammals, tRNAs are encoded by families of genes that are 73 to 150 base pairs long. tRNAs assume a secondary structure with four base paired stems known as the cloverleaf structure. The tRNA contains a stem and an anticodon. The anticodon is complementary to the codon specifying the tRNA's corresponding amino acid. The anticodon is in the loop that is opposite of the stem containing the terminal nucleotides. The 3′ end of a tRNA is aminoacylated by a tRNA synthetase so that an amino acid is attached to the 3′end of the tRNA. This amino acid is delivered to a growing polypeptide chain as the anticodon sequence of the tRNA reads a codon triplet in an mRNA.

As used herein, an “anticodon” refers to a unit made up of any combination of 2, 3, 4, and 5 bases (G or A or U or C), typically three nucleotides, that correspond to the three bases of a codon on an mRNA. Each tRNA contains a specific anticodon triplet sequence that can base-pair to one or more codons for an amino acid or a “stop codon.” Known “stop codons” include, but are not limited to, the three codon bases, UAA known as ochre, UAG known as amber and UGA known as opal, that do not code for an amino acid but act as signals for the termination of protein synthesis. tRNAs do not decode stop codons naturally, but can and have been engineered to do so. Stop codons are usually recognized by enzymes (release factors) that cleave the polypeptide as opposed to encode an AA via a tRNA. Generally the anticodon loop consists of seven nucleotides. In the 5′ to 3′ direction the first two positions 32 and 33 precede the anticodon positions 34 to 36 followed by two nucleotides in positions 37 and 38 (Alberts, B., et al. in The Molecular Biology of the Cell, 4^{th} ed, Garland Science, New York, N.Y. (2002)). The size and nucleotide composition of the anticodon is generally the same as the size of the codon with complementary nucleotide composition. A four base pair codon consists of four bases such as 5′-AUGC-3′ and an anticodon for such a codon would complement the codon such that the tRNA contained 5′-GCAU-3′ with the anticodon starting at position 34 of the tRNA. A 5 base codon 5′-CGGUA-3′ codon is recognized by the 5′-UACCG-3′ anticodon (Hohsaka T., et al. Nucleic Acids Res. 29:3646-3651 (2001)). The composition of any such anticodon for 2 (16=any possible combination of 4 nucleotides), 3 (64), 4 (256), and 5 (1024) base codons would follow the same logical composition. The “anticodon” typically starts at position 34 of a canonical tRNA, but may also reside in any position of the “anti-codon stem-loop” such that the resulting tRNA is complementary to the “stop codon” of equivalent and complementary base composition.

As used herein “suppressor tRNA” refers to a tRNA that alters the reading of a messenger RNA (mRNA) in a given translation system. For example, a suppressor tRNA can read through a stop codon.

As used herein “aminoacyl-tRNA Synthetases” (AARS) are enzymes that charge (acylate) tRNAs with amino acids. These charged aminoacyl-tRNAs then participate in mRNA translation and protein synthesis. The AARS show high specificity for charging a specific tRNA with the appropriate amino acid, for example, tRNA^{Val} with valine by valyl-tRNA synthetase or tRNA^{Trp} with tryptophan by tryptophanyl-tRNA synthetase. In general, there is at least one AARS for each of the twenty amino acids.

As used herein “transgenic organism” as used herein, is any organism, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. Suitable transgenic organisms include, but are not limited to, bacteria, cyanobacteria, fungi, plants and animals. The nucleic acids described herein can be introduced into the host by methods known in the art, for example infection, transfection, transformation or transconjugation. Techniques for transferring DNA into such organisms are widely known and provided in references such as Sambrook, et al. (2000) Molecular Cloning: A Laboratory Manual, 3^{rd} ed., vol. 1-3, Cold Spring Harbor Press, Plainview N.Y.

As used herein, the term “eukaryote” or “eukaryotic” refers to organisms or cells or tissues derived therefrom belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

As used herein, the term “non-eukaryotic organism” refers to organisms including, but not limited to, organisms of the Eubacteria phylogenetic domain, such as Escherichia coli, Thermus thermophilus, and Bacillus stearothermophilus, or organisms of the Archaea phylogenetic domain such as, Methanocaldococcus jannaschii, Methanothermobacter thermautotrophicus, Halobacterium such as Haloferax volcanii and Halobacterium species NRC-1, Archaeoglobusfulgidus, Pyrococcus furiosus, Pyrococcus horikoshii, and Aeuropyrum pernix.

As used herein, the term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “orthologous genes” or “orthologs” refer to genes that have a similar nucleic acid sequence because they were separated by a speciation event.

As used herein, the terms “protein,” “polypeptide,” and “peptide” refers to a natural or synthetic molecule having two or more amino acids linked by the carboxyl group of one amino acid to the alpha amino group of another. The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus.

In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. For example, isolated nucleic acids or protein can be at least 60% free, preferably 75% free, and most preferably 90% free from other associated components.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more expression control sequences.

As used herein, the term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the term “transformed,” “transgenic,” “transfected” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

As used herein, the term “endogenous” with regard to a nucleic acid refers to nucleic acids normally present in the host.

As used herein, the term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “recoded organism” and “genomically recoded organism (GRO)” in the context of codons refer to an organism in which the genetic code of the organism has been altered such that a codon has been eliminated from the genetic code by reassignment to a synonymous or nonsynonymous codon.

As used herein, the term “translation system” refers to the components necessary to incorporate an amino acid into a growing polypeptide chain (protein). Components of a translation system generally include amino acids, ribosomes, tRNAs, AARS, mRNA, as well as initiation, elongation, and termination factors. The components described herein can be added to a translation system, in vivo or in vitro, to incorporate amino acids and functional molecules into a protein. A translation system can be prokaryotic, e.g., an E. coli cell, eukaryotic, e.g., a yeast, mammal, plant, or insect or cells thereof, or cell-free.

As used herein, “genetically modified organism (GMO)” refers to any organism whose genetic material has been modified (e.g., altered, supplemented, etc.) using genetic engineering techniques. The modification can be extrachromasomal (e.g., an episome, plasmid, etc.), by insertion or modification of the organism's genome, or a combination thereof.

As used herein, “standard amino acid” and “canonical amino acid” refer to the twenty alpha-(α) amino acids that are encoded directly by the codons of the universal genetic code denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “non-standard amino acid (nsAA)” refers to any and all amino acids that are not a standard amino acid. Non-standard amino acids include beta-(β-), gamma-(γ-) or delta-(δ-) amino acids, or derivatives of anthranilic acid, or dipeptide units containing any of these variants. nsAA can be created by enzymes through posttranslational modifications; or those that are not found in nature and are entirely synthetic (e.g., synthetic amino acids (sAA)). In both classes, the nsAAs can be made synthetically. Non-standard-, non-natural-, and non-α-amino acids are known in the art. For example, WO 2015/120287 provides a non-exhaustive list of exemplary non-standard and synthetic amino acids that are known in the art (see, e.g., Table 11 of WO 2015/120287).

As used herein, the term “alkyl” refers to univalent groups derived from alkanes by removal of a hydrogen atom from any carbon atom. Alkanes represent saturated hydrocarbons, including those that are cyclic (either monocyclic or polycyclic). Alkyl groups can be linear, branched, or cyclic. Preferred alkyl groups have one to 30 carbon atoms, i.e., C₁-C₃₀ alkyl. In some forms, a C₁-C₃₀ alkyl can be a linear C₁-C₃₀ alkyl, a branched C₁-C₃₀ alkyl, or a linear or branched C₁-C₃₀ alkyl. More preferred alkyl groups have one to 20 carbon atoms, i.e., C₁-C₂₀ alkyl. In some forms, a C₁-C₂₀ alkyl can be a linear C₁-C₂₀ alkyl, a branched C₁-C₂₀ alkyl, or a linear or branched C₁-C₂₀ alkyl. Still more preferred alkyl groups have one to 10 carbon atoms, i.e., C₁-C₂₀ alkyl. In some forms, a C₁-C₁₀ alkyl can be a linear C₁-C₁₀ alkyl, a branched C₁-C₁₀ alkyl, or a linear or branched C₁-C₁₀ alkyl. The most preferred alkyl groups have one to 6 carbon atoms, i.e., C₁-C₆ alkyl. In some forms, a C₁-C₆ alkyl can be a linear C₁-C₆ alkyl, a branched C₁-C₆ alkyl, or a linear or branched C₁-C₆ alkyl. Preferred C₁-C₆ alkyl groups have one to four carbons, i.e., C₁-C₄ alkyl. In some forms, a C₁-C₄ alkyl can be a linear C₁-C₄ alkyl, a branched C₁-C₄ alkyl, or a linear or branched C₁-C₄ alkyl. Any C₁-C₃₀ alkyl, C₁-C₂₀ alkyl, C₁-C₁₀ alkyl, C₁-C₆ alkyl, and/or C₁-C₄ alkyl groups can, alternatively, be cyclic. If the alkyl is branched, it is understood that at least four carbons are present. If the alkyl is cyclic, it is understood that at least three carbons are present.

As used herein, the term “heteroalkyl” refers to alkyl groups where one or more carbon atoms are replaced with a heteroatom, such as, O, N, or S. Heteroalkyl groups can be linear, branched, or cyclic. Preferred heteroalkyl groups have one to 30 carbon atoms, i.e., C₁-C₃₀ heteroalkyl. In some forms, a C₁-C₃₀ heteroalkyl can be a linear C₁-C₃₀ heteroalkyl, a branched C₁-C₃₀ heteroalkyl, or a linear or branched C₁-C₃₀ heteroalkyl. More preferred heteroalkyl groups have one to 20 carbon atoms, i.e., C₁-C₂₀ heteroalkyl. In some forms, a C₁-C₂₀ heteroalkyl can be a linear C₁-C₂₀ heteroalkyl, a branched C₁-C₂₀ heteroalkyl, or a linear or branched C₁-C₂₀ heteroalkyl. Still more preferred heteroalkyl groups have one to 10 carbon atoms, i.e., C₁-C₂₀ heteroalkyl. In some forms, a C₁-C₁₀ heteroalkyl can be a linear C₁-C₁₀ heteroalkyl, a branched C₁-C₁₀ heteroalkyl, or a linear or branched C₁-C₁₀ heteroalkyl. The most preferred heteroalkyl groups have one to 6 carbon atoms, i.e., C₁-C₆ heteroalkyl. In some forms, a C₁-C₆ heteroalkyl can be a linear C₁-C₆ heteroalkyl, a branched C₁-C₆ heteroalkyl, or a linear or branched C₁-C₆ heteroalkyl. Preferred C₁-C₆ heteroalkyl groups have one to four carbons, i.e., C₁-C₄ heteroalkyl. In some forms, a C₁-C₄ heteroalkyl can be a linear C₁-C₄ heteroalkyl, a branched C₁-C₄ heteroalkyl, or a linear or branched C₁-C₄ heteroalkyl. If the heteroalkyl is branched, it is understood that at least four carbons are present. If the heteroalkyl is cyclic, it is understood that at least three carbons are present.

As used herein, the term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear, branched, or cyclic. Preferred alkenyl groups have two to 30 carbon atoms, i.e., C₂-C₃₀ alkenyl. In some forms, a C₂-C₃₀ alkenyl can be a linear C₂-C₃₀ alkenyl, a branched C₂-C₃₀ alkenyl, a cyclic C₂-C₃₀ alkenyl, a linear or branched C₂-C₃₀ alkenyl, a linear or cyclic C₂-C₃₀ alkenyl, a branched or cyclic C₂-C₃₀ alkenyl, or a linear, branched, or cyclic C₂-C₃₀ alkenyl. More preferred alkenyl groups have two to 20 carbon atoms, i.e., C₂-C₂₀ alkenyl. In some forms, a C₂-C₂₀ alkenyl can be a linear C₂-C₂₀ alkenyl, a branched C₂-C₂₀ alkenyl, a cyclic C₂-C₂₀ alkenyl, a linear or branched C₂-C₂₀ alkenyl, a branched or cyclic C₂-C₂₀ alkenyl, or a linear, branched, or cyclic C₂-C₂₀ alkenyl. Still more preferred alkenyl groups have two to 10 carbon atoms, i.e., C₂-C₁₀ alkenyl. In some forms, a C₂-C₁₀ alkenyl can be a linear C₂-C₁₀ alkenyl, a branched C₂-C₁₀ alkenyl, a cyclic C₂-C₁₀ alkenyl, a linear or branched C₂-C₁₀ alkenyl, a branched or cyclic C₂-C₁₀ alkenyl, or a linear, branched, or cyclic C₂-C₂₀ alkenyl. The most preferred alkenyl groups have two to 6 carbon atoms, i.e., C₂-C₆ alkenyl. In some forms, a C₂-C₆ alkenyl can be a linear C₂-C₆ alkenyl, a branched C₂-C₆ alkenyl, a cyclic C₂-C₆ alkenyl, a linear or branched C₂-C₆ alkenyl, a branched or cyclic C₂-C₆ alkenyl, or a linear, branched, or cyclic C₂-C₆ alkenyl. Preferred C₂-C₆ alkenyl groups have two to four carbons, i.e., C₂-C₄ alkenyl. In some forms, a C₂-C₄ alkenyl can be a linear C₂-C₄ alkenyl, a branched C₂-C₄ alkenyl, a cyclic C₂-C₄ alkenyl, a linear or branched C₂-C₄ alkenyl, a branched or cyclic C₂-C₄ alkenyl, or a linear, branched, or cyclic C₂-C₄ alkenyl. If the alkenyl is branched, it is understood that at least four carbons are present. If the alkenyl is cyclic, it is understood that at least three carbons are present.

As used herein, the term “heteroalkenyl” refers to alkenyl groups in which one or more doubly bonded carbon atoms are replaced by a heteroatom. Heteroalkenyl groups can be linear, branched, or cyclic. Preferred heteroalkenyl groups have two to 30 carbon atoms, i.e., C₂-C₃₀ heteroalkenyl. In some forms, a C₂-C₃₀ heteroalkenyl can be a linear C₂-C₃₀ heteroalkenyl, a branched C₂-C₃₀ heteroalkenyl, a cyclic C₂-C₃₀ heteroalkenyl, a linear or branched C₂-C₃₀ heteroalkenyl, a linear or cyclic C₂-C₃₀ heteroalkenyl, a branched or cyclic C₂-C₃₀ heteroalkenyl, or a linear, branched, or cyclic C₂-C₃₀ heteroalkenyl. More preferred heteroalkenyl groups have two to 20 carbon atoms, i.e., C₂-C₂₀ heteroalkenyl. In some forms, a C₂-C₂₀ heteroalkenyl can be a linear C₂-C₂₀ heteroalkenyl, a branched C₂-C₂₀ heteroalkenyl, a cyclic C₂-C₂₀ heteroalkenyl, a linear or branched C₂-C₂₀ heteroalkenyl, a branched or cyclic C₂-C₂₀ heteroalkenyl, or a linear, branched, or cyclic C₂-C₂₀ heteroalkenyl. Still more preferred heteroalkenyl groups have two to 10 carbon atoms, i.e., C₂-C₁₀ heteroalkenyl. In some forms, a C₂-C₁₀ heteroalkenyl can be a linear C₂-C₁₀ heteroalkenyl, a branched C₂-C₁₀ heteroalkenyl, a cyclic C₂-C₁₀ heteroalkenyl, a linear or branched C₂-C₁₀ heteroalkenyl, a branched or cyclic C₂-C₁₀ heteroalkenyl, or a linear, branched, or cyclic C₂-C₂₀ heteroalkenyl. The most preferred heteroalkenyl groups have two to 6 carbon atoms, i.e., C₂-C₆ heteroalkenyl. In some forms, a C₂-C₆ heteroalkenyl can be a linear C₂-C₆ heteroalkenyl, a branched C₂-C₆ heteroalkenyl, a cyclic C₂-C₆ heteroalkenyl, a linear or branched C₂-C₆ heteroalkenyl, a branched or cyclic C₂-C₆ heteroalkenyl, or a linear, branched, or cyclic C₂-C₆ heteroalkenyl. Preferred C₂-C₆ heteroalkenyl groups have two to four carbons, i.e., C₂-C₄ heteroalkenyl. In some forms, a C₂-C₄ heteroalkenyl can be a linear C₂-C₄ heteroalkenyl, a branched C₂-C₄ heteroalkenyl, a cyclic C₂-C₄ heteroalkenyl, a linear or branched C₂-C₄ heteroalkenyl, a branched or cyclic C₂-C₄ heteroalkenyl, or a linear, branched, or cyclic C₂-C₄ heteroalkenyl. If the heteroalkenyl is branched, it is understood that at least four carbons are present. If heteroalkenyl is cyclic, it is understood that at least three carbons are present.

As used herein, the term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear, branched, or cyclic. Preferred alkynyl groups have two to 30 carbon atoms, i.e., C₂-C₃₀ alkynyl. In some forms, a C₂-C₃₀ alkynyl can be a linear C₂-C₃₀ alkynyl, a branched C₂-C₃₀ alkynyl, a cyclic C₂-C₃₀ alkynyl, a linear or branched C₂-C₃₀ alkynyl, a linear or cyclic C₂-C₃₀ alkynyl, a branched or cyclic C₂-C₃₀ alkynyl, or a linear, branched, or cyclic C₂-C₃₀ alkynyl. More preferred alkynyl groups have two to 20 carbon atoms, i.e., C₂-C₂₀ alkynyl. In some forms, a C₂-C₂₀ alkynyl can be a linear C₂-C₂₀ alkynyl, a branched C₂-C₂₀ alkynyl, a cyclic C₂-C₂₀ alkynyl, a linear or branched C₂-C₂₀ alkynyl, a branched or cyclic C₂-C₂₀ alkynyl, or a linear, branched, or cyclic C₂-C₂₀ alkynyl. Still more preferred alkynyl groups have two to 10 carbon atoms, i.e., C₂-C₁₀ alkynyl. In some forms, a C₂-C₁₀ alkynyl can be a linear C₂-C₁₀ alkynyl, a branched C₂-C₁₀ alkynyl, a cyclic C₂-C₁₀ alkynyl, a linear or branched C₂-C₁₀ alkynyl, a branched or cyclic C₂-C₁₀ alkynyl, or a linear, branched, or cyclic C₂-C₂₀ alkynyl. The most preferred alkynyl groups have two to 6 carbon atoms, i.e., C₂-C₆ alkynyl. In some forms, a C₂-C₆ alkynyl can be a linear C₂-C₆ alkynyl, a branched C₂-C₆ alkynyl, a cyclic C₂-C₆ alkynyl, a linear or branched C₂-C₆ alkynyl, a branched or cyclic C₂-C₆ alkynyl, or a linear, branched, or cyclic C₂-C₆ alkynyl. Preferred C₂-C₆ alkynyl groups have two to four carbons, i.e., C₂-C₄ alkynyl. In some forms, a C₂-C₄ alkynyl can be a linear C₂-C₄ alkynyl, a branched C₂-C₄ alkynyl, a cyclic C₂-C₄ alkynyl, a linear or branched C₂-C₄ alkynyl, a branched or cyclic C₂-C₄ alkynyl, or a linear, branched, or cyclic C₂-C₄ alkynyl. If the alkynyl is branched, it is understood that at least four carbons are present. If alkynyl is cyclic, it is understood that at least three carbons are present.

As used herein, the term “heteroalkynyl” refers to alkynyl groups in which one or more triply bonded carbon atoms are replaced by a heteroatom. Heteroalkynyl groups can be linear, branched, or cyclic. Preferred heteroalkynyl groups have two to 30 carbon atoms, i.e., C₂-C₃₀ heteroalkynyl. In some forms, a C₂-C₃₀ heteroalkynyl can be a linear C₂-C₃₀ heteroalkynyl, a branched C₂-C₃₀ heteroalkynyl, a cyclic C₂-C₃₀ heteroalkynyl, a linear or branched C₂-C₃₀ heteroalkynyl, a linear or cyclic C₂-C₃₀ heteroalkynyl, a branched or cyclic C₂-C₃₀ heteroalkynyl, or a linear, branched, or cyclic C₂-C₃₀ heteroalkynyl. More preferred heteroalkynyl groups have two to 20 carbon atoms, i.e., C₂-C₂₀ heteroalkynyl. In some forms, a C₂-C₂₀ heteroalkynyl can be a linear C₂-C₂₀ heteroalkynyl, a branched C₂-C₂₀ heteroalkynyl, a cyclic C₂-C₂₀ heteroalkynyl, a linear or branched C₂-C₂₀ heteroalkynyl, a branched or cyclic C₂-C₂₀ heteroalkynyl, or a linear, branched, or cyclic C₂-C₂₀ heteroalkynyl. Still more preferred heteroalkynyl groups have two to 10 carbon atoms, i.e., C₂-C₁₀ heteroalkynyl. In some forms, a C₂-C₁₀ heteroalkynyl can be a linear C₂-C₁₀ heteroalkynyl, a branched C₂-C₁₀ heteroalkynyl, a cyclic C₂-C₁₀ heteroalkynyl, a linear or branched C₂-C₁₀ heteroalkynyl, a branched or cyclic C₂-C₁₀ heteroalkynyl, or a linear, branched, or cyclic C₂-C₂₀ heteroalkynyl. The most preferred heteroalkynyl groups have two to 6 carbon atoms, i.e., C₂-C₆ heteroalkynyl. In some forms, a C₂-C₆ heteroalkynyl can be a linear C₂-C₆ heteroalkynyl, a branched C₂-C₆ heteroalkynyl, a cyclic C₂-C₆ heteroalkynyl, a linear or branched C₂-C₆ heteroalkynyl, a branched or cyclic C₂-C₆ heteroalkynyl, or a linear, branched, or cyclic C₂-C₆ heteroalkynyl. Preferred C₂-C₆ heteroalkynyl groups have two to four carbons, i.e., C₂-C₄ heteroalkynyl. In some forms, a C₂-C₄ heteroalkynyl can be a linear C₂-C₄ heteroalkynyl, a branched C₂-C₄ heteroalkynyl, a cyclic C₂-C₄ heteroalkynyl, a linear or branched C₂-C₄ heteroalkynyl, a branched or cyclic C₂-C₄ heteroalkynyl, or a linear, branched, or cyclic C₂-C₄ heteroalkynyl. If the heteroalkynyl is branched, it is understood that at least four carbons are present. If heteroalkynyl is cyclic, it is understood that at least three carbons are present.

As used herein, the term “aryl” refers to univalent groups derived from arenes by removal of a hydrogen atom from a ring atom. Arenes are monocyclic and polycyclic aromatic hydrocarbons. In polycyclic aryl groups, the rings can be attached together in a pendant manner or can be fused. Preferred aryl groups have six to 50 carbon atoms, i.e., C₆-C₅₀ aryl. In some forms, a C₆-C₅₀ aryl can be a branched C₆-C₅₀ aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl. More preferred aryl groups have six to 30 carbon atoms, i.e., C₆-C₃₀ aryl. In some forms, a C₆-C₃₀ aryl can be a branched C₆-C₃₀ aryl, a monocyclic C₆-C₃₀ aryl, a polycyclic C₆-C₃₀ aryl, a branched polycyclic C₆-C₃₀ aryl, a fused polycyclic C₆-C₃₀ aryl, or a branched fused polycyclic C₆-C₃₀ aryl. Even more preferred aryl groups have six to 20 carbon atoms, i.e., C₆-C₂₀ aryl. In some forms, a C₆-C₂₀ aryl can be a branched C₆-C₂₀ aryl, a monocyclic C₆-C₂₀ aryl, a polycyclic C₆-C₂₀ aryl, a branched polycyclic C₆-C₂₀ aryl, a fused polycyclic C₆-C₂₀ aryl, or a branched fused polycyclic C₆-C₂₀ aryl. The most preferred aryl groups have six to twelve carbon atoms, i.e., C₆-C₁₂ aryl. In some forms, a C₆-C₁₂ aryl can be a branched C₆-C₁₂ aryl, a monocyclic C₆-C₁₂ aryl, a polycyclic C₆-C₁₂ aryl, a branched polycyclic C₆-C₁₂ aryl, a fused polycyclic C₆-C₁₂ aryl, or a branched fused polycyclic C₆-C₁₂ aryl. Preferred C₆-C₁₂ aryl groups have six to eleven carbon atoms, i.e., C₆-C₁₁ aryl. In some forms, a C₆-C₁₁ aryl can be a branched C₆-C₁₁ aryl, a monocyclic C₆-C₁₁ aryl, a polycyclic C₆-C₁₁ aryl, a branched polycyclic C₆-C₁₁ aryl, a fused polycyclic C₆-C₁₁ aryl, or a branched fused polycyclic C₆-C₁₁ aryl. More preferred C₆-C₁₂ aryl groups have six to nine carbon atoms, i.e., C₆-C₉ aryl. In some forms, a C₆-C₉ aryl can be a branched C₆-C₉ aryl, a monocyclic C₆-C₉ aryl, a polycyclic C₆-C₉ aryl, a branched polycyclic C₆-C₉ aryl, a fused polycyclic C₆-C₉ aryl, or a branched fused polycyclic C₆-C₉ aryl. The most preferred C₆-C₁₂ aryl groups have six carbon atoms, i.e., C₆ aryl. In some forms, a C₆ aryl can be a branched C₆ aryl or a monocyclic C₆ aryl.

As used herein, the term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). In polycyclic heteroaryl groups, the rings can be attached together in a pendant manner or can be fused. Preferred heteroaryl groups have three to 50 carbon atoms, i.e., C₃-C₅₀ heteroaryl. In some forms, a C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl. More preferred heteroaryl groups have six to carbon atoms, i.e., C₆-C₃₀ heteroaryl. In some forms, a C₆-C₃₀ heteroaryl can be a branched C₆-C₃₀ heteroaryl, a monocyclic C₆-C₃₀ heteroaryl, a polycyclic C₆-C₃₀ heteroaryl, a branched polycyclic C₆-C₃₀ heteroaryl, a fused polycyclic C₆-C₃₀ heteroaryl, or a branched fused polycyclic C₆-C₃₀ heteroaryl. Even more preferred heteroaryl groups have six to 20 carbon atoms, i.e., C₆-C₂₀ heteroaryl. In some forms, a C₆-C₂₀ heteroaryl can be a branched C₆-C₂₀ heteroaryl, a monocyclic C₆-C₂₀ heteroaryl, a polycyclic C₆-C₂₀ heteroaryl, a branched polycyclic C₆-C₂₀ heteroaryl, a fused polycyclic C₆-C₂₀ heteroaryl, or a branched fused polycyclic C₆-C₂₀ heteroaryl. The most preferred heteroaryl groups have six to twelve carbon atoms, i.e., C₆-C₁₂ heteroaryl. In some forms, a C₆-C₁₂ heteroaryl can be a branched C₆-C₁₂ heteroaryl, a monocyclic C₆-C₁₂ heteroaryl, a polycyclic C₆-C₁₂ heteroaryl, a branched polycyclic C₆-C₁₂ heteroaryl, a fused polycyclic C₆-C₁₂ heteroaryl, or a branched fused polycyclic C₆-C₁₂ heteroaryl. Preferred C₆-C₁₂ heteroaryl groups have six to eleven carbon atoms, i.e., C₆-C₁n heteroaryl. In some forms, a C₆-C₁₁ heteroaryl can be a branched C₆-C₁₁ heteroaryl, a monocyclic C₆-C₁₁ heteroaryl, a polycyclic C₆-C₁₁ heteroaryl, a branched polycyclic C₆-C₁₁ heteroaryl, a fused polycyclic C₆-C₁₁ heteroaryl, or a branched fused polycyclic C₆-C₁₁ heteroaryl. More preferred C₆-C₁₂ heteroaryl groups have six to nine carbon atoms, i.e., C₆-C₉ heteroaryl. In some forms, a C₆-C₉ heteroaryl can be a branched C₆-C₉ heteroaryl, a monocyclic C₆-C₉ heteroaryl, a polycyclic C₆-C₉ heteroaryl, a branched polycyclic C₆-C₉ heteroaryl, a fused polycyclic C₆-C₉ heteroaryl, or a branched fused polycyclic C₆-C₉ heteroaryl. The most preferred C₆-C₁₂ heteroaryl groups have six carbon atoms, i.e., C₆ heteroaryl. In some forms, a C₆ heteroaryl can be a branched C₆ heteroaryl, a monocyclic C₆ heteroaryl, a polycyclic C₆ heteroaryl, a branched polycyclic C₆ heteroaryl, a fused polycyclic C₆ heteroaryl, or a branched fused polycyclic C₆ heteroaryl.

As used herein, the term “hydroxamate” refers to —C(═O)NH—OH, where the hydrogen atoms can be substituted with substituents.

As used herein, the term “derivative” as relates to a given compound or moiety, refers to another compound or moiety that is structurally similar, functionally similar, or both, to the specified compound or moiety. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered a derivative of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified, if it induces the same effect as the specified compound. “Derivative” can also refer to a modification including, but not limited to, hydrolysis, reduction, or oxidation products, of the compound or moiety. Hydrolysis, reduction, and oxidation reactions are known in the art.

As used herein, the term “substituted,” means that the chemical group or moiety contains one or more substituents replacing the hydrogen atoms in the chemical group or moiety. The substituents include, but are not limited to:
a halogen atom, an alkyl group, a cycloalkyl group, a heteroalkyl group, a cycloheteroalkyl group, an alkenyl group, a heteroalkenyl group, an alkynyl group, a heteroalkynyl group, an aryl group, a heteroaryl group, a polyaryl group, a polyheteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{T1′})(OR^{T2′}), —OP(═O)(OR^{T1′})(OR^{T2′}), —BR^{T1′}(OR^{T2′}), —B(OR^{T1′})(OR^{T2′}), or -G′R^{T1′} in which -T′ is —O—, —S—, —NR^{T2′}—, —C(═O)—, —S(═O)—, —SO₂—, —C(═O)O—, —C(═O)NR^{T2′}—, —OC(═O)—, —NR^{T2′}C(═O)—, —OC(═O)O—, —OC(═O)NR^{T2′}—, —NR^{T2′}C(═O)O—, —NR^{T2′}C(═O)NR^{T3′}—, —C(═S)—, —C(═S)S—, —SC(═S)—, —SC(═S)S—, —C(═NR^{T2′})—, —C(═NR^{T2′})O—, —C(═NR^{T2′})NR^{T3′}—, —OC(═NR^{T2′})—, —NR^{T2′}C(═NR^{T3′})—, —NR^{T2′}SO₂—, —C(═NR^{T2′})NR^{T3′}—, —OC(═NR^{T2′})—, —NR^{T2′}C(═NR^{T3′})—, —NR^{T2′}SO₂—, —NR^{T2′}SO₂NR^{T3′}—, —NR^{T2′}C(═S)—, —SC(═S)NR^{T2′}—, —NR^{T2′}C(═S)S—, —NR^{T2′}C(═S)NR^{T3′}—, —SC(═NR^{T2′})—, —C(═S)NR^{T2′}—, —OC(═S)NR^{T2′}—, —NR^{T2′}C(═S)O—, —SC(═O)NR^{T2′}—, —NR^{T2′}C(═O)S—, —C(═O)S—, —SC(═O)—, —SC(═O)S—, —C(═S)O—, —OC(═S)—, —OC(═S)O—, —SO₂NR^{T2′}—, —BR^{T2′}—, or —PR^{T2′}—; where each occurrence of R^{T1′}, R^{T2′}, and R^{T3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some instances, “substituted” also refers to one or more substitutions of one or more of the carbon atoms in a carbon chain (e.g., alkyl, alkenyl, alkynyl, and aryl groups) by a heteroatom, such as, but not limited to, nitrogen, oxygen, and sulfur.

It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### II. Functionalized Compounds

tRNA acylated with aromatic and polyketo functional molecules and methods of making them are provided. Functionalized polypeptides incorporating one or more functional molecules at the N-terminus, C-terminus, one or more internal residues (not the N-terminus or C-terminus), or a combination thereof are also provided.

Conventionally, the covalent attachment of an amino acid to a tRNA's 3′ end is catalyzed by enzymes called aminoacyl tRNA synthetases. During protein synthesis, tRNAs with attached amino acids are delivered to the ribosome by elongation factors, which aid in association of the tRNA with the ribosome, synthesis of the new polypeptide, and translocation of the ribosome along the mRNA. If the tRNA's anticodon matches the mRNA, another tRNA already bound to the ribosome transfers the growing polypeptide chain from its 3′ end to the amino acid attached to the 3′ end of the newly delivered tRNA, a reaction catalyzed by the ribosome.

The experiments below show that a tRNA, such as an initiator tRNA, charged with functional molecules including benzoic acid, malonatic acid, and derivatives thereof can also participate in translation.

For example, functionalized initiator tRNA can bind directly to the P site of ribosomes and transfer the functional molecule from the tRNA's 3′ end to the functional molecule (which may be an amino acid, peptide, or non-peptide polymer) attached to the 3′ end of the newly delivered tRNA in the A site. Translation can then proceed with additional standard or non-standard amino acids or other functional molecules, or a combination thereof added to the growing chain. In this way, a functional molecule forms the N-terminus of a new hybrid polypeptide or other sequence-defined polymer.

The experiments below also show that a tRNA, preferably an elongator tRNA, charged with a functional molecule can also participate in translation. Functionalized elongator tRNA can bind to the A site of ribosomes and the functional molecule attached to the 3′ end of the functionalized tRNA can receive the functional molecule (which may be an amino acid, peptide, or non-peptide polymer) attached to the 3′ end of preceding tRNA resident in the P site. Translation can terminate or proceed with additional standard or non-standard amino acids or other functional molecules, or a combination thereof, added to the growing chain. The functional molecule forms the C-terminus and/or internal residue(s) of the new hybrid polypeptide or other sequence defined polymer.

A. Sources and Selection of Uncharged tRNA

A transfer RNA is an adaptor molecule composed of RNA, typically 76 to 90 nucleotides in length, that serves as the physical link between the mRNA and the amino acid sequence of proteins. tRNA does this by carrying an amino acid to the ribosome as directed by a 3-nucleotide codon in an mRNA. It has been discovered that instead of a cognate amino acid, tRNA, including wildtype tRNA, can also be charged with functional molecules such as chemical monomers and chemical-amino acid hybrids, which can be incorporated at the N-terminus or C-terminus of a polypeptide, or internally, during translation by wildtype ribosomes. The functional molecules do not consist of a canonical amino acid, and can also be distinct from non-standard amino acids. Typically, the molecule consists or comprises a benzoic acid or benzoic acid derivative, or a malonic acid or malonic acid derivative.

Naturally occurring and non-naturally occurring (e.g., genetically engineered) tRNA and tRNA-like molecules can be used.

In some embodiments, the tRNA is from, for example, a prokaryote or a eukaryote, or is a variant thereof with a substituted anticodon.

Sequences for such tRNAs are well known in the art. For example, C. elegans has 620 genes encoding for tRNA, Saccharomyces cerevisiae has 275 tRNA genes in its genome, and humans have at least 497 nuclear genes encoding cytoplasmic tRNA molecules and 22 mitochondrial tRNA genes encoding mitochondrial tRNAs. E. coli typically has at least 79 tRNA and often more depending on the strain.

In some embodiments, the engineered tRNA has the same sequence as a naturally occurring counterpart except for the anticodon sequence, which is substituted for an alternative anticodon. The alternative anticodon can be one that recognizes an amino acid codon or a stop codon.

Other non-naturally occurring tRNA suitable for use in the disclosed methods are also known in the art, see, for example, Dumas, et al., Chem. Sci., 6:50-69 (2015), Liu and Schultz, Annu. Rev. Biochem., 79:413-44 (2010), Davis and Chin, Nat. Rev. Mol. Cell Biol., 13:168-82 (2012), WO 2015/120287, U.S. Pat. Nos. 9,464,288, 10,240,158, and 10,023,893, and U.S. Published Application No. 2018/0105854.

In some embodiments, the tRNA is one described in Tharp, et al., “Initiation of Protein Synthesis with Non-Canonical Amino Acids In Vivo,” Angew Chem Int Ed Engl., 2020 Feb. 17; 59(8):3122-3126. doi: 10.1002/anie.201914671. Epub 2020 Jan. 21, Accepted manuscript online: Dec. 11, 2019, or the Supporting Information (anie_201914671_sm_miscellaneous_information.pdf), published therewith, each of which is specifically incorporated by reference herein in its entirety, and including all tRNAs, aminoacyl-tRNA synthetase (aaRS or ARS), and other compositions, methods, and materials discussed therein. Also provided are tRNAs and AARS with at least 70%, 75%, 80%, 85%, 90%, 95%, or 100% sequence identity to a tRNA or AARS described in Tharp, et al., supra.

The tRNA can be an initiator tRNA, an elegator tRNA, or suppressor tRNA. The initiator tRNA performs functions different from those of any other tRNA. It is the only tRNA that binds directly to the P site of the ribosome during the translational cycle; it is also one of the only tRNAs that must avoid binding to elongation factor Tu (EF-Tu in bacteria; eEF1A in eukaryotes). In addition, the initiator tRNA is typically distinguishable from the other methionine-bearing tRNA present in the cytoplasm, the elongator methionyl tRNA that contributes methionine residues during peptide chain elongation.

Charged, elongator tRNAs are delivered to the ribosomal A site, not the P site. In eukaryotes, elongator tRNAs are delivered to the A site in complex with GTP-bound eukaryotic elongation factor 1A (eEF1A). In bacterial elongation, the homologous EF-Tu serves this role.

Thus, in more particular embodiments, an initiator tRNA can be selected as the tRNA for functionalization when the functional molecule is desired at the N-terminus. In other embodiments, an elongator tRNA is selected as the tRNA for functionalization when the functional is desired at the C-terminus or internally. Preferably, the functionalized elongator tRNA can still be delivered to A site by an elongation (Tu) factor.

B. Functional Molecules

Functional molecules are provided. The functional molecules typically include a benzoic acid or benzoic acid derivative, or a malonic acid or malonic acid derivative. The functional molecules may or may not include other moieties such as one or more standard or non-standard amino acids.

The functional molecules can be acylated to the 3′ end of a tRNA (e.g, at the 2′ or 3′ position of the terminal nucleotide's ribose or at the 3′ amine) to form a functionalized tRNA. The functional molecules can also form part of the growing polypeptide during translation. Thus, formulas for functionalized compounds including both functionalized tRNA and the corresponding functionalized (hybrid) polypeptides or other sequence defined polymer incorporating the functionalized molecule are provided.

Exemplary functionalized tRNA and polypeptides (collective referred to as functinalized compounds) are provided below.

The tRNA formulae illustrate a functional molecule (e.g., benzoic acid or a benzoic acid derivative or malonic acid or a malonic acid derivative) linked to the 3′ adenosine (e.g, at the 2′ or 3′ position of the terminal nucleotide's ribose or at the 3′ amine of the adenine nucleobase) of a tRNA, or linked to an amino acid, wherein the amino acid is linked (e.g., acylated) to the tRNA. The remaining portion of the tRNA that is 5′ to the terminal 3′ nucleotide (e.g, illustrated with adenosine in the formulae) is denoted by the label “tRNA” in the formulae. Thus, the “tRNA” label of the formulae in combination with the terminal 3′ nucleotide (e.g, illustrated with adenosine in the formulae) can be the remaining portion of the parent (i.e., unacylated) tRNA prior to functionalization. The parent tRNA of the formulea can be a natural or engineered tRNA or tRNA-like molecule. It will be appreciated that the 3′ nucleobase need not be adenine. Thus, although not illustrated below, each formulae wherein the 3′ terminal adenine is replaced with a cytosine, guanine, or uracil is also expressly provided. Thus, the 3′ end (i.e., 3′ nucleotide) of the parent tRNA can be adenosine, guanosine, uridine, or cytidine. In cases where the functional molecule is linked to an amino group of the 3′ nucleobase, the amino group is typically a primary amino group (e.g., as found in adenine, cytosine, and guanine).

Exemplary polypeptides with a benzoic acid or a benzoic acid derivative or malonic acid or a malonic acid derivative are provided below. The formulae illustrate a single functional molecule linked only to the N-terminus or C-terminus of a polypeptide. However, polypeptides and other sequence defined polymers having two or more of the same or different functional molecules at the N-terminus, C-terminus, at one or more internal positions or any combination thereof are also provided. The polypeptides and sequence defined polymers can include one or more standard amino acids, non-standard amino acids, functional molecules, or combinations thereof.

1. Benzoic Acid and Derivatives Thereof

In particular, the disclosed functionalized compounds have a structure of Formula I:

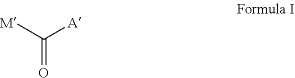

where M′ is a tRNA or a polypeptide; and

where A′ is an unsubstituted aryl group, a substituted aryl group, an unsubstituted heteroaryl group, or a substituted heteroaryl group.

a. Exemplary Functionalized tRNA

In some forms, M′ is a tRNA and the functionalized tRNA has a structure of Formula II, Formula II′, or Formula II″:

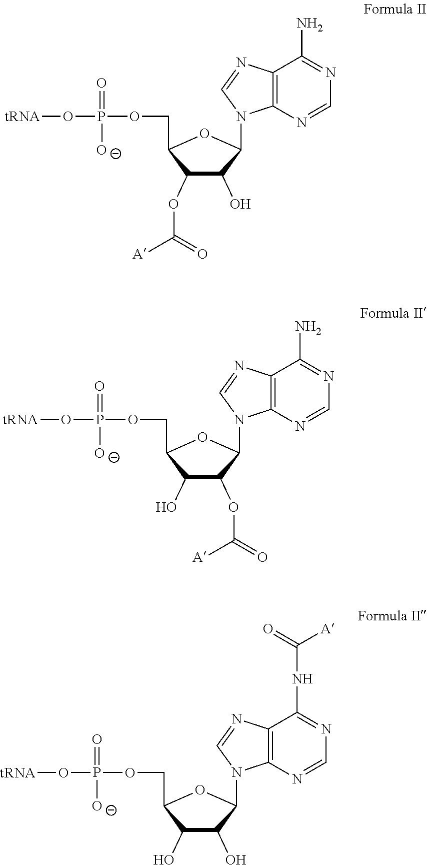

where A′ is as defined above.

In some forms, A′ is an unsubstituted aryl group or a substituted aryl group. In some forms, A′ is a substituted aryl group.

In some forms, the functionalized tRNAs have a structure of Formula III, III′, or III″:

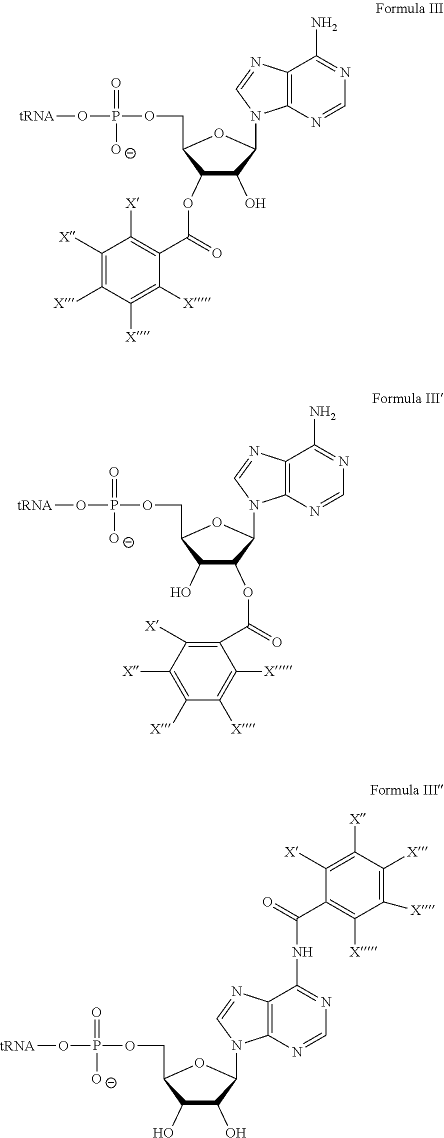

where X′, X″, X′″, X″″, and X are independently a hydrogen atom, a deuterium atom, a tritium atom, or a halogen atom selected from fluorine, chlorine, bromine, and iodine.

In some forms, X′ is fluorine.

In some forms, the functionalized tRNAs have a structure of Formula IV, Formula IV′, or Formula IV″:

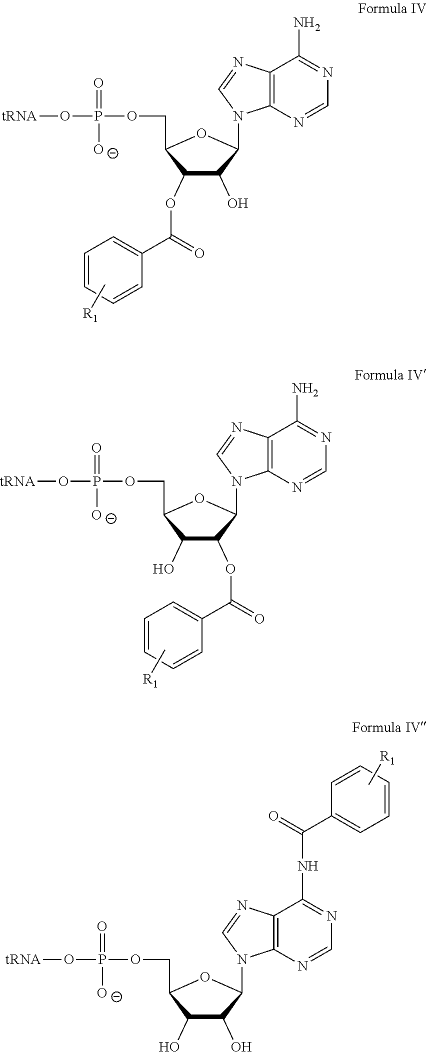

where R₁ is a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

In some forms, R₁ is not a hydrogen bond donor.

In some forms, R₁ is a primary amine. In some forms, R₁ is an ortho-primary amine. In some forms, R₁ is a halogen atom. In some forms, R₁ is chloride. In some forms, R₁ is para-chloride. In some forms, R₁ is a nitro group. In some forms, R₁ is an azide group. In some forms, R₁ is a methyl azide group. In some forms, R₁ is an ether group. In some forms, R₁ is a methoxy group. In some forms, R₁ is an alkyl group. In some forms, R₁ is a methyl group. In some forms, R₁ includes one or more acidic protons. In some forms, R₁ includes one or more ammonia cations.

In some forms, A′ is an unsubstituted heteroaryl group or a substituted hereoaryl group. In some forms, A′ is a substituted heteroaryl group.

In some forms, the functionalized tRNAs have a structure of Formula V, Formula V′, or Formula V″:

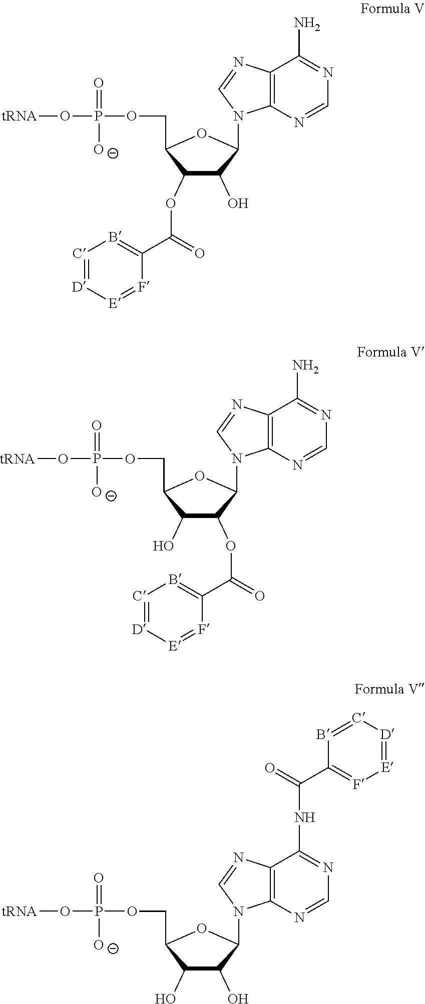

where B′, C′, D′, E′, and F′ are independently C—R₁ or a nitrogen atom;
where R₁ is as defined above; and
where at least one of B′, C′, D′, E′, and F′ is a nitrogen atom.

In some forms, the functionalized compounds are functionalized tRNAs having a structure of Formula XII:

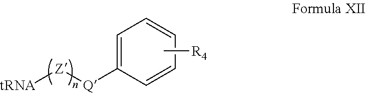

where each Z′ is an amino acid;

where n is an integer between 1 and 4 inclusive, preferably 1;

where Q′ is an amide group or an ester group; and

where R₄ includes an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

In some forms, R₄ includes a primary amine.

b. Exemplary Functionalized Polypeptides

In some forms, M′ is a polypeptide and the functionalized polypeptide has a structure of Formula VI:

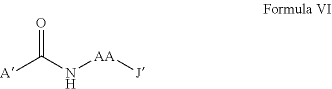

where A′ is as defined above;

where NH-AA is an amino acid linked to the functional molecule through a peptide bond; and

where J′ is one or more amino acids.

In some forms, A′ is an unsubstituted aryl group or a substituted aryl group. In some forms, A′ is a substituted aryl group.

In some forms, the functionalized polypeptides have a structure of Formula VII:

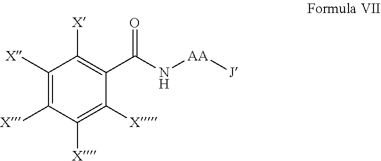

where NH-AA and J′ are as defined above; and

where X′, X″, X′″, X″″, and X′″″ are independently a hydrogen atom, a deuterium atom, a tritium atom, or a halogen atom selected from fluorine, chlorine, bromine, and iodine.

In some forms, X′ is fluorine.

In some forms, the functionalized polypeptides have a structure of Formula VIII:

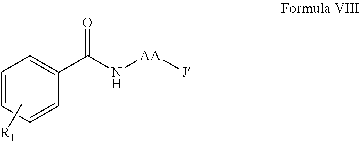

where NH-AA and J′ are as defined above; and

where R₁ is a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

In some forms, R₁ is not a hydrogen bond donor.

In some forms, R₁ is a primary amine. In some forms, R₁ is an ortho-primary amine. In some forms, R₁ is a halogen atom. In some forms, R₁ is chloride. In some forms, R₁ is para-chloride. In some forms, R₁ is a nitro group. In some forms, R₁ is an azide group. In some forms, R₁ is a methyl azide group. In some forms, R₁ is an ether group. In some forms, R₁ is a methoxy group. In some forms, R₁ is an alkyl group. In some forms, R₁ is a methyl group. In some forms, R₁ includes one or more acidic protons. In some forms, R₁ includes one or more ammonia cations.

In some forms, A′ is an unsubstituted heteroaryl group or a substituted hereoaryl group. In some forms, A′ is a substituted heteroaryl group.

In some forms, the functionalized polypeptides have a structure of Formula IX:

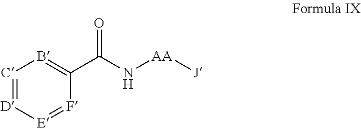

where NH-AA and J′ are as defined above;
where B′, C′, D′, E′, and F′ are independently C—R₁ or a nitrogen atom;
where R₁ is as defined above; and
where at least one of B′, C′, D′, E′, and F′ is a nitrogen atom.

In some forms, the functionalized compounds are functionalized polypeptides having a structure of Formula XII′:

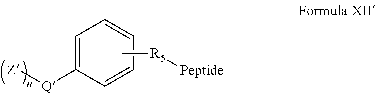

where Z′, n, and Q′ are as defined above; and

where R₅ includes a secondary amino group optionally containing a substituent at the amino nitrogen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group.

2. Malonic acid and Malonic acid Derivatives

a. Exemplary tRNA

In some forms, the functionalized tRNAs have a structure of Formula XIV, XIV′, or XIV″:

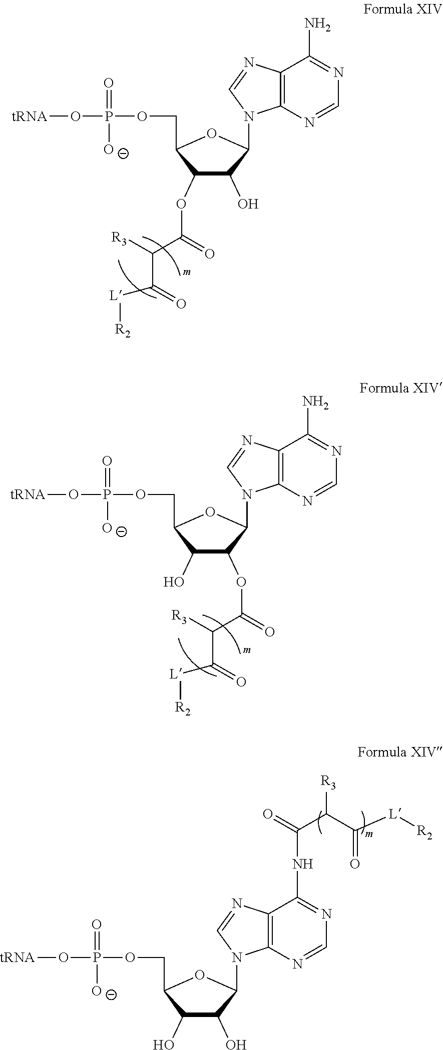

where L′ is an oxygen atom, a nitrogen atom, or a sulfur atom;

where m is an integer between 1 and 10 inclusive; and

where R₂ and each R₃ are independently:

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

In some forms, m is 1. In some forms, R₃ is a hydrogen atom. In some forms, R₃ is a hydrogen atom and m is 1. In some forms, R₂ is a substituted aryl group. In some forms, L′ is an oxygen atom. In some forms, L′ is a sulfur atom.

In some forms, the functionalized compounds are functionalized tRNAs having a structure of Formula XIII:

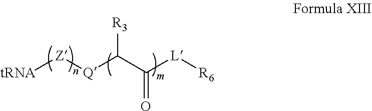

where Z′, Q′, L′, and R₃ are as defined above; and

R₆ is hydrogen or includes an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

b. Exemplary Functionalized Polypeptides

In some forms, the functionalized polypeptide have a structure of Formula XI:

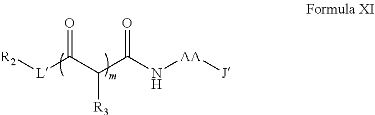

where NH-AA, J′, L′, R₂, R₃, and m are as defined above.

In some forms, the functionalized compounds are functionalized polypeptides having a structure of Formula XIII′:

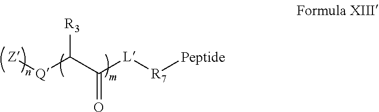

where Z′, Q′, L′, and R₃ are as defined above; and

where R₇ is absent or includes a secondary amino group optionally containing a substituent at the amino nitrogen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group.

C. Methods of Attaching Functional Molecules to tRNA

Functionalized tRNA can be prepared by an enzymatic or chemical reaction.

1. Enzymatic Means

In some embodiments, the acylation of a functional molecule to an uncharged tRNA is carried by enzymatic means. In some embodiments, the enzyme is a flexizyme.

Flexizymes are versatile ribozymes, and have been shown to be capable of synthesizing aminoacyl-tRNA using pre-activated amino acid substrates (Saito, et al., EMBO J. 20:1797-1806 (2001); Saito and Suga, J. Am. Chem. Soc., 123:7178-7179 (2001); Murakami, et al., Chem. Biol., 10:655-662 (2003); Murakami, et al., Nat. Methods; 3:357-359 (2006); Xiao, et al., Nature, 454:358-361 (2008); Niwa, Bioorg. Med. Chem. Lett., 19:3892-3894 (2009); Goto, et al., Nature Protoc.; 6:779-790 (2011), Katoh and Suga, Nucleic Acids Research, 47(9):e54 (2019)). Compared with ARSs, acylation occurs specifically at the 3′-position of the 3′-terminal adenosine, while class I enzymes acylate at the 2′ and class II at the 3′-position.

Flexizymes were evolved from pools of random RNA sequences through in vitro selection. Several flexizymes are available, including eFx and dFx. Reported substrates of eFx are amino acid cyanomethyl esters (CMEs) or 4-chlorobenzyl thioesters (CBTs), while dFx utilizes amino acid dinitrobenzyl esters (DBEs). Since eFx and dFx recognize only the conserved 3′-terminal CCA region of tRNAs, any type of tRNA or shorter RNAs with CCA ends can be used as substrates. eFx and dFx have been shown to charge proteinogenic and nonproteinogenic aminoacyl-donors onto tRNAs, which can be used to generate peptides with or without nonproteinogenic amino acids (Katoh and Suga, Nucleic Acids Research, 47(9):e54 (2019)).

The experiments below show that flexizymes can charge tRNA with non-amino acid functional molecules. In some embodiments, the flexiyme is eFx or dFx.

dFx
(SEQ ID NO: 1)
GGAUCGAAAGAUUUCCGCAUCCCCGAAAGGGUACAUGGCGUUAGGU.
eFx
(SEQ ID NO: 2)
GGAUCGAAAGAUUUCCGCGGCCCCGAAAGGGGAUUAGCGUUAGGU.
aFx
(SEQ ID NO: 3)
GGAUCGAAAGAUUUCCGCACCCCCGAAAGGGGUAAGUGGCGUUAGGU.

An acylation reaction can include, for example, mixing flexizyme uncharged tRNA, the desired functional molecule precursor, and magnesium chloride (MgCl) in a suitable buffer (e.g., HEPES or Bicine). In some embodiments, the flexizyme and uncharged tRNA are first mixed, heated, and cooled prior to the addition of the functional molecule precursor and MgCl. A specific exemplary protocol is provided in the experiments below.

Additionally, or alternatively, in some embodiments, the tRNA is charged by a protein enzyme such as an orthogonal amino acyl tRNA synthetase.

Successful acylation can be confirmed using any suitable means including but not limited to gel shift assays, LC-MS, etc.

2. Chemical Means

In some embodiments, the acylation of a functional molecule to an uncharged tRNA is carried by a non-enzymatic chemical reaction.

In particularly embodiments, isatoic anhydride is used to prepare anthraninoyl-tRNA. Generally, uncharged tRNA are incubated in a base solution (e.g., incubated with 2-5 mM NaOH in 90% acetonitrile) with an effective amount of isatoic anhydride under suitable conditions (e.g., time and temperature) to acylate the tRNA. The sample can be diluted in water, flash frozen, lyophilized, and resuspended in a suitable buffer for use. See, also (Young et al., ACS Chem. Biol., 13, 854-870 (2018)), and a specific exemplary protocol is provided in the experiments below.

D. Nucleic Acids and Polypeptides of Interest

1. Nucleic Acids Encoding a Hybrid Polypeptide of Interest

The functionalized tRNA can be used in combination with an mRNA to manufacture hybrid polypeptides incorporating the functional molecule at the N-terminus, the C-terminus, internal sites, or a combination thereof. In some embodiments, the mRNA is added to the translation system, which can be free from DNA encoding the mRNA. In some embodiments, DNA encoding the mRNA is transcribed by the system. Thus, although typically discussed below as mRNA, the corresponding DNA sequences, optionally further include expression control sequences, are also expressly provided herein and can utilized in the disclosed translation systems as part of transcription/translation reaction.

The mRNA, which encodes a hybrid polypeptide of interest, includes one or more codons that is recognized by the anticodon of the functionalized tRNA, referred to herein as a “functionalized tRNA recognition codon,” such that the functionalized tRNA, when used in combination with other translation factors, facilitates the attachment of the functional molecule to the growing polypeptide chain during translation.

A functionalized tRNA recognition codon can be at the beginning (i.e., first 5′ codon), the end (i.e., last 3′ codon), at one or more internal codons, or any combination thereof of the coding region of the mRNA to facilitate functionalization of the N-terminus, C-terminus, one or more internal sites/residues or combination thereof, respectively, of the hybrid polypeptide. Thus, in some embodiments, the mRNA encodes 1, 2, 3, 4, 5, 6, 7, 8, 9, 10, or more functionalized tRNA recognition codons. Any one or more of the functionalized tRNA recognition codons can be the same or different, thus incorporating the same or different functional molecules into the translated molecule, respectively. In some embodiments, there is at least one, two, three, four, five or more codons (e.g., encoding a naturally or non-naturally occurring amino acid) between the two functionalized tRNA recognition codons. In some embodiments, two or more adjacent codons encode functional molecules. In some embodiments, codons encoding functional molecules are not adjacent.

In some embodiments, the functionalized tRNA recognition codon is a stop codon. When the tRNA recognition codon is a stop codon, such as UGA, the mRNA will contain at least one UGA codon where a functional molecule will be added to the growing polypeptide chain during translation. Although illustrated using a stop codon (and a suppressor tRNA), the functionalized tRNA recognition codon can be any codon sequence provided it is recognized by the anticodon of the functionalized tRNA during translation. The tRNA also need not be a suppressor tRNA.

In some embodiments, the mRNA can include or consist of replacing of the AUG start codon with GUG or UUG and optionally a UAAUU inserted in front of it. Replacing AUG with GUG or UUG can reduce the expression of the encoded protein.

Various types of mutagenesis can be used to modify the sequence of a nucleic acid encoding the mRNA of interest to generate functionalized tRNA recognition codons. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, and mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis and double-strand break repair.

In some embodiments, the coding sequence, excluding the tRNA recognition site as discussed above, is further altered for optimal expression (also referred to herein as “codon optimized”) in an expression system of interest. Methods for modifying coding sequences to achieve optimal expression are known in the art.

2. Hybrid Polypeptides and Other Polymers

The sequence of the mRNA and DNA is typically determined by first determining the desired hybrid polypeptide sequence, including the sequence of the desired polypeptide and the location of the desired functional molecule.

The polypeptide of interest can have the sequence of a known naturally occurring or engineered or recombinant polypeptide or protein, or a new previously unknown sequence, for example a random sequence of amino acids.

In some embodiments, the sequence of the hybrid polypeptide is designed to include or form specific desired secondary, tertiary, or quaternary structures, or a combination thereof.

In some embodiments, the hybrid polypeptide is designed to form a cyclic polypeptide.

The polypeptide can be any desired length. For example, in some embodiments, the hybrid polypeptide includes between about 1 and 1,000 amino acids inclusive, or any specific integer of amino acids there between, or any specific range of two integers there between.

As discussed herein, the functional molecule can incorporated at the N-terminus, C-terminus, one or more internal residues, or any combination thereof, of the polypeptide.

In some embodiments, the functionalized tRNA includes zero, one, two, three, four, or more amino acids, and thus the functional molecule can include zero, one, two, three, four, or more amino acids. Thus, the polypeptide may begin and terminate with one, two, three, four, or more amino acids, which are incorporated as part of the functional molecule during elongation. In some embodiments, the functionalized tRNA does not include any amino acid(s). In some embodiments, the polypeptide begins with and/or ends with a non-amino acid functional molecule.

In some embodiments, the translated molecule includes 1, 2, 3, 4, 5, 6, 7, 8, 9, 10, or more functional molecule units. In some embodiments, N-terminus of the encoded polypeptide and/or the C-terminus and/or one or more internal sites of the encoded polypeptide is a functional molecule. In some embodiments, two or more adjacent residues are functional molecules. In some embodiments, functional molecules are not adjacent.

### III. Methods for Manufacturing Hybrid Polypeptides and Other Sequence Defined Polymers

Generally, canonical amino acids are charged onto their respective tRNA by their cognate aminoacyl-tRNA synthetase. The aminoacyl-tRNA is then delivered by EF-Tu to the ribosome. However, the experiments below illustrate that through both chemical and flexizyme reactions, naturally-occurring tRNAs can be charged with functional molecules and incorporated into the N-terminal and/or C-terminal position of a growing polypeptide during translation. Thus, the disclosed compositions and methods can be used to prepare hybrid polypeptides and other sequence defined polymers including a combination of the functional molecule and standard or non-standard amino acids.

As discussed in more detail below, hybrid polypeptides can be prepared using in vitro transcription/translation or in vivo expression systems. The system can be of prokaryotic, eukaryotic, or archaeal origin or combinations thereof. For example, the system can be a hybrid system including translation factors from two or more of prokaryotic, eukaryotic, and archaeal origin.

It is understood that if the functionalized tRNA recognition codon of the mRNA of interest is one of the three mRNA stop codons (UAG, UAA, or UGA) translation of some of the mRNA of interest will terminate at the functionalized tRNA recognition codon causing failed initiation or a polypeptide that does not include the functional molecule. In some embodiments, the hybrid polypeptide is expressed in a system that has been modified or mutated to reduce or eliminate expression of one or more translation release factors. A release factor is a protein that allows for the termination of translation by recognizing the termination codon or stop codon in an mRNA sequence. Prokaryotic release factors include RF1, RF2 and RF3; and eukaryotic release factors include eRF1 and eRF3. Deletion of one or more release factors may result in “read-through” of the intended stop codon.

The polypeptide of interest can be purified from non-functionalized proteins and other contaminants using standard methods of protein purification as discussed in more detail below.

A. In vitro Transcription/Translation

The Examples below illustrate that wildtype tRNA can be functionalized by chemical and enzymatic methods, and the functionalized tRNA can transfer the functional molecule the N-terminus or C-terminus of the grouping polypeptide during translation utilizing wildtype translation factors including wildtype ribosomes.

In vitro translation typically includes provision of the mRNA encoding the polypeptide of interest. The mRNA can be provided directly, or can be provided indirectly in the form of DNA encoding polypeptide of interest which if first transcribed in vitro to produce the mRNA, which is then translated.

In vitro protein synthesis does not depend on having a polyadenylated mRNA, but if having a poly(A) tail is important for some other purpose a vector may be used that has a stretch of, for example, 100 A residues incorporated into the polylinker region. That way, the poly(A) tail is “built in” by the synthetic method.

Eukaryotic ribosomes read RNAs more efficiently if they have a 5′ methyl guanosine cap. RNA caps can be incorporated by initiation of transcription using a capped base analogue, or adding a cap in a separate in vitro reaction post-transcriptionally.

The use of in vitro translation systems can have advantages over in vivo gene expression when the over-expressed product is toxic to the host cell, when the product is insoluble or forms inclusion bodies, or when the protein undergoes rapid proteolytic degradation by intracellular proteases.

Cell-free translation systems typically include contain all the macromolecular components (70S or 80S ribosomes, tRNAs, aminoacyl-tRNA synthetases, initiation, elongation and termination factors, etc.) required for translation of exogenous RNA. To ensure efficient translation, each extract is typically supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors (Mg2+, K+, etc.).

Exemplary suitable in vitro transcription/translation systems include, but are not limited to, the rabbit reticulocyte system, the E. coli-based systems (e.g., S-30 transcription-translation system), and the wheat germ based translational system.

In vitro protein synthesis can include translation of purified RNA, as well as “linked” and “coupled” transcription:translation. In vitro translation systems can be eukaryotic or prokaryotic cell-free systems. Combined transcription/translation systems are available, in which both phage RNA polymerases (such as T7 or SP6) and translation components are present. One example of a kit is the TNT® system from Promega Corporation. The experiments below utilize the commercial available transcription/translation system PUREXPRESS® by New England Biolabs with some modifications.

Generally, to generate hybrid polypeptides, translation components are provided in combination with a template DNA or mRNA for the hybrid polypeptide. The functionalized tRNA can be provided pre-charged with the desired functional molecule. In some embodiments, one or more translation components are omitted to permit or enhance incorporation of the functionalized group.

In some embodiments, the uncharged tRNA corresponding to the provided functionalized tRNA, and/or its associated AARS, and/or the amino acid with which it is typically charged is omitted from the system. For example, when a methionine tRNA is charged with a functional molecule to form of a functionalized acyl-tRNA^{met}, one or more of uncharged tRNA^{met}, AARS^{met}, or methionine may be omitted from the reaction, particularly where the hybrid polypeptide does not include a methionine. In some embodiments, only the naturally occurring uncharged tRNA with the same anticodon is omitted from the reaction. For example, if the functionalized tRNA is a tRNA^{val}_{UAC}, only uncharged tRNA^{Val}_{UAC} is omitted from the reaction. Thus, the codon GUA can be used to encode the functional molecule, while other valine encoding codons (e.g., GUU, GUC, GUG) can be utilized to incorporate valine using the corresponding tRNA^{val}.

In some embodiments, wherein the functionalized tRNA features a stop anticodon, one or more release factors may be omitted from the reaction.

B. In Vivo Methods Transcription/Translation

Host cells can be transformed, transduced or transfected with the vectors or genetically engineering to express nucleic acid sequences encoding the additional components necessary to carry out hybrid polypeptide expression in vivo. For example, in some embodiments, a DNA construct encoding the hybrid polypeptide and one or more flexizyme or a protein enzyme such as an orthogonal amino acyl tRNA synthetase are expressed by host cells. The functional molecule, or a precursor thereof, that is functionalized to the target tRNA can be added as a supplement (e.g., to the host cells' media), or expressed by the cells (e.g., through an appropriate biosynthetic pathway). In some embodiments, the cell also expresses one or more non-naturally occurring tRNA, for example a tRNA having a stop anticodon, that can hybridize with a target stop codon encoded by hybrid polypeptide mRNA

1. Forms of Expression

In vivo methods can include extrachromosomal expression, genomic expression, or a combination thereof of translation components.

a. Extrachromosomal Expression

Any one or more of the naturally-occurring and/or engineered translation components can be expressed extrachomosomally, for example, from a vector or vectors. The vector can be, for example, in the form of a plasmid, a bacterium, a virus, a naked polynucleotide, or a conjugated polynucleotide. The vectors are introduced into cells and/or microorganisms by standard methods including electroporation, infection by viral vectors, high velocity ballistic penetration by small particles with the nucleic acid either within the matrix of small beads or particles, or on the surface.

Nucleic acids in vectors can be operably linked to one or more expression control sequences. Operably linked means the disclosed sequences are incorporated into a genetic construct so that expression control sequences effectively control expression of a sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). Some promoters are “constitutive,” and direct transcription in the absence of regulatory influences. Some promoters are “tissue specific,” and initiate transcription exclusively or selectively in one or a few tissue types. Some promoters are “inducible,” and achieve gene transcription under the influence of an inducer. Induction can occur, e.g., as the result of a physiologic response, a response to outside signals, or as the result of artificial manipulation. Some promoters respond to the presence of tetracycline; “rtTA” is a reverse tetracycline controlled transactivator. Such promoters are well known to those of skill in the art.

To bring a coding sequence under the control of a promoter, it is advantageous to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Likewise, although tRNA sequences do not encode a protein, control sequence can be operably linked to a sequence encoding a tRNA, to control expression of the tRNA in a host cell. Methods of recombinant expression of tRNA from vectors is known in the art, see for example, Ponchon and Dardel, Nature Methods, 4(7):571-6 (2007); Masson and Miller, J. H., Gene, 47:179-183 (1986); Meinnel, et al., Nucleic Acids Res., 16:8095-6 (1988); Tisnd, et al., RNA, 6:1403-1412 (2000).

Methods of expressing recombinant proteins in various recombinant expression systems including bacteria, yeast, insect, and mammalian cells are known in the art, see for example Current Protocols in Protein Science (Print ISSN: 1934-3655 Online ISSN: 1934-3663, January 2012). Plasmids can be high copy number or low copy number plasmids. In some embodiments, a low copy number plasmid generates between about 1 and about 20 copies per cell (e.g., approximately 5-8 copies per cell). In some embodiments, a high copy number plasmid generates at least about 100, 500, 1,000 or more copies per cell (e.g., approximately 100 to about 1,000 copies per cell).

Kits are commercially available for the purification of plasmids from bacteria, (see, e.g., GFX™ Micro Plasmid Prep Kit from GE Healthcare; Strataprep® Plasmid Miniprep Kit and StrataPrep® EF Plasmid Midiprep Kit from Stratagene; GenElute™ HP Plasmid Midiprep and Maxiprep Kits from Sigma-Aldrich, and, Qiagen plasmid prep kits and QIAfilter™ kits from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems.

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express tRNA and mRNA for producing hybrid proteins or polypeptides. Viral based expression systems are well known in the art and include, but are not limited to, baculoviral, SV40, retroviral, or vaccinia based viral vectors.

Mammalian cell lines that stably express tRNA and mRNA or interest and other components can be produced using expression vectors with appropriate control elements and a selectable marker. For example, the eukaryotic expression vectors pCR3.1 and p91023(B) are suitable for expression of recombinant proteins in, for example, Chinese hamster ovary (CHO) cells, COS-1 cells, human embryonic kidney 293 cells, NIH3T3 cells, BHK21 cells, MDCK cells, and human vascular endothelial cells (HUVEC). Additional suitable expression systems include the GS Gene Expression System™ available through Lonza Group Ltd.

U6 and H1 are exemplary promoters that can be used for expressing bacterial tRNA in mammalian cells.

Following introduction of an expression vector by electroporation, lipofection, calcium phosphate, or calcium chloride co-precipitation, DEAE dextran, or other suitable transfection method, stable cell lines can be selected (e.g., by metabolic selection, or antibiotic resistance to G418, kanamycin, or hygromycin or by metabolic selection using the Glutamine Synthetase-NS0 system). The transfected cells can be cultured such that the polypeptide of interest is expressed, and the polypeptide can be recovered from, for example, the cell culture supernatant or from lysed cells.

b. Expression by Genomic Integration

Any one or more of the naturally occurring and/or engineered translation components can be expressed from one or more genomic copies. Methods of engineering microorganisms or cell lines to incorporate a nucleic acid sequence into its genome are known in the art. Nucleic acids that are delivered to cells which are to be integrated into the host cell genome can contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome. In some embodiments, the systems are designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome.

For example, cloning vectors expressing a transposase and containing a nucleic acid sequence of interest between inverted repeats transposable by the transposase can be used to clone the stably insert the gene of interest into a bacterial genome. Stably insertion can be obtained using elements derived from transposons including, but not limited to Tn7. Additional methods for inserting heterologous nucleic acid sequences in E. coli and other gram-negative bacteria include use of specialized lambda phage cloning vectors that can exist stably in the lysogenic state. Integrative plasmids can be used to incorporate nucleic acid sequences into yeast chromosomes. Methods of incorporating nucleic acid sequence into the genomes of mammalian lines are also well known in the art using, for example, engineered retroviruses such lentiviruses.

2. Host Cells

Host cell including the nucleic acids disclosed herein are also provided. Prokaryotes useful as host cells include, but are not limited to, gram negative or gram positive organisms such as E. coli or Bacilli. In a prokaryotic host cell, a polypeptide may include an N-terminal methionine residue to facilitate expression of the recombinant polypeptide in the prokaryotic host cell. The N-terminal Met may be cleaved from the expressed recombinant polypeptide. Promoter sequences commonly used for recombinant prokaryotic host cell expression vectors include lactamase and the lactose promoter system.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Commercially available vectors include, for example, T7 expression vectors from Invitrogen, pET vectors from Novagen and pALTER® vectors and PinPoint® vectors from Promega Corporation.

In some embodiments, the host cells are E. coli. The E. coli strain can be a selA, selB, selC, deletion strain, or combinations thereof. For example, the E. coli can be a selA, selB, and selC deletion strain, or a selB and selC deletion strain. Examples of suitable E. coli strains include, but are not limited to, MH5 and ME6.

Yeasts useful as host cells include, but are not limited to, those from the genus Saccharomyces, Pichia, K. Actinomycetes and Kluyveromyces. Yeast vectors will often contain an origin of replication sequence, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Fleer et al., Gene, 107:285-195 (1991), in Li, et al., Lett Appl Microbiol. 40(5):347-52 (2005), Jansen, et al., Gene 344:43-51 (2005) and Daly and Hearn, J. Mol. Recognit. 18(2):119-38 (2005). A yeast promoter is, for example, the ADH1 promoter (Ruohonen, et al., J Biotechnol. 1995 May 1; 39(3):193-203), or a constitutively active version thereof (e.g., the first 700 bp). Some embodiments include a terminator, such as the rp141b terminator resulted in the highest GFP expression out of over 5300 yeast promoters tested (Yamaishi, et al., ACS Synth. Biol., 2013, 2 (6), pp 337-347). Other suitable promoters, terminators, and vectors for yeast and yeast transformation protocols are well known in the art.

In some embodiments, the host cells are eukaryotic cells. For example, mammalian and insect host cell culture systems well known in the art can also be employed to express functionalized tRNA and mRNA for producing hybrid polypeptides. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are well known in the art.

The host organism can be a genomically recoded organism “GRO.” Typically, the GRO is a bacterial strain, for example, an E. coli bacterial strain, wherein a codon has been replaced by a synonymous codon. Because there are 64 possible 3-base codons, but only 20 canonical amino acids (plus stop codons), some amino acids are coded for by 2, 3, 4, 5, or 6 different codons (referred to herein as “synonymous codons”). In a GRO, most or all of the iterations of a particular codon are replaced with a synonymous codon. The precursor strain of the GRO is recoded such that at a least one codon is completely absent from the genome. Removal of a codon from the precursor GRO allows reintroduction of the deleted codon in, for example, a heterologous mRNA of interest. As discussed in more detail below, the reintroduced codon is typically dedicated to a non-standard amino acid, which in the presence of the appropriate translation machinery, can be incorporated in the nascent peptide chain during translation of the mRNA.

Different organisms often show particular preferences for one of the several codons that encode the same amino acid, and some codons are considered rare or infrequent. Preferably, the replaced codon is one that is rare or infrequent in the genome. The replaced codon can be one that codes for an amino acid (i.e., a sense codon) or a translation termination codon (i.e., a stop codon). GRO that are suitable for use as host or parental strains for the disclosed systems and methods are known in the art, or can be constructed using known methods. See, for example, Isaacs, et al., Science, 333, 348-53 (2011), Lajoie, et al., Science 342, 357-60 (2013), Lajoie, et al., Science, 342, 361-363 (2013).

Preferably, the replaced codon is one that codes for a rare stop codon. In a particular embodiment, the GRO is one in which all instances of the UAG (TAG) codon have been removed and replaced by another stop codon (e.g., TAA, TGA), and preferably wherein release factor 1 (RF1; terminates translation at UAG and UAA) has also been deleted, eliminating translational termination at UAG codons (Lajoie, et al., Science 342, 357-60 (2013)). In a particular embodiment, the host or precursor GRO is C321.Δ A [321 UAG→UAA conversions and deletion of prfA (encodes RF1)](genome sequence at GenBank accession CP006698). This GRO allows the reintroduction of UAG codons in a heterologous mRNA, along with orthogonal translation machinery, to permit efficient and site specific incorporation of the functional molecule into proteins encoded by the recoded gene of interest. That is, UAG has been transformed from a nonsense codon (terminates translation) to a sense codon (incorporates the functional molecule of choice), provided the appropriate translation machinery is present. UAG is a preferred codon for recoding because it is the rarest codon in Escherichia coli MG1655 (321 known instances) and a rich collection of translation machinery capable of incorporating non-standard amino acids has been developed for UAG (Liu and Schultz, Annu. Rev. Biochem., 79:413-44 (2010)).

Stop codons include TAG (UAG), TAA (UAA), and TGA (UGA). Although recoding to UAG (TAG) is discussed in more detail above, it will be appreciated that either of the other stop codons (or any sense codon) can be recoded using the same strategy. Accordingly, in some embodiments, a sense codon is reassigned, e.g., AGG or AGA to CGG, CGA, CGC, or CGG (arginine), e.g., as the principles can be extended to any set of synonymous or even non-synonymous codons, that are coding or non-coding. Similarly, the cognate translation machinery can be removed/mutated/deleted to remove natural codon function (UAG—RF1, UGA—RF2). The orthogonal translation system, particularly the antisense codon of the tRNA, can be designed to match the reassigned codon.

GRO can have two, three, or more codons replaced with a synonymous or non-synonymous codon. Such GRO allow for reintroduction of the two, three, or more deleted codons in one or more recoded genes of interest, each dedicated to a different non-standard amino acid. Such GRO can be used in combination with the appropriate orthogonal translation machinery to produce polypeptides having two, three, or more different non-standard amino acids.

Another host cell system for the use of codons containing unnatural bases is E. coli expressing Phaeodactylum tricornutum nucleotide triphosphate transporters as reported (Malyshev, et al., Nature, 509:385-388 (2014)).

### IV. Purifying Compounds Containing Functional Molecules

Proteins or polypeptides containing functional molecules can be purified, either partially or substantially to homogeneity, according to standard procedures known to and used by those of skill in the art including, but not limited to, ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, and gel electrophoresis. Protein refolding steps can be used, as desired, in making correctly folded mature proteins. High performance liquid chromatography (HPLC), affinity chromatography or other suitable methods can be employed in final purification steps where high purity is desired. In one embodiment, antibodies made against proteins containing the functional molecule are used as purification reagents, e.g., for affinity-based purification of proteins containing the functional molecule.

In some embodiments, hybrid polypeptides can be engineered to contain an additional domain containing amino acid sequence that allows the polypeptides to be captured onto an affinity matrix. For example, an Fe-containing polypeptide in a cell culture supernatant or a cytoplasmic extract can be isolated using a protein A column. In addition, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify polypeptides. Polypeptides can additionally be engineered to contain a secretory signal (if there is not a secretory signal already present) that causes the protein to be secreted by the cells in which it is produced. The secreted proteins can then conveniently be isolated from the cell media.

Once purified, partially or to homogeneity, as desired, the polypeptides may be used as assay components, therapeutic reagents, immunogens for antibody production, etc.

Those of skill in the art will recognize that, after synthesis, expression and/or purification, proteins can possess conformations different from the desired conformations of the relevant polypeptides. For example, polypeptides produced by prokaryotic systems often are optimized by exposure to chaotropic agents to achieve proper folding. During purification from lysates derived from E. coli, the expressed protein is optionally denatured and then renatured. This can be accomplished by solubilizing the proteins in a chaotropic agent such as guanidine HCl.

It is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Refolding reagents can be flowed or otherwise moved into contact with the one or more polypeptide or other expression product, or vice-versa.

### V. Kits

Kits for producing functinalized polypeptides are also provided. For example, a kit for producing a protein that contains one or more dipeptides, or non-standard-, non-natural-, or non-α-amino acids in a cell is provided, where the kit includes a polynucleotide sequence encoding wildtype, mutant, or engineered ribosomes (or a ribosomal rRNA thereof), tRNAs, or synthetases or a combination thereof. In one embodiment, the kit further includes one or more functional molecule precursors. In another embodiment, the kit includes a polynucleotide sequence encoding one or more translation system components. Any of the kits can include instructional materials for producing the protein.

### VI. Exemplary Applications

The materials produced herein can be used to generate artificial proteins with prescribed half-lives or immunogenicity, defined intracellular targeting pathways, or unique bioactivity. They can be used to generate libraries of molecules that can be screened for new materials or bioactivity.

The disclosed compositions and methods can be further understood through the following numbered paragraphs.

The disclosed compositions and methods can be further understood through the following numbered paragraphs.

1. A functionalized tRNA comprising a functional molecule comprising or consisting of a benzoic acid or benzoic acid derivative acylated to the 3′ nucleotide of a natural or engineered tRNA or tRNA-like molecule.

2. The functionalized tRNA of paragraph 1 having a structure of Formula II, Formula II′, or Formula II″:

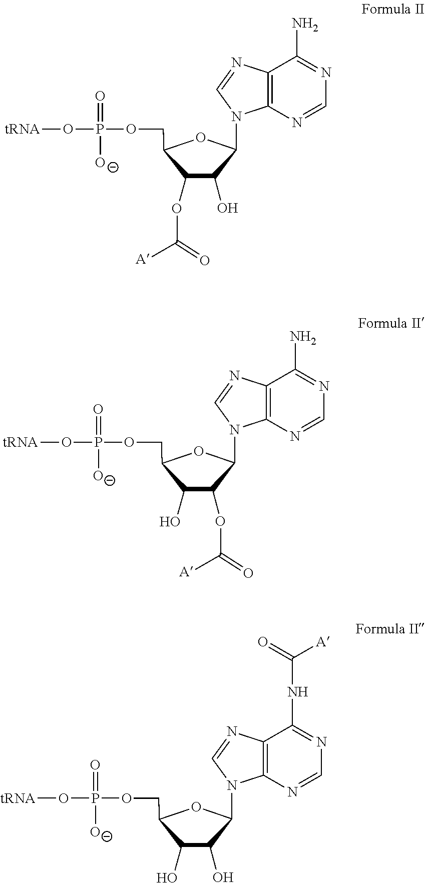

wherein A′ is an unsubstituted aryl group, a substituted aryl group, an unsubstituted heteroaryl group, or a substituted heteroaryl group,

the adenine of Formula II, Formula II′, or Formula II″ is the 3′ nucleotide of the tRNA, and the adenine of Formula II, Formula II′, or Formula II″ can be adenine, cytosine, guanine, thymine, or uracil, more particularly the adenine can be adenine, cytosine, guanine, or uracil in Formula II or Formula II′, or adenine, cytosine, or guanine in Formula II″, and

the “tRNA” of Formula II, Formula II′, or Formula II″ comprises the remaining nucleotides of the functionalized tRNA.

3. The functionalized tRNA of paragraphs 1 or 2 wherein the tRNA is not anthraniloyl-tRNA.

4. The functionalized tRNA of any one of paragraphs 1-3, wherein A′ is an unsubstituted aryl group or a substituted aryl group.

5. The functionalized tRNA of any one of paragraphs 1-4, wherein A′ is a substituted aryl group.

6. The functionalized tRNA of any one of paragraphs 1-5 having a structure of Formula III, Formula III′, or Formula III″:

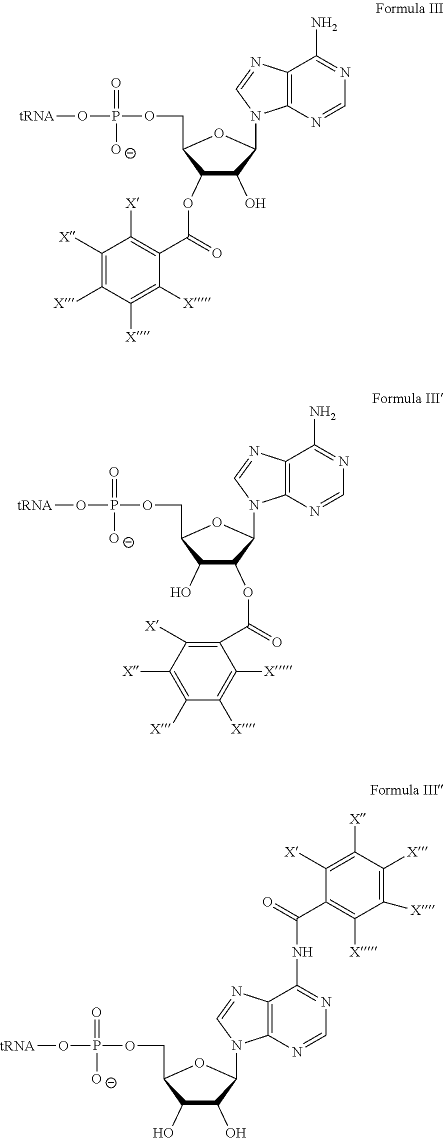

wherein X′, X″, X′″, X″″, and X′″″ are independently a hydrogen atom, a deuterium atom, a tritium atom, or a halogen atom selected from fluorine, chlorine, bromine, and iodine,

the adenine of Formula III, Formula III′, or Formula III″ is the 3′ nucleotide of the tRNA, and the adenine of Formula III, Formula III′, or Formula III″ can be adenine, cytosine, guanine, thymine, or uracil, more particularly the adenine can be adenine, cytosine, guanine, or uracil in Formula III or Formula III′, or adenine, cytosine, or guanine in Formula III″, and

the “tRNA” of Formula III, Formula III′, or Formula III″ comprises the remaining nucleotides of the functionalized tRNA.

7. The functionalized tRNA of paragraph 6, wherein X′ is fluorine.

8. The functionalized tRNA of any one of paragraphs 1-5 having a structure of Formula IV, Formula IV′, or Formula IV″:

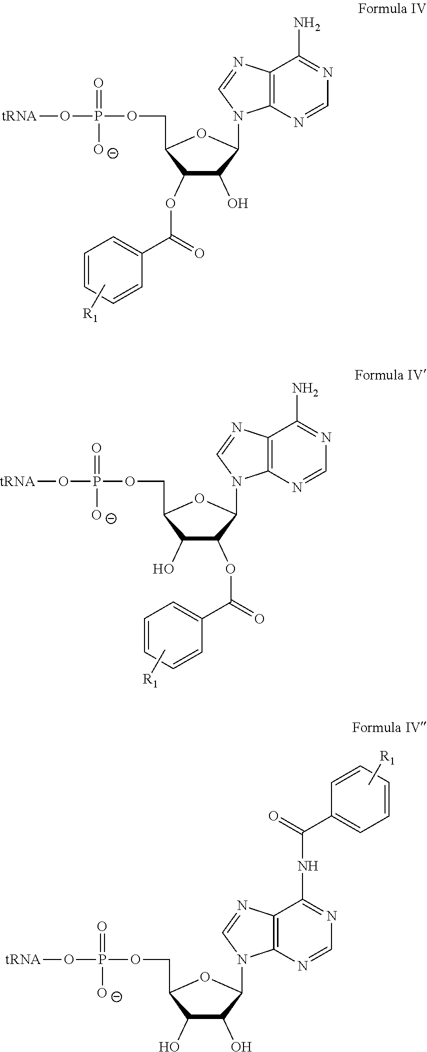

wherein R₁ is

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,

the adenine of Formula IV, Formula IV′, or Formula IV″ is the 3′ nucleotide of the tRNA, and the adenine of Formula IV, Formula IV′, or Formula IV″ can be adenine, cytosine, guanine, thymine, or uracil, more particularly the adenine can be adenine, cytosine, guanine, or uracil in Formula IV or Formula IV′, or adenine, cytosine, or guanine in Formula IV″, and

the “tRNA” of Formula IV, Formula IV′, or Formula IV″ comprises the remaining nucleotides of the functionalized tRNA.

9. The functionalized tRNA of paragraph 8, wherein R₁ is not a hydrogen bond donor.

10. The functionalized tRNA of paragraph 1, wherein A′ is an unsubstituted heteroaryl group or a substituted hereoaryl group.

11. The functionalized tRNA of paragraph 1 or paragraph 10, wherein A′ is a substituted heteroaryl group.

12. The functionalized tRNA of any one of paragraph 1, paragraph 10, or paragraph 11 having a structure of Formula V, Formula V′, or Formula V″:

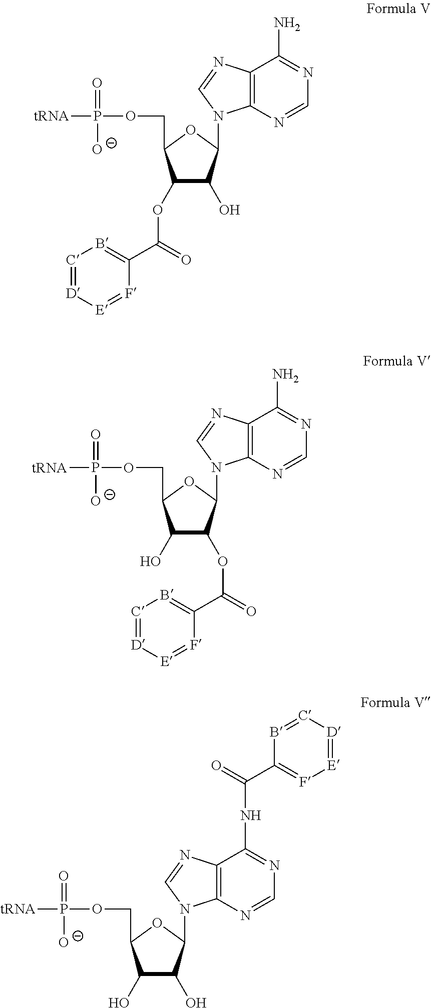

where B′, C′, D′, E′, and F′ are independently C—R₁ or a nitrogen atom;

where at least one of B′, C′, D′, E′, and F′ is a nitrogen atom;

wherein R₁ is

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,

the adenine of Formula V, Formula V′, or Formula V″ is the 3′ nucleotide of the tRNA, and the adenine of Formula V, Formula V′, or Formula V″ can be adenine, cytosine, guanine, thymine, or uracil, more particularly the adenine can be adenine, cytosine, guanine, or uracil in Formula V or Formula V′, or adenine, cytosine, or guanine in Formula V″, and

the “tRNA” of Formula V, Formula V′, or Formula V″ comprises the remaining nucleotides of the functionalized tRNA.

13. A functionalized tRNA comprising a functional molecule comprising or consisting of a malonic acid or malonic acid derivative acylated to the 3′ nucleotide of a natural or engineered tRNA or tRNA-like molecule.

14. The functionalized tRNA of paragraph 13 having a structure of Formula XIV, XIV′, or XIV″:

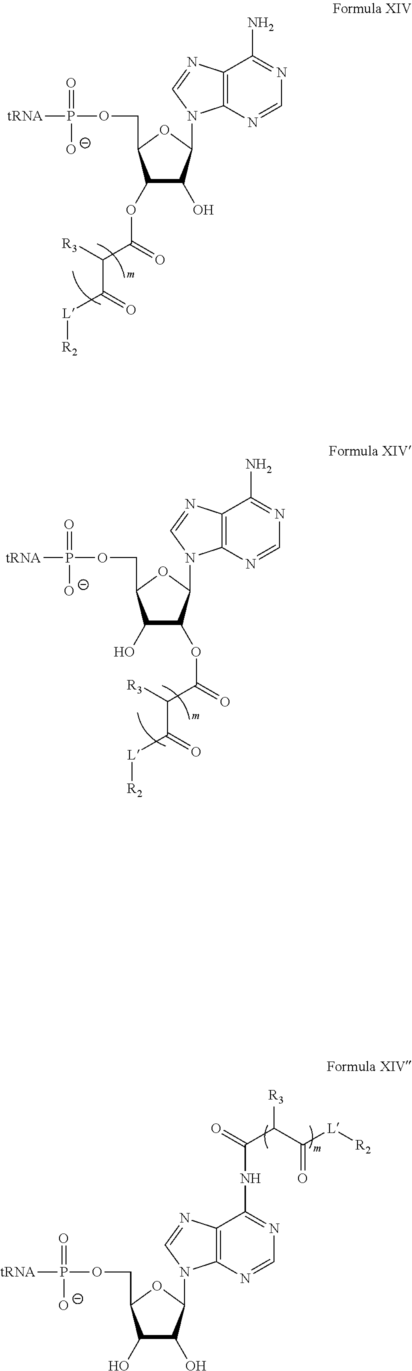

(a) wherein L′ is an oxygen atom, a nitrogen atom, or a sulfur atom;

(b) wherein m is an integer between 1 and 10 inclusive; and

(c) wherein R₂ and each R₃ are independently:

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,

the adenine of Formula XIV, Formula XIV′, or Formula XIV″ is the 3′ nucleotide of the tRNA, and the adenine of Formula XIV, Formula XIV′, or Formula XIV″ can be adenine, cytosine, guanine, thymine, or uracil, more particularly the adenine can be adenine, cytosine, guanine, or uracil in Formula XIV or Formula XIV′, or adenine, cytosine, or guanine in Formula XIV″, and

the “tRNA” of Formula XIV, Formula XIV′, or Formula XIV″ comprises the remaining nucleotides of the functionalized tRNA.

15. The functionalized tRNA of paragraph 14, wherein R₃ is a hydrogen atom and m is 1.

16. The functionalized tRNA of paragraph 14 or paragraph 15, wherein R₂ is a substituted aryl group.

17. The functionalized tRNA of any one of paragraphs 1-16, wherein the functional molecule does not comprise an amino acid.

18. The functionalized tRNA of paragraph 1 having a structure of Formula XII:

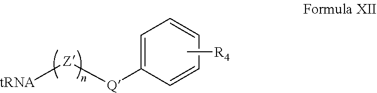

(a) wherein each Z′ is an amino acid;

(b) wherein n is an integer between 1 and 4 inclusive;

(c) wherein Q′ is an amide group or an ester group; and

(d) wherein R₄ comprises an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

19. The functionalized tRNA of paragraph 18, wherein R₄ comprises a primary amine.

20. The functionalized tRNA of any one of paragraphs 1-19, wherein the tRNA is an initiator tRNA.

21. The functionalized tRNA of any one of paragraphs 1-19, wherein the tRNA is an elongator tRNA.

22. The functionalized tRNA of any one of paragraphs 1-21, wherein the tRNA is a naturally occurring tRNA.

23. The functionalized tRNA of paragraph 22, wherein the tRNA is a bacterial tRNA.

24. The functionalized tRNA of any one of paragraphs 1-21, wherein the tRNA is non-naturally occurring tRNA.

25. The functionalized tRNA of any one of paragraphs 1-21, wherein the tRNA is a suppressor tRNA.

26. A method of making a functionalized polypeptide comprising providing or expressing a messenger RNA (mRNA) encoding the target polypeptide in a translation system comprising the functionalized tRNA of any one of paragraphs 1-25,

wherein the functionalized tRNA recognizes at least one codon such that functional molecule is incorporated into a polypeptide during translation.

27. The method of paragraph 26, wherein incorporation of the functional molecule occurs in vitro in a cell-free translation system.

28. The method of paragraph 26, wherein incorporation of the functional molecule occurs in vivo in a host cell.

29. The method of paragraph 28, wherein the host cell is a prokaryote.

30. The method of paragraphs 28 or 29, wherein a polynucleotide encoding the tRNA and a flexizyme capable of acylating the tRNA with the functional molecule are expressed in the host cell.

31. The method of any one of paragraphs 28-30 wherein the host cell is a genomically recoded organism (GRO).

32. A functionalized polypeptide comprising two or more amino acids and at least one functional molecule comprising or consisting of a benzoic acid or benzoic acid derivative; or a malonic acid or malonic acid derivative.

33. The functionalized polypeptide of paragraph 32 comprising the functional molecule at the N-terminus, the C-terminus, internally or a combination thereof.

34. The functionalized polypeptide of paragraphs 32 and 33 comprising functional molecules at the N-terminus, the C-terminus, and/or internally wherein two or more of the functional molecules are the same or different.

35. The functionalized polypeptide of any one of paragraphs 32-34 wherein the functional molecule(s) do not comprise an amino acid.

36. The functionalized polypeptide of any one of paragraphs 32-having a structure of Formula VI:

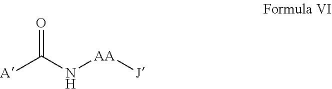

wherein A′ is an unsubstituted aryl group, a substituted aryl group, an unsubstituted heteroaryl group, or a substituted heteroaryl group;

wherein NH-AA is an amino acid which is linked to the functional molecule through a peptide bond; and

wherein J′ is one or more amino acids.

37. The functionalized polypeptide of paragraph 36, wherein A′ is an unsubstituted aryl group or a substituted aryl group.

38. The functionalized polypeptide of paragraphs 36 or 37, wherein A′ is a substituted aryl group.

39. The functionalized polypeptide of any one of paragraphs 36-38 having a structure of Formula VII:

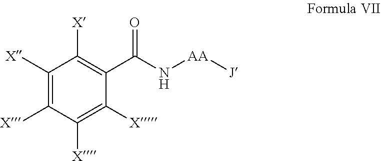

wherein NH-AA is an amino acid which is linked to the functional molecule through a peptide bond;

wherein J′ is one or more amino acids; and

wherein X′, X″, X′″, X″″, and X′″″ are independently a hydrogen atom, a deuterium atom, a tritium atom, or a halogen atom selected from fluorine, chlorine, bromine, and iodine.

40. The functionalized polypeptide of paragraph 39, wherein X′ is fluorine.

41. The functionalized polypeptide of any one of paragraphs 36-38 having a structure of Formula VIII:

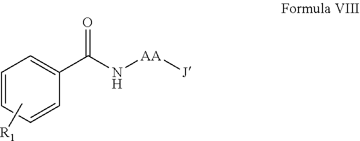

wherein NH-AA is an amino acid which is linked to the functional molecule through a peptide bond;

wherein J′ is one or more amino acids; and

wherein R₁ is

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

42. The functionalized polypeptide of paragraph 41, wherein R₁ is not a hydrogen bond donor.

43. The functionalized polypeptide of paragraph 36, wherein A′ is an unsubstituted heteroaryl group or a substituted hereoaryl group.

44. The functionalized polypeptide of paragraph 36 or paragraph 43, wherein A′ is a substituted heteroaryl group.

45. The functionalized polypeptide of any one of paragraphs 36, paragraph 43, or paragraph 44 having a structure of Formula IX:

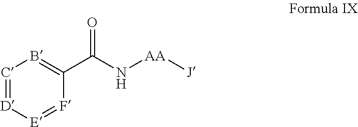

wherein NH-AA is an amino acid which is linked to the functional molecule through a peptide bond;

wherein J′ is one or more amino acids;

where B′, C′, D′, E′, and F′ are independently C—R₁ or a nitrogen atom;

where at least one of B′, C′, D′, E′, and F′ is a nitrogen atom; and

wherein R₁ is

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

46. The functionalized polypeptide of any one of paragraphs 32-having a structure of Formula XI:

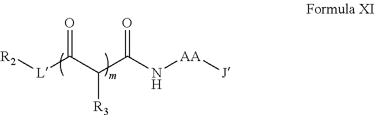

(a) wherein L′ is an oxygen atom, a nitrogen atom, or a sulfur atom;

(b) wherein m is an integer between 1 and 10 inclusive; and

(c) wherein R₂ and each R₃ are independently:

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

47. The functionalized polypeptide of paragraph 46, wherein R₃ is a hydrogen atom and m is 1.

48. The functionalized polypeptide of paragraph 46 or paragraph 47, wherein R₂ is a substituted aryl group.

49. The functionalized polypeptide of any one of paragraphs 32-34 having a structure of Formula XII′:

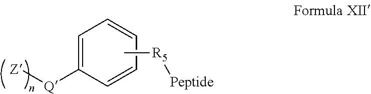

(a) wherein each Z′ is an amino acid;

(b) wherein n is an integer between 1 and 4 inclusive;

(c) wherein Q′ is an amide group or an ester group; and

(d) wherein R₅ comprises a secondary amino group optionally containing a substituent at the amino nitrogen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group.

50. A functionalized polypeptide made according to the method of any one of paragraphs 26-31.

51. A functionalized polypeptide of any one of paragraphs 32-49 made according to the method of any one of paragraphs 23-31.

The present invention will be further understood by reference to the following non-limiting examples.

### EXAMPLES

The Examples below illustrate that wild type E. coli ribosomes accept and elongate pre-charged initiator tRNAs acylated with multiple benzoic acids, including aramid precursors, as well as malonyl (α,β-diketo) substrates to generate a diverse set of aramid-peptide and polyketide-peptide hybrid molecules.

### Example 1: Synthetic Schemes

### 1. Commercial Benzoic Acids to CME Esters (1-3, 8-13)

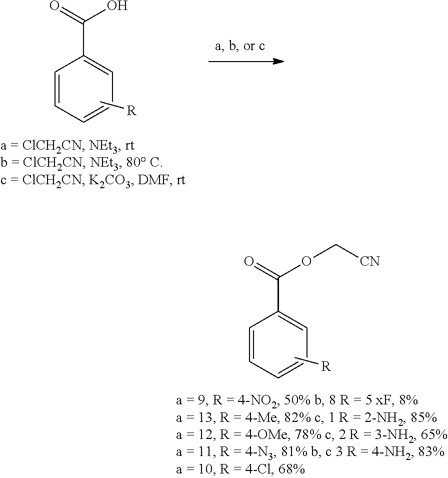

### 2. Cyanomethyl 4-aminonicotinate (6)

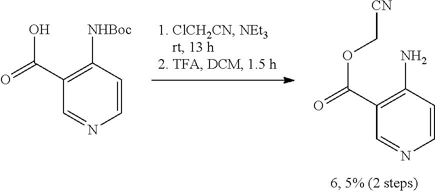 [SMILES: CC(C)(C)OC(=O)Nc1ccncc1C(=O)O ; N#CCOC(=O)c1cnccc1N]

### 3. 2 and 4 Amino DBE Esters (4 and 5)

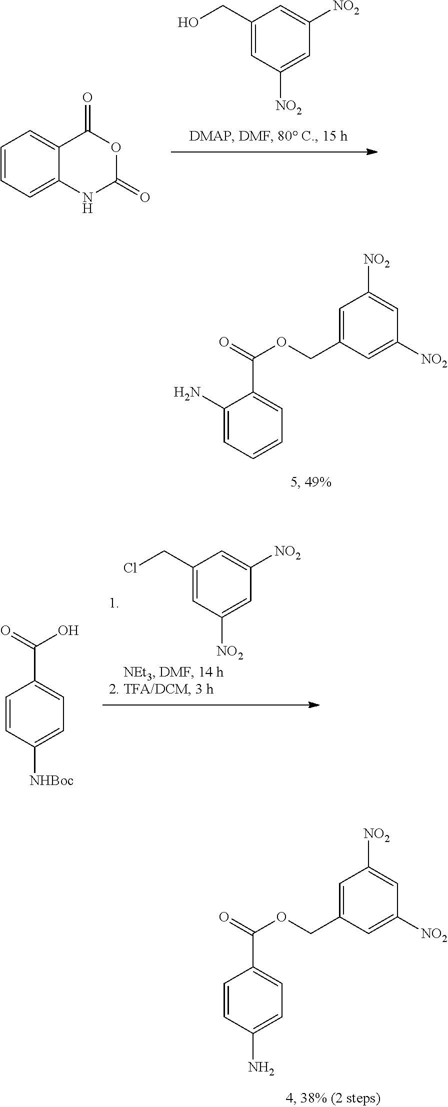 [SMILES: O=c1[nH]c2ccccc2c(=O)o1 ; O=[N+]([O-])c1cc(CO)cc([N+](=O)[O-])c1 ; Nc1ccccc1C(=O)OCc1cc([N+](=O)[O-])cc([N+](=O)[O-])c1 ; CC(C)(C)OC(=O)Nc1ccc(C(=O)O)cc1 ; O=[N+]([O-])c1cc(CCl)cc([N+](=O)[O-])c1 ; Nc1ccc(C(=O)OCc2cc([N+](=O)[O-])cc([N+](=O)[O-])c2)cc1]

### 4. Cyanomethyl 4-(azidomethyl)benzoate (14)

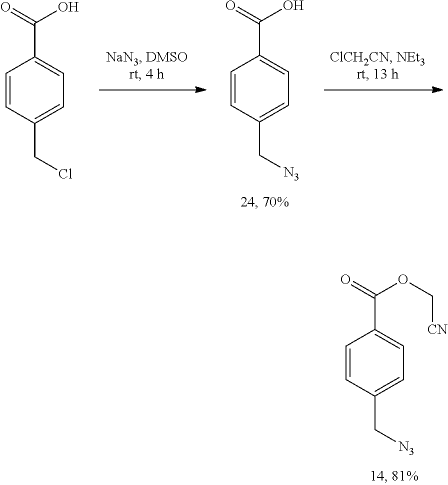 [SMILES: O=C(O)c1ccc(CCl)cc1 ; [N-]=[N+]=NCc1ccc(C(=O)O)cc1 ; N#CCOC(=O)c1ccc(CN=[N+]=[N-])cc1]

### 5. Cyanomethyl 4-carboxy and (hydroxymethyl)benzoates (17)

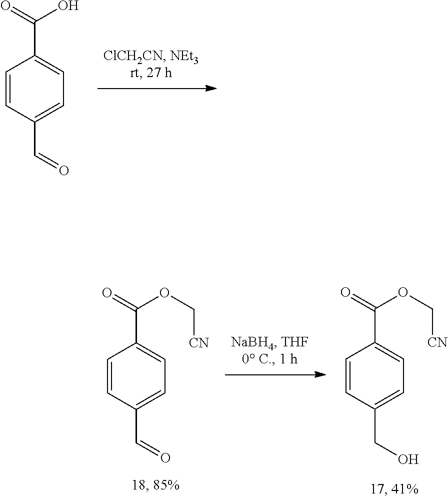 [SMILES: O=Cc1ccc(C(=O)O)cc1 ; N#CCOC(=O)c1ccc(C=O)cc1 ; N#CCOC(=O)c1ccc(CO)cc1]

### 6. Cyanomethyl 4-(methylamino)benzoate hydrochloride (15)

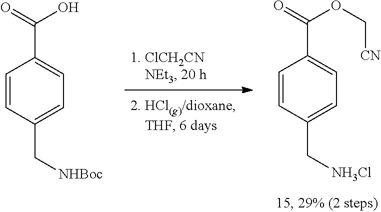

### 7. Cyanomethyl 4-hydroxybenzoate (16)

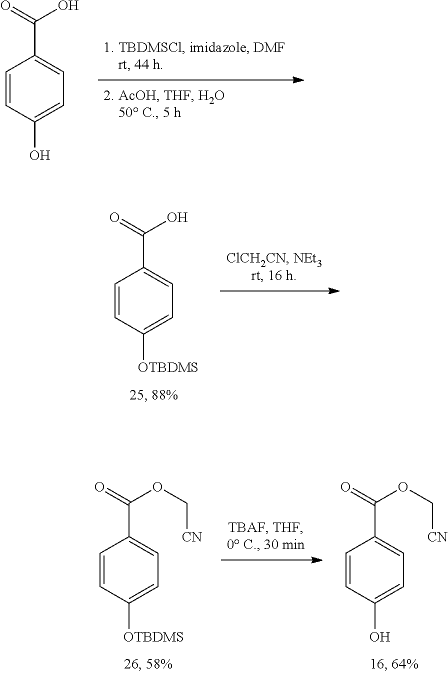 [SMILES: O=C(O)c1ccc(O)cc1 ; CC(C)(C)[Si](C)(C)Oc1ccc(C(=O)O)cc1 ; CC(C)(C)[Si](C)(C)Oc1ccc(C(=O)OCC#N)cc1 ; N#CCOC(=O)c1ccc(O)cc1]

### 8. Cyanomethyl (2-nitrobenzyl) malonate (22)

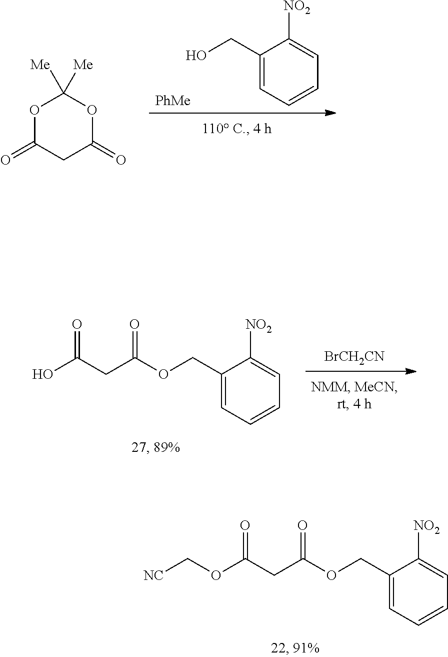

### 9. N-Formylmethionyl 3,5-dintrobenzyl methyl ester (28)

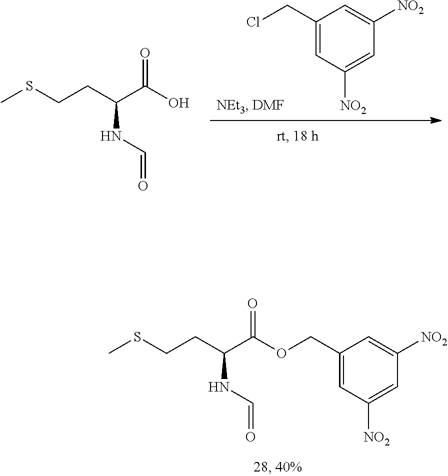

### 10. 4-Chlorobenzyl mercaptan malonate (19)

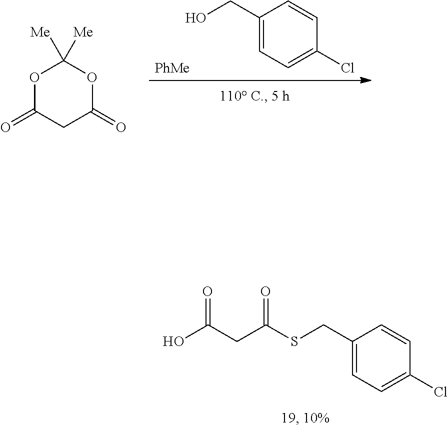 [SMILES: CC1(C)OC(=O)CC(=O)O1 ; "PhMe" ; OCc1ccc(Cl)cc1 ; O=C(O)CC(=O)SCc1ccc(Cl)cc1]

### 11. 3,5-Dinitrobenzyl methyl malonate (23)

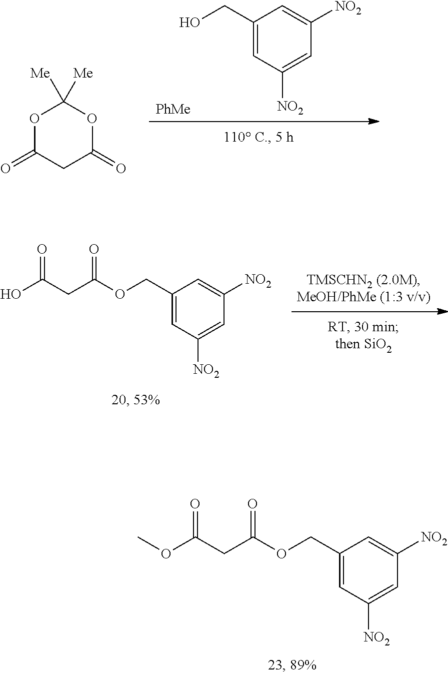 [SMILES: CC1(C)OC(=O)CC(=O)O1 ; O=[N+]([O-])c1cc(CO)cc([N+](=O)[O-])c1 ; "PhMe" ; O=C(O)CC(=O)OCc1cc([N+](=O)[O-])cc([N+](=O)[O-])c1 ; "TMSCHN2 (2.0M),MeOH/PhMe (1:3 v/v)" ; COC(=O)CC(=O)OCc1cc([N+](=O)[O-])cc([N+](=O)[O-])c1]

### 12. 2-benzyl-3-((3,5-dinitrobenzyl)oxy)-3-oxopropanoic acid (21)

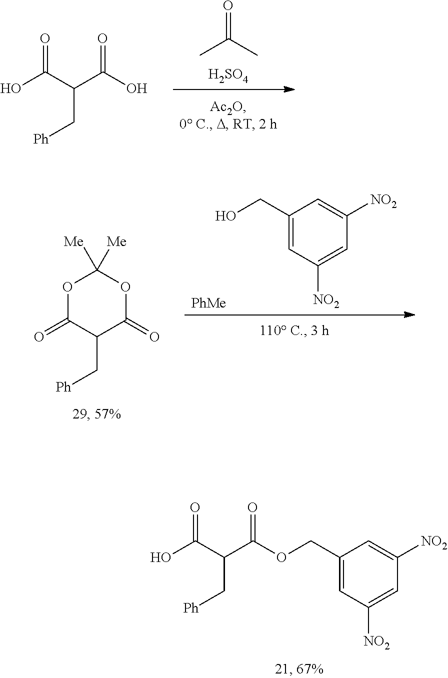

Synthetic Protocols

General Notes: All reagents and solvents were used as received from commercial suppliers, unless indicated otherwise. Anhydrous methanol was purchased from a commercial supplier. Other anhydrous solvents were obtained from a solvent drying system and collected fresh prior to every reaction. All reactions were carried out without exclusion of air or moisture unless otherwise stated. Room temperature is considered 20-23° C. Stirring was achieved with Teflon coated magnetic stir bars. TLC was performed on glass backed silica gel plates (median pore size 60 Å) and visualized using UV light at 254 nm, or staining with KMnO₄ or ninhydrin. Column chromatography was performed on an Isco Teledyne Combiflash RF instrument using pre-packed Redi-sep silica gel cartridges (particle diameter 35-70 μM, pore diameter 60 Å); eluents are given in brackets. MS characterization was carried out on an Agilent 6530 QTOF AJS-ESI (G6230BAR). The following parameters were used: Fragmentor voltage 175 V, Gas temperature 300° C., Gas flow 12 L/min, Sheath gas temperature 350° C., Sheath gas flow 11 L/min, Nebulizer pressure 35 psi, skimmer voltage 65 V, Vcap 3500 V, 1 spectra/s in either positive or negative mode. ¹H and ¹³C NMR spectra were recorded on Agilent DDR2 400, 500 or 600 MHz spectrometers (as specified in the characterization data) at 298 K, and calibrated by using the residual peak of the solvent as the internal standard (CDCl₃: δ_{H}=7.26 ppm; δ_{C}=77.16 ppm; DMSO-d₆: δ_{H}=2.50 ppm; δ_{C}=39.52 ppm; CD₃OD: δ_{H}=3.31 ppm; δ_{C}=49.00 ppm, acetone-d₆ δ_{H}=2.05 ppm; δ_{C}=29.84 ppm). All coupling constants are recorded in Hz. ¹⁹F-HSQCAD experiments were used to identify ¹³C signals for polyfluorinated substrate 8—peaks identified this way are marked with an asterisk* in the characterization data. NMR spectra were processed with MestReNova v10.0.1-14719 software using the baseline and phasing correction features. Multiplicities and coupling constants were calculated using the multiplet analysis feature with manual intervention as necessary. Probable AA‘BB’ systems from 1,4 disubstituted phenyl rings were reported as doublets.

### Cyanomethyl 4-nitrobenzoate (9)

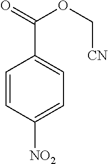 [SMILES: N#CCOC(=O)c1ccc([N+](=O)[O-])cc1]

4-Nitrobenzoic acid (530 mg, 3.16 mmol, 1.00 equiv.) was suspended in a solution of chloroacetonitrile (1.00 mL, 15.8 mmol, 4.40 equiv.) and triethylamine (1.0 mL, 7.2 mmol, 2.3 equiv.) The solution was stirred for 2 h and partitioned with EtOAc and 0.5 M HCl_{(aq)}. The organic layer was then sequentially washed with 0.2 M NaHCO_{3(aq)}, water, and brine. The organic layer was dried over MgSO₄, filtered, and loaded onto SiO₂ by removing the solvent under reduced pressure. Purified by chromatography (0-50% EtOAc in hexanes) to give cyanomethyl 9 as a white powder (300 mg, 50%). ¹H NMR (500 MHz, DMSO-d₆) δ 8.37 (d, J=8.9 Hz, 2H), 8.23 (d, J=8.9 Hz, 2H), 5.29 (s, 2H). ¹³C NMR (126 MHz, DMSO-d₆) δ 163.3, 150.7, 133.4, 131.1, 124.1, 115.7, 50.5. HR-ESI-MS [M−H]⁻: calculated for C₉H₆N₂O₄, m/z 205.0255, found m/z 205.0254.

### Cyanomethyl 4-methylbenzoate (13)

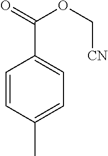

4-Methylbenzoic acid (50 mg, 0.37 mmol, 1.0 equiv.) was suspended in chloroacetonitrile (234 μL, 3.70 mmol, 10.0 equiv.), followed by addition of triethylamine (103 μL, 0.739 mmol, 2.00 equiv.). The solution was stirred at rt for 12 h, then partitioned between EtOAc and water. Sat. Na₂SO_{4(aq)} was added to the aqueous layer, which was then re-extracted (EtOAc). The combined organics were dried over MgSO₄, filtered, and loaded onto SiO₂ by removing the solvent under reduced pressure. Purified by chromatography (0-70% EtOAc/hexanes) to give cyanomethyl ester 13 as a colorless oil (53 mg, 82%). ¹H NMR (500 MHz, CDCl₃) δ 7.94 (d, J=8.3 Hz, 2H), 7.27 (d, J=8.1 Hz, 2H), 4.94 (s, 2H), 2.42 (s, 3H). ¹³C NMR (126 MHz, CDCl₃) δ 165.1, 145.2, 130.1, 129.5, 125.2, 114.7, 48.8, 21.9. HR-ESI-MS [M+H]⁺: calculated for C₁₀H₉NO₂, M/z 176.0706, found m/z 176.0702.

### Cyanomethyl 4-chlorobenzoate (10)

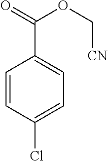 [SMILES: N#CCOC(=O)c1ccc(Cl)cc1]

4-Chlorobenzoic acid (50 mg, 0.32 mmol, 1.0 equiv.) was suspended in chloroacetonitrile (200 μL, 3.16 mmol, 9.88 equiv.) followed by addition of triethylamine (90 μL, 0.65 mmol, 2.0 equiv.). The solution was stirred at rt for 12 h, then partitioned between EtOAc and water. Sat. Na₂SO_{4(aq)} was added to the aqueous, which was then re-extracted (EtOAc), then the combined organics were dried over MgSO₄, filtered, and loaded onto SiO₂ by removing the solvent under reduced pressure. Purified by chromatography (0-100% EtOAc/hexanes) to give cyanomethyl ester 10 as a colorless oil (43 mg, 68%). ¹H NMR (500 MHz, CDCl₃) δ 7.99 (d, J=8.5 Hz, 2H), 7.46 (d, J=8.5 Hz, 2H), 4.96 (s, 2H). ¹³C NMR (126 MHz, CDCl₃) δ 164.3, 141.0, 131.5, 129.3, 126.4, 114.4, 49.1. HR-ESI-MS [M+H]⁺: calculated for C₉H₆ClNO₂, m/z, 196.0160, found m/z 196.0170.

### Cyanomethyl 4-methoxybenzoate (12)

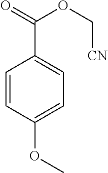

4-Methoxybenzoic acid (50 mg, 0.33 mmol, 1.0 equiv.) was suspended in chloroacetonitrile (200 μL, 3.16 mmol, 9.58 equiv.) followed by addition of triethylamine (92 μL, 0.66 mmol, 2.0 equiv.). The solution was stirred at rt for 12 h, then partitioned between EtOAc and water. Sat. Na₂SO_{4(aq)} was added to the aqueous, which was then re-extracted (EtOAc), and the combined organics were dried over MgSO₄, filtered, and loaded onto SiO₂ by removing the solvent under reduced pressure. Purified by chromatography (0-60% EtOAc/hexanes) to give cyanomethyl ester 12 as a colorless oil (49 mg, 78%). ¹H NMR (500 MHz, CDCl₃) δ 8.00 (d, J=8.9 Hz, 2H), 6.94 (d, J=8.9 Hz, 2H), 4.92 (s, 2H), 3.87 (s, 3H). ¹³C NMR (126 MHz, CDCl₃) δ 164.7, 164.4, 132.3, 120.2, 114.8, 114.1, 55.6, 48.7. HR-ESI-MS [M+H]⁺: calculated for C₁₀H₉NO₃, m/z 192.0655, found m/z 192.0657.

### Cyanomethyl 4-azidobenzoate (11)

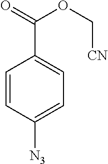 [SMILES: N#CCOC(=O)c1ccc(N=[N+]=[N-])cc1]

4-azidobenzoic acid (150 mg, 0.920 mmol, 1.00 equiv.) was suspended in chloroacetonitrile (291 μL, 4.60 mmol, 5.00 equiv.) followed by addition of triethylamine (257 μL, 1.84 mmol, 2.00 equiv.). The solution was stirred at rt for 13 h, then partitioned between EtOAc and water. The aqueous was then re-extracted (EtOAc), and the combined organics washed with brine and dried over MgSO₄. The solvent was removed under reduced pressure to give cyanomethyl ester 11 as a brown oil (151 mg, 81%). ¹H NMR (400 MHz, CDCl₃) δ 8.05 (d, J=8.7 Hz, 2H), 7.11 (d, J=8.7 Hz, 2H), 4.96 (s, 2H). ¹³C NMR (101 MHz, CDCl₃) δ 164.2, 146.2, 132.0, 124.3, 119.3, 114.5, 49.0. HR-ESI-MS [M+H−N₂]⁺: calculated for C₉H₆N₄O₂, m/z, 175.0502, found m/z 175.0502.

### 4-(Azidomethyl)benzoic acid (24)

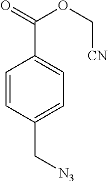 [SMILES: N#CCOC(=O)c1ccc(CN=[N+]=[N-])cc1]

4-(Chloromethyl)benzoic acid (500 mg, 2.93 mmol, 1.00 equiv.) and sodium azide (286 mg, 4.40 mmol, 1.50 equiv.) were dissolved in DMSO (3.5 mL) and the solution stirred at rt for 4 h. Water (10 mL) was added and the resulting precipitate vacuum filtered and the precipitate washed with water. The filter cake was dried under vacuum to give benzoic acid 24 as a beige powder (361 mg, 70%). ¹H NMR (400 MHz, DMSO-d₆) δ 13.00 (s, 1H), 7.96 (d, J=8.3 Hz, 2H), 7.49 (d, J=8.2 Hz, 2H), 4.57 (s, 2H). ¹³C NMR (126 MHz, DMSO-d₆) δ 167.0, 140.6, 130.4, 129.7, 128.3, 53.1. Compound previously reported in literature.⁵

### Cyanomethyl 4-(azidomethyl)benzoate (14)

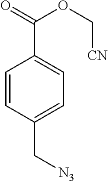 [SMILES: N#CCOC(=O)c1ccc(CN=[N+]=[N-])cc1]

4-(Azidomethyl)benzoic acid 24 (150 mg, 0.847 mmol, 1.00 equiv.) was suspended in chloroacetonitrile (291 μL, 4.60 mmol, 5.00 equiv.) followed by addition of triethylamine (257 μL, 1.84 mmol, 2.00 equiv.). The solution was stirred at rt for 13 h, then partitioned between EtOAc and water. The aqueous was then re-extracted (EtOAc), and the combined organics washed with brine and dried over MgSO₄. The solvent was removed under reduced pressure to give cyanomethyl ester 14 as a brown oil (151 mg, 83%). ¹H NMR (400 MHz, CDCl₃) δ 8.08 (d, J=8.3 Hz, 2H), 7.44 (d, J=8.2 Hz, 2H), 4.97 (s, 2H), 4.45 (s, 2H). ¹³C NMR (101 MHz, CDCl₃) δ 164.6, 142.0, 130.7, 128.3, 127.8, 114.5, 54.3, 49.0. HR-ESI-MS [M+H−N₂]⁺: calculated for C₁₀H₈N₄O₂, m/z, 189.0659, found m/z 189.0661.

### Cyanomethyl 4-2,3,4,5,6-pentafluorobenzoate (8)

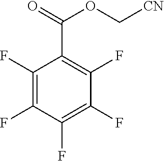 [SMILES: N#CCOC(=O)c1c(F)c(F)c(F)c(F)c1F]

Pentafluorobenzoic acid (150 mg, 0.707 mmol, 1.00 equiv.) was suspended in chloroacetonitrile (225 μL, 3.55 mmol, 5.00 equiv.) followed by addition of triethylamine (198 μL, 1.42 mmol, 2.00 equiv.). The solution was stirred at 80° C. for 13 h, then partitioned between EtOAc and water. The aqueous was then re-extracted (EtOAc), then the combined organics dried over MgSO₄ and loaded onto SiO₂ by removal of solvent under reduced pressure. Purified by chromatography (0-75% EtOAc in hexanes) to give cyanomethyl ester 8 as a brown oil (15 mg, 8%). ¹H NMR (400 MHz, CDCl₃) δ 5.01 (s, 2H). ¹⁹F NMR (376 MHz, CDCl₃) δ −136.02 (apparent dt, J=19.2, 6.1 Hz), −145.09 (tt, J=20.7, 6.1 Hz), −157.87-−160.94 (m). ¹³C NMR (151 MHz, CDCl₃) δ 146.2 (dm, J=264 Hz), 144.5 (dm, J=262 Hz), 138.0 (dm, J=256 Hz), 105.81 (apparent td, J=14.3, 4.2 Hz), 49.8. Note: The complexity resulting from ¹⁹F-¹³C coupling in polyfluorinated aromatics is difficult to report accurately, and varied approaches are used. The unusual ‘dm’ nomenclature was used. As shown in a recent manuscript (Ma, et al., Org Lett, 20, 2689-2692 (2018)) as this maximizes the data presented and most accurately represents the observed signals and the interactions from which they arise. HR-ESI-MS [M+H]⁺: calculated for C₉H₂F₅NO₂, m/z 252.0078, found m/z 252.0075.

### Cyanomethyl 4-aminonicotinate (6)

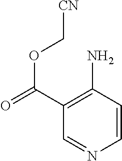 [SMILES: N#CCOC(=O)c1cnccc1N]

4-Boc-aminonicotinic acid (250 mg, 1.05 mmol, 1.00 equiv.) was suspended in chloroacetonitrile (332 μL, 5.25 mmol, 5.00 equiv.) followed by addition of triethylamine (292 μL, 1.84 mmol, 2.10 equiv.). The solution was stirred at rt for 13 h, then partitioned between EtOAc and water. The aqueous was then re-extracted (EtOAc), then the combined organics dried over MgSO₄. The solvent was removed under reduced pressure to give 87 mg of a mixture of cyanomethyl 4-Boc-aminonicotinate and the pyridine N-alkylation byproduct. This was redissolved in DCM (2 mL) and TFA (1 mL) added. After stirring for 90 mins at rt, the reaction was basified with NaHCO_{3(aq)} and extracted into DCM. The aqueous layer was re-extracted (DCM×1, EtOAc×1), and the combined organics dried over MgSO₄ and concentrated under reduced pressure. Purified by column chromatography (0-100% MeCN with 1% NEt₃/DCM) to give cyanomethyl ester 6 as a solid (9 mg, 5% over 2 steps). ¹H NMR (400 MHz, DMSO-d₆) δ 8.67 (s, 1H), 8.10 (d, J=6.0 Hz, 1H), 7.36 (s, br, 2H), 6.72 (d, J=6.0 Hz, 1H), 5.16 (s, 2H). ¹³C NMR (151 MHz, DMSO-d₆) δ 165.4, 155.4, 152.5, 152.0, 116.1, 110.9, 104.7, 49.1. HR-ESI-MS [M+H]⁺: calculated for CH₇N₃O₂, m/z, 178.0611, found m/z 178.0612.

### Cyanomethyl 4-(methylamino)benzoate hydrochloride (15)

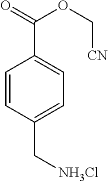

4-(N-bocmethylamino)benzoic acid (150 mg, 0.596 mmol, 1.00 equiv.) was suspended in a solution of chloroacetonitrile (190 μL, 3.00 mmol, 5.03 equiv.) and triethylamine (161 μL, 1.20 mmol, 2.01 equiv.) The solution was stirred for 20 h and partitioned with EtOAc and water. The organic layer was then re-extracted with EtOAc and the combined organics dried over MgSO₄, filtered, and the solvent removed under reduced pressure to give the cyanomethyl ester (177 mg). A portion of this material (100 mg, 0.344 mmol, 1.00 equiv.) was dissolved in THE (2.0 mL), and 4 M HCl_{(g)} (1.0 mL, 4.00 mmol, 11.6 equiv.) in dioxane added. The mixture was sealed and stirred overnight at rt, forming a white precipitate, then allowed to stand at rt for 5 days. The liquid was removed and the precipitate dried under a flow of N_{2(g)} to give hydrochloride salt 15 as a white solid (22 mg, 29% over 2 steps). ¹H NMR (500 MHz, DMSO-d₆) δ 8.58 (br s, 2H), 8.03 (d, J=8.2 Hz, 2H), 7.69 (d, J=8.1 Hz, 2H), 5.24 (s, 2H), 4.13 (s, 2H). ¹³C NMR (126 MHz, DMSO-d₆) δ 164.4, 140.4, 129.7, 129.4, 127.8, 116.0, 66.3, 49.9, 41.7. HR-ESI-MS [M+H−N₂]⁺: calculated for C₁₀H₁₀N₂O₂, m/z, 191.0815, found m/z 191.0815.

### 3,5-Dinitrobenzyl 2-aminobenzoate (5)

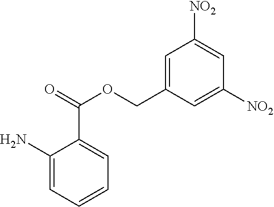 [SMILES: Nc1ccccc1C(=O)OCc1cc([N+](=O)[O-])cc([N+](=O)[O-])c1]

Isatoic anhydride (150 mg, 0.920 mmol, 1.00 equiv.), 3,5-dinitrobenzyl alcohol (216 mg, 1.09 mmol, 1.20 equiv.) and DMAP (11 mg, 0.09 mmol, 0.10 equiv.) were combined in DMF (1.0 mL) and heated to 80° C. for 15 h. On cooling the mixture solidified, so 1 mL EtOAc was added and the mixture was heated until a stirring suspension was achieved, then allowed to cool to rt. After stirring for 1 h at rt the mixture was filtered and the filter cake washed with EtOAc, then dried under vacuum to give ester 5 as a yellow solid (135 mg, 49%). ¹H NMR (400 MHz, DMSO-d₆) δ 8.80 (t, J=2.2 Hz, 1H, CH_{A}r), 8.75 (d, J=2.1 Hz, 2H), 7.78 (dd, J=8.1, 1.6 Hz, 1H), 7.29 (ddd, J=8.5, 6.9, 1.7 Hz, 1H), 6.80 (dd, J=8.4, 1.2 Hz, 1H), 6.70 (s, br, 2H), 6.56 (ddd, J=8.1, 6.9, 1.2 Hz, 1H), 5.54 (s, 2H). ¹³C NMR (151 MHz, DMSO-d₆) δ 166.8, 151.7, 148.1, 141.0, 134.5, 130.6, 128.3, 118.1, 116.7, 114.9, 107.9, 63.5. HR-ESI-MS [M+H]⁺: calculated for C₁₄H₁₁N₃O₆, m/z, 318.0721, found m/z 318.0727.

### 3,5-Dinitrobenzyl 4-aminobenzoate (4)

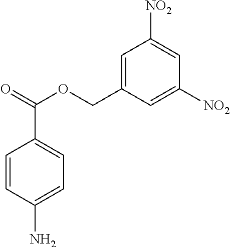 [SMILES: Nc1ccc(C(=O)OCc2cc([N+](=O)[O-])cc([N+](=O)[O-])c2)cc1]

4-(Boc-amino)benzoic acid (225 mg, 0.948 mmol, 1.00 equiv.) and 2,4-dinitrobenzyl chloride (263 mg, 1.14 mmol, 1.20 equiv.) were combined in DMF (1.5 mL), followed by addition of triethylamine (263 μL, 1.90 mmol, 2.00 equiv.). Stirred at rt for 14 h, then partitioned between EtOAc and brine. The aqueous was re-extracted (EtOAc) and the combined organics washed with 5% LiCl_{(aq)} and dried over MgSO₄. The solvent was removed under reduced pressure, then the residue re-dissolved in DCM (2.0 mL) and 0.60 mL TFA (0.60 mL, 7.8 mmol, 8.2 equiv.) added. After stirring at rt for 3 h, the reaction mixture was partitioned between EtOAc and NaHCO_{3(aq)}.—due to low solubility the organic layer was a suspension. Water, then sat. Na₂SO_{4(aq)} were added, and the aqueous re-extracted with EtOAc. The suspension from the combined organic layers were diluted with hexanes to approximately 2:1 EtOAc/hexanes by volume, then filtered under vacuum. The filtrate was dried over MgSO₄, then concentrated under reduced pressure to afford a residue that was dissolved in acetone and recombined with the filter cake. The solvent was removed under reduced pressure, then the residue heated to reflux in acetone (2 mL). The suspension was cooled to −20° C., then the solvent decanted and the residual solvent removed under reduced pressure to give dinitrobenzyl ester 4 as a yellow solid (115 mg, 38%). ¹H NMR (400 MHz, DMSO-d₆) δ 8.79 (s, 1H), 8.70 (d, J=2.1 Hz, 2H), 7.70 (d, J=8.7 Hz, 2H), 6.58 (d, J=8.7 Hz, 2H), 6.08 (s, 2H), 5.50 (s, 2H). ¹³C NMR (101 MHz, DMSO-d₆) δ 165.4, 154.0, 148.1, 141.3, 131.4, 128.1, 118.0, 114.8, 112.7, 63.3. HR-ESI-MS [M+H]⁺: calculated for C₁₄H₁₁N₃O₆, m/z, 318.0721, found m/z 318.0725.

### Cyanomethyl 4-formylbenzoate (18)

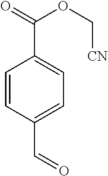 [SMILES: N#CCOC(=O)c1ccc(C=O)cc1]

4-formylbenzoic acid (300 mg, 2.00 mmol, 1.00 equiv.) was suspended in chloroacetonitrile (630 μL, 9.95 mmol, 4.98 equiv.) followed by addition of triethylamine (558 μL, 4.01 mmol, 2.01 equiv.). The solution was stirred at rt for 27 h, then partitioned between EtOAc and water. The aqueous was re-extracted (EtOAc), then the combined organics dried over MgSO₄ and loaded onto SiO₂ by removal of solvent under reduced pressure. Purified by chromatography (0-90% EtOAc in hexanes) to give cyanomethyl ester 18 as a white solid (322 mg, 85%). ¹H NMR (400 MHz, CDCl₃) δ 10.12 (s, 1H), 8.23 (d, J=8.3 Hz, 2H), 8.00 (d, J=8.4 Hz, 2H), 5.01 (s, 2H). ¹³C NMR (126 MHz, CDCl₃) δ 191.4, 164.1, 140.0, 132.8, 130.8, 129.8, 114.2, 49.4. HR-ESI-MS [M+H]⁺: calculated for C₁₀H₇NO₃, m/z 190.0499, found m/z 190.0496.

### Cyanomethyl 4-hydroxymethyl)benzoate (17)

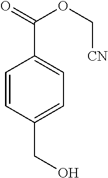 [SMILES: N#CCOC(=O)c1ccc(CO)cc1]

Cyanomethyl 4-formylbenzoate 18 (50 mg, 0.26 mmol, 1.0 equiv.) was dissolved in THE (1.0 mL) and cooled to 0° C., then sodium borohydride (14 mg, 0.40 mmol, 1.4 equiv.) was added. The solution was stirred at 0° C. and monitored by TLC. After 1 h the mixture was partitioned between 0.5 M HCl_{(aq)} and EtOAc, then the aqueous re-extracted (EtOAc). The combined organics were washed with brine, dried over MgSO₄ and loaded onto SiO₂ by removal of solvent under reduced pressure. Purified by chromatography (15-55% EtOAc in hexanes) to give cyanomethyl ester 17 as a white solid (21 mg, 41%). ¹H NMR (500 MHz, Acetone-d₆) δ 8.02 (d, J=8.3 Hz, 2H), 7.55 (d, J=8.1 Hz, 2H), 5.19 (s, 2H), 4.75 (d, J=5.0 Hz, 2H), 4.50 (t, J=5.5 Hz, 1H). ¹³C NMR (126 MHz, Acetone-d₆) δ 165.7, 150.2, 130.5, 127.7, 127.3, 116.2, 64.0, 50.0. HR-ESI-MS [M+H]⁺: calculated for C₁₀H₉NO₃, m/z 192.0655, found m/z 192.0653.

### Cyanomethyl 4-aminobenzoate (3)

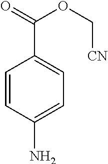 [SMILES: N#CCOC(=O)c1ccc(N)cc1]

4-Aminobenzoic acid (1.00 g, 7.29 mmol, 1.00 equiv.) was dissolved in DMF (5.0 mL) with potassium carbonate (2.02 g, 14.6 mmol, 2.00 equiv.), then chloroacetonitrile (452 μL, 7.14 mmol, 0.98 equiv.) was added. The mixture was stirred for 12 h, then partitioned between EtOAc and water and the aqueous re-extracted (EtOAc). The combined organics were washed (5% (w/v) LiCl_{(aq)}×2) and dried over MgSO₄, then concentrated under reduced pressure. The residue was redissolved and concentrated from DCM, MeCN and DCM again sequentially to fully remove residual DMF. Cyanomethyl ester 3 was isolated as an off-white solid (1.07 g, 85%). ¹H NMR (400 MHz, DMSO-d₆) δ 7.66 (d, J=8.8 Hz, 2H), 6.58 (d, J=8.8 Hz, 2H), 6.19 (s, br, 2H), 5.07 (s, 2H)¹³C NMR (126 MHz, DMSO-d₆) δ 164.6, 154.4, 131.7, 116.5, 113.4, 112.8, 48.8. HR-ESI-MS [M+H]⁺: calculated for C₉H₈N₂O₂, m/z 177.0659, found m/z 177.0658.

### Cyanomethyl 4-aminobenzoate (2)

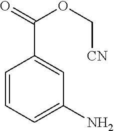 [SMILES: N#CCOC(=O)c1cccc(N)c1]

3-Aminobenzoic acid (200 mg, 1.46 mmol, 1.00 equiv.) was dissolved in DMF (1.0 mL) with potassium carbonate (404 mg, 2.92 mmol, 2.00 equiv.), then chloroacetonitrile (88 μL, 1.4 mmol, 0.95 equiv.) was added. The mixture was stirred for 20 h, then partitioned between EtOAc and water and the aqueous re-extracted (EtOAc). The combined organics were dried over MgSO₄, then concentrated under reduced pressure onto silica. Purified by chromatography (0-100% EtOAc in hexanes) to give cyanomethyl ester 2 as a white solid (158 mg, 65%). ¹H NMR (500 MHz, CDCl₃) δ 7.44-7.37 (m, 1H), 7.33 (app. t, J=2.1 Hz, 1H), 7.23 (app. t, J=7.9 Hz, 1H), 6.90 (ddd, J=8.0, 2.4, 1.0 Hz, 1H), 4.91 (s, 2H), 3.84 (s, 2H). ¹³C NMR (126 MHz, CDCl₃) δ 165.3, 146.9, 129.7, 128.8, 120.6, 120.0, 115.9, 114.7, 48.9. HR-ESI-MS [M+H]⁺: calculated for C₉H₈N₂O₂, m/z 177.0659, found m/z 177.0659.

### Cyanomethyl 2-aminobenzoate (1)

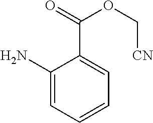 [SMILES: N#CCOC(=O)c1ccccc1N]

2-Aminobenzoic acid (200 mg, 1.46 mmol, 1.00 equiv.) was dissolved in DMF (1.0 mL) with potassium carbonate (404 mg, 2.92 mmol, 2.00 equiv.), then chloroacetonitrile (88 μL, 1.39 mmol, 0.952 equiv.) was added. The mixture was stirred for 21 h, then partitioned between EtOAc and water, Na₂SO_{4(aq.)} added, and the aqueous re-extracted (EtOAc). The combined organics were dried over MgSO₄, then concentrated under reduced pressure onto silica. Purified by chromatography (0-100% EtOAc in hexanes) to give cyanomethyl ester 1 as a white solid (158 mg, 83%). ¹H NMR (500 MHz, CDCl₃) δ 7.83 (dd, J=8.1, 1.6 Hz, 1H), 7.32 (ddd, J=8.5, 7.1, 1.6 Hz, 1H), 6.76-6.57 (m, 2H), 5.70 (s, 2H, NH₂), 4.90 (s, 2H). ¹³C NMR (126 MHz, CDCl₃) δ 166.3, 151.3, 135.4, 131.3, 116.9, 116.6, 114.9, 108.3, 48.4. HR-ESI-MS [M+H]⁺: calculated for C₉H₈N₂O₂, m/z 177.0659, found m/z 177.0660.

### 4-[(tert-butyldimethylsilyl)oxy]benzoic acid (25)

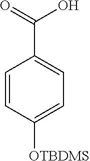 [SMILES: CC(C)(C)[Si](C)(C)Oc1ccc(C(=O)O)cc1]

4-hydroxybenzoic acid (2.00 g, 14.5 mmol, 1.00 equiv.) was dissolved in DMF (30 mL) with imidazole (2.96 g, 43.4 mmol, 3.00 equiv.), then TBDMSCl (6.55 g, 43.4 mmol, 3.00 equiv.) was added. The mixture was stirred for 44 h, then partitioned between Et₂O and water and the organic layer washed (water×1, brine×1), then the combined aqueous re-extracted (Et₂O). The combined organics were dried over MgSO₄, then concentrated under reduced pressure to give double silyl protected intermediate TBDMS as a clear oil (5.232 g) which was used without purification. The TBDMS ester (5.23 g, 14.3 mmol, 1.00 equiv.) was dissolved in THF (64 mL), then AcOH (48 mL) and water (16 mL) added. Heated to 50° C. for 5 h, then allowed to cool overnight. Concentrated under vacuum to give acid 25 as a white solid (3.20 g, 88%). ¹H NMR (400 MHz, CD₃OD) δ 7.93 (d, J=8.7 Hz, 2H), 6.90 (d, J=8.7 Hz, 2H), 1.01 (s, 9H), 0.25 (s, 6H). Procedure modified from prior literature procedure,⁷ spectra consistent with previous literature data (Candish, et al., Chem Sci, 8, 3618-3622 (2017)).

### Cyanomethyl 4-[(tert-butyldimethylsilyl)oxy]benzoate (26)

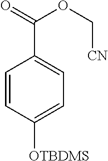 [SMILES: CC(C)(C)[Si](C)(C)Oc1ccc(C(=O)OCC#N)cc1]

Benzoic acid 25 (1.93 g, 7.66 mmol, 1.00 equiv.) was suspended in chloroacetonitrile (2.40 mL, 38.3 mmol, 5.00 equiv.) followed by addition of triethylamine (2.13 mL, 15.3, 2.00 equiv.), then the mixture stirred at rt for 16 h. The mixture was then partitioned between EtOAc and 0.5 M HCl_{(aq)} and the aqueous re-extracted (EtOAc) and the combined organics washed (brine, ×2), then dried over MgSO₄ and concentrated under reduced pressure. The residue was re-dissolved in DCM and loaded onto silica by evaporation, then purified by chromatography (0-15% EtOAc in hexanes) to give the cyanomethyl ester 26 as a colorless oil (1.302 g, 58%). ¹H NMR (400 MHz, CDCl₃) δ 7.96 (d, J=8.8 Hz, 2H), 6.89 (d, J=8.7 Hz, 2H), 4.93 (s, 2H), 0.99 (s, 9H), 0.24 (s, 6H). ¹³C NMR (126 MHz, CDCl₃) δ 164.7, 161.3, 132.3, 120.9, 120.3, 114.8, 48.7, 25.7, 18.4, −4.3. HR-ESI-MS [M−H−TBDMS]⁻: calculated for C₁₅H₂₁NO₃Si, m/z 176.0353, found m/z 176.0356.

### Cyanomethyl 4-hydroxybenzoate (16)

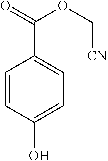 [SMILES: N#CCOC(=O)c1ccc(O)cc1]

TBDMS ether 26 (46 mg, 0.16 mmol, 1.0 equiv.) was dissolved in THF (0.50 mL), cooled to 0° C. and TBAF added to the stirring mixture (240 μL, 0.240 mmol, 1.50 equiv.). After 30 min the mixture was partitioned between EtOAc and 0.5 M HCl_{(aq)} then the aqueous re-extracted (a small amount of Na₂SO_{4(aq.)} was added to accelerate layer separation). The combined organics were washed (brine), then dried over MgSO₄ and concentrated under reduced pressure and loaded onto silica by evaporation. Purified by chromatography (0-100% EtOAc in hexanes) to give phenol 16 as a white solid (18 mg, 64%). ¹H NMR (400 MHz, Acetone-d₆) δ 9.34 (s, 1H), 7.94 (d, J=8.8 Hz, 2H), 6.97 (d, J=8.8 Hz, 2H), 5.13 (s, 2H). ¹³C NMR (126 MHz, Acetone-d₆) δ 165.4, 163.5, 133.0, 120.4, 116.4, 116.4, 49.6. HR-ESI-MS [M+H]⁺: calculated for C₉H₇NO₃, m/z 178.0499, found m/z 178.0500.

### 4-Chlorobenzyl mercaptan malonate (19)

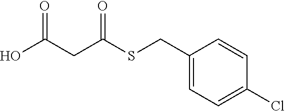 [SMILES: O=C(O)CC(=O)SCc1ccc(Cl)cc1]

Meldrum's acid (295 mg, 2.37 mmol, 1.00 equiv.) was suspended in toluene (5 mL) with 4-chlorobenzylmercaptan (320 uL, 2.62 mmol, 1.10 equiv.) and the solution was heated under reflux for 4 h. Allowed to cool, then partitioned between EtOAc and 0.5 M HCl_{(aq)}. The organic phase was then washed with 0.2 M NaHCO_{3(aq)}, followed by brine. The organic layer was dried over MgSO₄ and filtered. The resulting oil was re-suspended in 6 mL of 1:5 EtOAc:hexane and purified by chromatography (10-50% EtOAc in hexanes) to give 4-chlorobenzylmercaptan 19 as a white powder (49 mg, 10%). ¹H NMR (500 MHz, DMSO-d₆) δ 12.86 (s, 1H), 7.35 (m, 4H), 4.14 (s, 2H), 3.67 (s, 2H). ¹³C NMR (126 MHz, DMSO-d₆) δ 192.3, 167.3, 136.6, 131.8, 130.1, 128.5, 49.3, 31.8. HR-ESI-MS [M−H]⁻: calculated for C₁₀H₉ClO₃S, m/z 242.9888, found m/z 242.9881.

### N-Formylmethionyl 3,5-dintrobenzyl methyl ester (28)

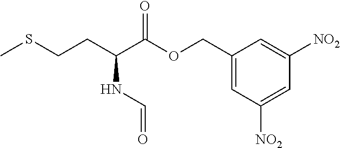 [SMILES: CSCC[C@H](NC=O)C(=O)OCc1cc([N+](=O)[O-])cc([N+](=O)[O-])c1]

N-formyl-L-methionine (280 mg, 1.32 mmol, 1.00 equiv.) and 3,5-dinitrobenzyl chloride (286 mg, 1.32 mmol, 1.00 equiv.) were suspended in 2.0 mL of DMF and triethylamine (372 μL, 2.65 mmol, 2.00 equiv.) was added. The reaction was stirred at rt for 18 h. The mixture was partitioned between EtOAc and 0.1 M HCl_{(aq)}. The organic layer was then washed sequentially with 0.2M NaHCO_{3(aq)}, water, and brine, then dried over MgSO₄ and filtered. The resulting oil was then re-suspended in 6 mL of 1:5 EtOAc:hexane and purified by chromatography (0-55% EtOAc in hexanes) to give the 3,5-dinitrobenzyl ester of 28 as a dark orange oil (143 mg, 40%). ¹H NMR (500 MHz, CDCl₃) δ 9.0 (s, 1H), 8.57 (d, J=2.0 Hz, 2H), 8.26 (s, 1H), 6.28 (d, J=7.5 Hz, 1H), 5.45-5.32 (m, 2H), 4.89 (td, J=7.5, 5.0 Hz, 1H), 2.57 (t, J=7.1 Hz, 2H), 2.26-2.09 (m, 2H), 2.10 (s, 3H). ¹³C NMR (126 MHz, CDCl₃) δ 171.2, 160.8, 148.8, 139.6, 128.2, 119.0, 65.1, 50.4, 31.2, 30.1, 15.8. HR-ESI-MS [M+H]⁺: calculated for C₁₃H₁₅N₃O₇S, m/z 358.0703, found m/z 358.0703.

### 3-((2-nitrobenzyl)oxy)-3-oxopropanoic acid (27)

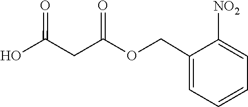 [SMILES: O=C(O)CC(=O)OCc1ccccc1[N+](=O)[O-]]

Adapting the procedure of Ryu et al. (Ryu, et al., Tet Lett, 44, 7499-7502 (2003)) 2,2-dimethyl-1,3-dioxane-4,6-dione (4.32 g, 30.0 mmol, 1.00 equiv.) and 2-nitrobenzyl alcohol (4.60 g, 30.0 mmol, 1.00 equiv.) were suspended in PhMe (30 mL). The reaction mixture was then heated to reflux for 4 h resulting in a homogenous solution. Upon completion, as judged by LCMS, the reaction was cooled to rt, diluted with DCM and saturated NaHCO_{3(aq)}. The aqueous layer was washed with DCM (×2), and the combined organics back-extracted with saturated NaHCO_{3(aq)}. The aqueous layer was cautiously acidified with 12 N HCl_{(aq)}, and the product extracted thrice with DCM. The combined organics were dried over Na₂SO₄, filtered and concentrated to afford the product as an off-white solid (6.38 g, 89%), which was used without further purification. ¹H NMR (400 MHz, DMSO-d₆) δ 12.91 (s, 1H), 8.13 (dd, J=8.2, 1.3 Hz, 1H), 7.79 (app td, J=7.5, 1.3 Hz, 1H), 7.73 (dd, J=7.8, 1.6 Hz, 1H), 7.62 (ddd, J=8.5, 7.3, 1.6 Hz, 1H), 5.50 (s, 2H), 3.52 (s, 2H)¹³C NMR (101 MHz, DMSO-d₆) δ 167.9, 166.6, 147.2, 134.2, 131.3, 129.3, 129.1, 124.9, 62.9, 41.4. HR-ESI-MS [M−H]⁻: calculated for C₁₀H₉NO₆, m/z 238.0357, found m/z 238.0352.

### Cyanomethyl (2-nitrobenzyl) malonate (22)

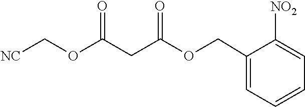 [SMILES: N#CCOC(=O)CC(=O)OCc1ccccc1[N+](=O)[O-]]

To a solution of 27 (598 mg, 2.50 mmol, 1.00 equiv.) in MeCN (2.5 mL) was added bromoacetonitrile (1.7 mL, 25 mmol, 10 equiv.), followed by addition of N-methylmorpholine (1.5 mL, 14 mmol, 5.5 equiv.) via syringe over 3 h. The reaction was allowed to stir for an additional 2 h, then diluted with Et₂O and quenched by addition of saturated NaHCO_{3(aq)}. The product was extracted thrice with Et₂O, and the combined organics washed with saturated aqueous NaCl, dried over MgSO₄, filtered and concentrated in vacuo. The resulting solid was then dissolved in 10 mL CHCl₃ and filtered through a cotton plug. The filtrate was concentrated to afford the desired product as a white solid (631 mg, 91%). ¹H NMR (400 MHz, CDCl₃) δ 8.10 (dd, J=8.2, 1.3 Hz, 1H), 7.68 (app td, J=7.6, 1.3 Hz, 1H), 7.60 (d, J=7.7 Hz, 1H), 7.56-7.46 (m, 1H), 5.59 (s, 2H), 4.81 (s, 2H), 3.59 (s, 2H)¹³C NMR (101 MHz, CDCl₃) δ 164.8, 164.8, 147.6, 134.10, 131.0, 129.4, 129.3, 125.3, 113.9, 64.4, 49.3, 40.6. HR-ESI-MS [M−H]⁻: calculated for C₁₂H₁₀N₂O₆, m/z 277.0466, found m/z 277.0470.

### 3-((3,5-dinitrobenzyl)oxy)-3-oxopropanoic acid (20)

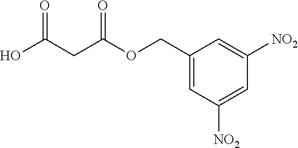 [SMILES: O=C(O)CC(=O)OCc1cc([N+](=O)[O-])cc([N+](=O)[O-])c1]

Adapting the procedure of Ryu et al., (Ryu, et al., Tet Lett, 44, 7499-7502 (2003)) 2,2-dimethyl-1,3-dioxane-4,6-dione (288 mg, 2.00 mmol, 1.00 equiv.) and 3,5-dinitrobenzyl alcohol (396 mg, 2.00 mmol, 1.00 equiv.) were suspended in PhMe (2.0 mL). The reaction mixture was then heated to reflux for 5 h resulting in a homogenous solution. Upon completion, as judged by LCMS, the reaction was cooled to rt, diluted with DCM and saturated NaHCO_{3(aq)}. The aqueous layer was washed with DCM (2×), and the combined organics back-extracted with saturated NaHCO_{3(aq)}. The aqueous layer was cautiously acidified with 12 N HCl_{(aq)}, and the product extracted with DCM (×3). The combined organics were dried over Na₂SO₄, filtered and concentrated to afford the product as a pale yellow solid (300 mg, 53%) which was used without further purification. ¹H NMR (400 MHz, DMSO-d₆) δ 12.95 (s, 1H), 8.79 (t, J=2.2 Hz, 1H), 8.69 (d, J=2.1 Hz, 2H), 5.43 (s, 2H), 3.55 (s, 2H)¹³C NMR (101 MHz, DMSO-d₆) δ 168.1, 166.7, 148.1, 140.5, 127.9, 118.1, 64.0, 41.4. HR-ESI-MS [M−H]⁻: calculated for C₁₀H₈N₂O₈, m/z 283.0208, found m/z 283.0203.

### 3,5-Dinitrobenzyl methyl malonate (23)

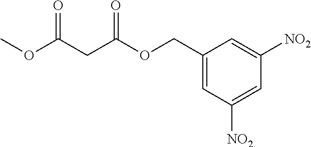 [SMILES: COC(=O)CC(=O)OCc1cc([N+](=O)[O-])cc([N+](=O)[O-])c1]

To a solution of 20 (71 mg, 0.250 mmol, 1.00 equiv.) in MeOH/PhMe (1:3 v/v, 2.5 mL) was added TMSCHN₂ (2.0 M, 0.3 mL, 0.6 mmol) until a yellow color persisted, during which the evolution of N_{2(g)} was observed. The reaction was allowed to stir vigorously for an additional 30 min. Next, SiO₂ was added and the mixture stirred for 15 minutes to quench excess TMSCHN₂. The SiO₂ was filtered off, and the filtrate concentrated under reduced pressure. The crude material was purified by chromatography (SiO₂, 5-50% EtOAc/hexanes) to obtain the desired malonate ester 23 as a pale yellow oil (66 mg, 89%). ¹H NMR (400 MHz, CDCl₃) δ 9.00 (t, J=2.1 Hz, 1H), 8.56 (d, J 1.7 Hz, 2H), 5.39 (d, J=0.8 Hz, 2H), 3.79 (s, 3H), 3.52 (s, 2H). ¹³C NMR (101 MHz, CDCl₃) δ 166.5, 165.9, 148.8, 140.1, 127.7, 118.7, 64.6, 53.0, 41.2. HR-ESI-MS [M+H]⁺: calculated for C₁₁H₁₀N₂O₈, m/z 299.0510, found m/z 299.0512.

### 5-benzyl-2,2-dimethyl-1,3-dioxane-4,6-dione (29) (Engl, et al., Helv Chim Acta, 100, e1700196 (2017))

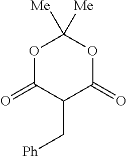 [SMILES: CC1(C)OC(=O)C(Cc2ccccc2)C(=O)O1]

According to the procedure of Engl et al. (McMurry, et al. Proc Natl Acad Sci USA, 114, 11920-11925 (2017)) to a suspension of benzyl malonic acid (582 mg, 3.00 mmol, 1.00 equiv.) in acetic anhydride (6.0 M, 0.5 mL). H₂SO_{4(aq)} (18 M, 15 μL) was added and the reaction mixture was cooled to 0° C., followed by dropwise addition of acetone (0.3 mL). The reaction mixture was allowed to warm to rt and stirred overnight. The product was then precipitated by addition of ice. The solid was collected by vacuum filtration and washed with cold water to afford the desired product as an off-white solid (407 mg, 57%). ¹H NMR (400 MHz, CDCl₃) δ 7.38-7.17 (m, 5H), 3.76 (t, J=4.9 Hz, 1H), 3.49 (d, J=5.0 Hz, 2H), 1.73 (s, 3H), 1.49 (s, 3H)¹³C NMR (101 MHz, CDCl₃) δ 165.5, 137.4, 129.9, 128.8, 127.4, 105.4, 48.3, 32.3, 28.6, 27.4 HR-ESI-MS [M−H]⁻: calculated for C₁₃H₁₄O₄, m/z 233.0819, found m/z 233.0815.

### 2-benzyl-3-((3,5-dinitrobenzyl)oxy)-3-oxopropanoic acid (21)

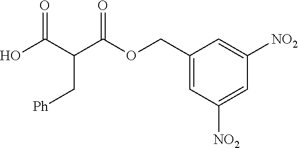 [SMILES: O=C(O)C(Cc1ccccc1)C(=O)OCc1cc([N+](=O)[O-])cc([N+](=O)[O-])c1]

Crude ester 29 (234 mg, 1.00 mmol, 1.00 equiv.) and 3,5-nitrobenzyl alcohol (198 mg, 1.00 mmol, 1.00 equiv.) were suspended in PhMe (1.0 mL). The reaction mixture was then heated to 110° C. for 3 h resulting in a homogenous solution. Upon completion, as judged by LCMS, the reaction was cooled to rt and concentrated to dryness under reduced pressure. The product was triturated with cold Et₂O/Hexanes (1:2 v/v, 30 mL) and collected by vacuum filtration to afford the title compound. 21 as a tan solid (252 mg, 67%). ¹H NMR (400 MHz, DMSO-d₆) δ 13.13 (s, 1H), 8.78 (t, J=2.1 Hz, 1H), 8.55 (d, J=2.1 Hz, 2H), 7.29-7.04 (m, 5H), 5.43-5.27 (m, 2H), 3.92 (dd, J=8.9, 7.0 Hz, 1H), 3.13 (dd, J=14.0, 7.1 Hz, 1H), 3.06 (dd, J=14.0, 9.0 Hz, 1H)¹³C NMR (101 MHz, DMSO-d₆) δ 169.6, 168.7, 148.0, 140.2, 137.8, 128.6, 128.2, 128.0, 126.4, 118.1, 64.0, 52.9, 34.1. HR-ESI-MS [M+H]⁺: calculated for C₁₇H₁₄N₂O₈, m/z 375.0823, found m/z 375.0821.

### Example 2: tRNA can be Acylated with Aminobenzoic Acids

### Materials and Methods

Commercial Materials

DNase-free water, magnesium chloride solution, sodium acetate solution (pH 5.2), 20,000× ethidium bromide, and ethanol were purchased from AmericanBio (Canton, Mass.). Flexizyme RNA (eFx and dFx) along with microhelix RNA were purchased from Integrated DNA Technologies (Coralville, Iowa). DNA oligonucleotides were purchased from the Keck Biotechnology Resource Labs (New Haven, Conn.). HiScribe in vitro transcription kit and PureExpress (ΔtRNA, Δaa) were purchased from New England Biolabs (Ipswich, Mass.). RNAse-free DNAse I, dimethylsulfoxide (DMSO), HEPES, phenol, chloroform, methanol, trichloroacetic acid (TCA), ethyl acetate, dichloromethane (DCM), magnesium acetate, sodium chloride, were purchased from Sigma-Aldrich (St. Louis, Mo.).

tRNA Synthesis, Purification, and Folding

DNA templates used for transcribing E. coli tRNA^{fMet}_{CAU}, tRNA^{Val}_{UAC}, and tRNA^{Val}_{CUA} were prepared using polymerase chain reactions (PCR) by annealing and extending oligonucleotides MetT-F and MetT-R, ValT-F and ValT-R, ValTam-F and ValT-R, respectively (Table 1).

TABLE 1
(A) DNA oligonucleotides used for this study.
(B) RNA oligonucleotides used for this study
A. DNA oligonucleotide sequences

| me | Sequence |
|---|---|
| lT-F | AATTCCTGCAGTAATACGACTCACTATAGGGTG |
| | ATTAGCTCAGCTGGGAGAGCACCTCCCTTACAA |
| | GGAGGGGGTCGGC (SEQ ID NO: 4) |
| ValTam-F | AATTCCTGCAGTAATACGACTCACTATAGGGTG |
| | ATTAGCTCAGCTGGGAGAGCACCTCCCTCTAAA |
| | GGAGGGGGTCGGC (SEQ ID NO: 5) |
| ValT-R* | TmGGTGGGTGATGACGGGATCGAACCGCCGACC |
| | CCCTCCTT (SEQ ID NO: 6) |
| MetT-F | AATTCCTGCAGTAATACGACTCACTATACGCGG |
| | GGTGGAGCAGCCTGGTAGCTCGTCGGGCTCATA |
| | (SEQ ID NO: 7) |
| MetT-R* | TmGGTTGCGGGGGCCGGATTTGAACCGACGACC |
| | TTCGGGTTATGAGCCCGACGAGCTA |
| | (SEQ ID NO: 8) |
| MVFflag-1 | TAATACGACTCACTATAGGGTTAACTTTAACAA |
| | GGAGAAAAACATGGTATTTGACTACAAGG |
| | (SEQ ID NO: 9) |
| MVFflag-2 | CGAAGCTTACTTGTCGTCGTCGTCCTTGTAGTC |
| | AAATACCATGTTTTTCTCCTTGTTAAAG |
| | (SEQ ID NO: 10) |
| MVFflag-3 | GCGAATTAATACGACTCACTATAGGGTTAACTT |
| | TAACA (SEQ ID NO: 11) |
| MVFflag-4 | AAACCCCTCCGTTTAGAGAGGGGTTATGCTAGT |
| | TACTTGTCGTCGTCGTCCTTG |
| | (SEQ ID NO: 12) |

B. RNA oligonucleotide sequences

| Name | Sequence |
|---|---|
| dFx | GGAUCGAAAGAUUUCCGCAUCCCCGAAAGGGUA |
| | CAUGGCGUUAGGU (SEQ ID NO: 1) |
| eFx | GGAUCGAAAGAUUUCCGCGGCCCCGAAAGGGGA |
| | UUAGCGUUAGGU (SEQ ID NO: 2) |
| Microhelix | GGCUCUGUUCGCAGAGCCGCCA |
| tRNA | (SEQ ID NO: 13) |

*TmG represents ²O-methyl-deoxymethylguanosine

Each template was then extracted with a 1:1 (v/v) phenol/chloroform solutions and precipitated in 3 volumes of 95% ethanol. T7 HiScribe RNA synthesis kit (New England Biolabs (NEB)) was used to transcribe each tRNA in 200 μl reactions containing 10 μg of DNA template. Transcription reactions were incubated at 37° C. for 6 hours, then 100 U of RNAse-free DNAse I (Sigma-Aldrich) was added to digest template DNA for 2 additional hours. Sodium acetate, pH 5.2 was added to 200 mM and RNA was extracted with acid phenol, twice with chloroform, then precipitated in 3 volumes of 95% (v/v) ethanol. RNA pellets were washed twice with 70% (v/v) ethanol and resuspended in RNAse-free water. Each sample was purified using RNAse-free Micro Bio-Spin P-30 Tris columns (Bio-Rad) following the manufacturer's protocol. tRNAs were folded by boiling for 5 minutes at 95° C. in a heat block then slowly cooling over 2 hours to room temperature. Magnesium chloride was added to 10 mM when tRNA samples cooled to 65° C.

Acylation of Microhelix tRNA Using Flexizymes

Acylation of microhelix tRNA protocols were modified from a previously reported methods (Goto, et al., Methods Mol Biol, 848, 465-78 (2012), Fujino, J Am Chem Soc, 138, 1962-9 (2016)) 1 μL of 250 μM Flexizyme (dFx or eFx) was added to 1 μL of 500 mM HEPES (pH 7.5, Sigma-Aldrich) or 500 mM Bicine (pH 9, Hampton Research, Aliso Viejo, Calif.) and 1 μL of 250 μM microhelix tRNA. The sample was then incubated at 95° C. for 2 min and allowed to come to room temperature in 5 min. 6 uL of 1 M Magnesium chloride was then added, followed by 1 uL of DMSO solution of CME, DBE, or CBT variants (50 mM). The reaction was then incubated at 4° C. for 48 h. Acylation was analyzed by gel-shift similarly to as described in previous work methods (Goto, et al., Methods Mol Biol, 848, 465-78 (2012)). Specifically, equal volume of acid-PAGE buffer (150 mM sodium acetate (pH 5.2), 10 mM EDTA (AmericanBio), 950 μL formamide (AmericanBio), 0.2 mg bromophenol blue (Sigma-Aldrich)) was added to the crude reactions and 2 uL of each sample was run on an acid-urea PAGE-gel (20% acrylamide, 36% Urea (w/v) (Sigma-Aldrich), 50 mM Sodium Acetate (pH 5.2) (AmericanBio), 0.1% Ammonium Persulfate (w/v) (Sigma-Aldrich), 0.08% TEMED (v/v) (Sigma-Aldrich). The gel was run at room temperature at 120 V over the course of 3.5-4 h with 50 mM Sodium Acetate (pH 5.2) as the running buffer. To image the gel, it was stained by Ethidium Bromide (AmericanBio) in 50 mL TBE (12.8 g/L TRIS (Sigma-Aldrich), 5.5 g/L Boric Acid (Sigma-Aldrich), 10 mM EDTA (AmericanBio), pH 8.0) for 1-2 min. The gel was destained in 50 mL TBE for 1 min and imaged on a ChemiDoc (Bio-Rad). UV densitometry was carried out using ImageJ (NIH, Bethesda, Md.). The HEPES (pH 7.5) buffer system was used with (Phe-CME, β-Phe-CME, fMet-DBE, 8, and 19-23) and the Bicine (pH 9) buffer system was used with (1-6 and 9-18).

Characterization of Acylated tRNA Using RNAse A Digestion and LC-MS

Characterization of acylated tRNA was achieved by digestion of tRNA or microhelix tRNA using RNAse A as described previously (McMurry, et al., Proc Natl Acad Sci USA, 114, 11920-11925 (2017)). 1 μL of the microhelix tRNA acylation reactions described above was removed prior to PAGE analysis and quenched with 1.1 μL RNAse A (1.5 U/μL, 200 mM Sodium Acetate, pH 5.2) (Sigma-Millipore). After 5 min incubation at r.t, RNAse A was precipitated by the addition of 50% trichloroacetic acid (TCA, Sigma-Aldrich)) to a final volume (v/v) of 5%. After 5 min at r.t, the sample was diluted to 20 μL and frozen by incubation at −80° C. for 5 min. Insoluble material and debris were removed by centrifugation at 21,300×g for 10 min at 4° C. For characterization, the samples were analyzed on a C₁₈ RRHD column (1.8 μm, 2.1×50 mm, r.t, Agilent) using a linear gradient from 4 to 40% acetonitrile over 1.25 min followed by 40% to 100% for 0.4 min with 0.1% formic acid as the aqueous mobile phase after an initial hold at 4% acetonitrile for 1.35 min (0.7 mL/min) using a 1290 Infinity II UHPLC (G7120AR, Agilent). Acylation was confirmed by correct identification of the exact mass of the 2′ and 3′ acyl-adenosine using LC-HRMS with an Agilent 6530 QTOF AJS-ESI (G6230BAR). The following parameters were used: Fragmentor voltage 175 V, Gas temperature 300° C., Gas flow 12 L/min, Sheath gas temperature 350° C., Sheath gas flow 12 L/min, Nebulizer pressure 35 psi, skimmer voltage 65 V, Vcap 3500 V, 3 spectra/s.

Formation of Anthraninoyl-tRNA Using Isatoic Anhydride

This reaction was modified from a previously established protocol (Nawrot, et al., Nucleosides Nucleotides, 17, 815-29 (2017)) tRNA^{Val}_{CUA} or tRNA^{fMet}_{CAU}, (25-200 uM) were incubated with 2-5 mM NaOH in 90% acetonitrile with 8-80 mM isatoic anhydride (Sigma-Aldrich) for 3 h at 37° C. The total reaction volume ranged from 10-200 μL. After 3 h, the sample was diluted with 800 uL of nuclease free water and flash frozen in a dry-ice/acetone bath. The sample was then lyophilized to dryness and resuspended in 20-200 uL 300 mM Sodium Acetate (AmericanBio). The insoluble material was removed by centrifugation at 21,300×g for 10 min at 4° C. The tRNA concentration was determined by Nanodrop. When used for in vitro translation reactions, this material was used directly. To analyze acylation of the 3′-hydroxyl of adenosine, RNAse A (1.5 U/μL, 200 mM Sodium Acetate, pH 5.2) was added in 1.1 volumes. After 5 min incubation at r.t, RNAse A was precipitated by the addition of 50% trichloroacetic acid (TCA, Sigma-Aldrich) to a final volume (v/v) of 5%. After 5 min at r.t, the sample was diluted 10-fold and frozen by incubation at −80° C. for 5 min. Insoluble material and debris were removed by centrifugation at 21,300×g for 10 min at 4° C. For characterization, the samples were analyzed on a C18 RRHD column (1.8 μm, 2.1×50 mm, r.t, Agilent) using a linear gradient from 4 to 40% acetonitrile over 1.25 min followed by 40% to 100% for 0.4 min, with 0.1% formic acid as the aqueous mobile phase after an initial hold at 4% acetonitrile for 1.35 min (0.7 m/min) using a 1290 Infinity II UHPLC (G7120AR, Agilent). Acylation was confirmed by correct identification of the exact mass of the 2′ and 3′ acyl-adenosine using LC-HRMS with an Agilent 6530 QTOF AJS-ESI (G6230BAR). The following parameters were used: Fragmentor voltage 175 V, Gas temperature 300° C., Gas flow 12 L/min, Sheath gas temperature 350° C., Sheath gas flow 12 L/min, Nebulizer pressure 35 psi, skimmer voltage 65 V, Vcap 3500 V, 3 spectra/s.

Analysis of Intact tRNA by Liquid Chromatography

The samples were analyzed on a C18 AdvanceBio Oligonucleotide column (2.7 μm, 2.1×50 mm, 50° C., Agilent) using a linear gradient from 0 to 30% methanol over 10 min with 5 mM ammonium acetate (not pH adjusted) as the aqueous mobile phase (0.2 mL/min) using a 1290 Infinity II UHPLC (G7120AR, Agilent). The tRNAs were analyzed for UV absorbance at 260 nm using a UV detector (1290 Infinity II DA detector with 60 mm flow cell ((G7117BR), Agilent).

Results

One interesting family of foldamer-like molecules are aramids, oligomers of substituted aminobenzoic acids (Garcia et al., Angew. Prog. Polym. Sci, 35, 623-686 (2010)). Aramids possess remarkably varied properties. Kevlar, a polymer of 1,4-phenylenediamine and terephthaloyl chloride, is a strong and heat-resistant fiber (Tanner et al., Chem. Int. Ed Engl., 28, 649-654 (1989)), whereas cystobactamids are DNA gyrase inhibitors active against Gram-negative bacteria (Baumann et al., Angew. Chem. Int. Ed Engl., 53, 14605-14609 (2014)). Many additional aramid foldamers with remarkable properties have been reported (Saraogi et al., Angew Chem Int Ed Engl, 47, 9691-4 (2008), Meisel et al., Org Lett, 20, 3879-3882 (2018), Saha et al., Angew Chem Int Ed Engl, 57, 13542-13546 (2018)).

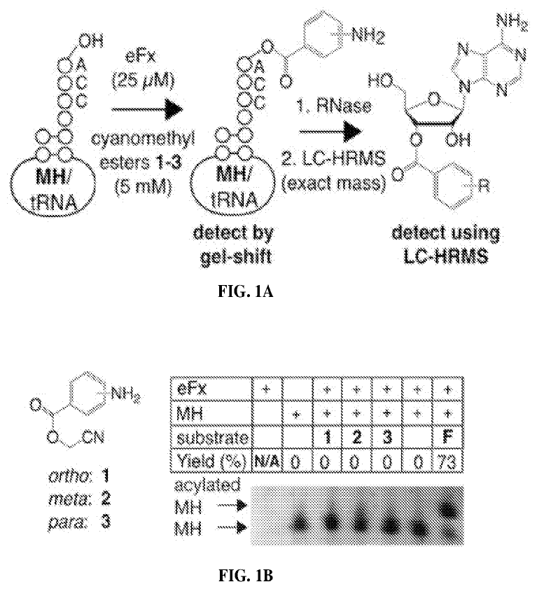

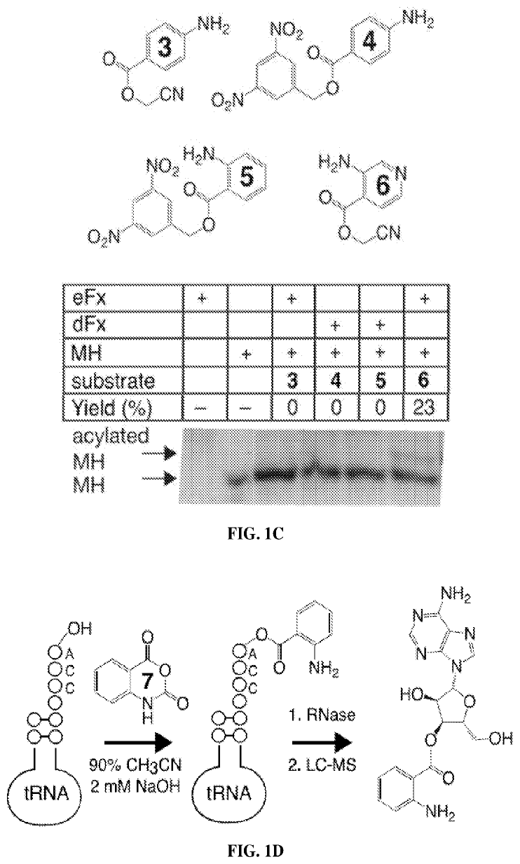

As the first step towards the ribosomal synthesis of aramid-like peptides, an established microhelix (MH) gel-shift assay (Goto et al., Protoc exch (2011)) and high-resolution mass spectrometry (FIG. 1A) were used to evaluate whether the cyanomethyl esters of unsubstituted aminobenzoic acids were substrates for the Flexizyme ribozyme eFx (Murakami et al., Chem. Biol., 10, 655-662 (2003)). Incubation of cyanomethyl esters 1-3 (5 mM) with 25 μM microhelix MH and 25 μM eFx in bicine buffer at pH 9 for 48 h showed little or no evidence of MH acylation when the reaction products were evaluated on an acid-urea PAGE gel (FIG. 1B). A low level of MH acylation by the m- and o-analogs 1 and 2 (and a trace with 3) could be observed using a highly sensitive RNAse A/LC-HRMS assay (McMurry & Chang, Proc. Natl. Acad. Sci. U.S.A, 114, 11920-11925 (2017)) that detects the acylated adenine nucleoside.

The extent of tRNA acylation was also investigated using the alternative ribozyme dFx (Murakami et al., Nat. Methods, 3, 357-359 (2006)) and the 1,3-dinitrobenzyl esters of p- and o-aminobenzoic acid (4 and 5, respectively). These substrates also failed to yield the expected MH products when incubated with dFx under standard conditions and analyzed using acid-urea gels or RNAse A/LC-HRMS (FIG. 1C), perhaps due to insolubility (Fujino et al., J. Am. Chem. Soc., 138, 1962-1969 (2016)). Even the more soluble cyanomethyl ester of ortho-aminonicotinic acid analog 6 reacted poorly in the presence of eFx (FIG. 1C).

Following the inability to efficiently acylate MH or tRNA with simple aminobenzoic acids in high yields using eFx or dFx, chemical acylation methods for the preparation of these materials were tested. Isatoic anhydride can acylate the terminal 2′- or 3′—OH group of an unprotected tRNA and the resulting anthraniloyl-tRNA (o-AN-tRNA) retains the ability to associate productively with EF-Tu-GTP (Nawrot & Sprinzl, Nucleosides and Nucleotides, 17, 815-829 (1998)). Next, E. coli tRNA^{Val} (ValT) or initiator tRNA (fMetT) was incubated with 8-80 mM isatoic anhydride in 90% CH₃CN containing 2-5 mM NaOH for 3 h at 37° C., digested the products with RNase A, and used LC-HRMS to detect the formation of nucleoside 7 (m/z=387.1411); this product will be observed only if reaction occurs at the tRNA 3′-end (FIG. 1D). A peak corresponding to this mass was observed only in reactions containing tRNA, isatoic anhydride, and base; in the absence of base, the acylation efficiency dropped by 1-2 orders of magnitude. Mindful of the fact that isatoic anhydride reagents can also modify RNA on the 2′—OH group of internal ribose residues in SHAPE reactions (Mortimer & Weeks, J. Am. Chem. Soc., 129, 4144-4145 (2007)), the reaction was also evaluated using UPLC, which showed evidence of multiple reaction products, whereas eFx-promoted reactions did not.

### Example 3: tRNA Charged with Aminobenzoic Acids is a Substrate for Translation

### Materials and Methods

Formation of Acyl-tRNAs Used for Protein Synthesis

Aminoacylation of tRNA^{fMet}_{CAU} and tRNA^{Val}_{UAC} was carried out using the same protocol as the microhelix tRNA with the only change being the use of tRNA^{fMet}_{CAU} or tRNA^{Val}_{UAC} instead of microhelix tRNA and the reaction volume (50-100 μL). Reactions where incubated at 4° C. from 48-72 h (as needed based on the microhelix tRNA acylation results). The reaction was quenched by addition of Sodium Acetate (pH 5.2) to a final volume of 300 mM and ethanol was added to a final volume of 70% (v/v). The sample was then incubated at −80° C. for 1 h and the RNA was pelleted by centrifugation at 21,300×g for 30 min at 4° C. The supernatant was removed and the pellet was washed with 500 μL of 70% (v/v) ethanol (stored at −20° C.). The sample was then centrifuged at 21,300×g for 7 min at 4° C. and the supernatant was removed. The pellet was air-dried for 2-5 min either at r.t or on ice. When used immediately, the pellet was resuspended in 1 mM Sodium Acetate (pH 5.2). If used at a later date, the pellet was stored dry at −80° C. and resuspended in 1 mM Sodium Acetate (pH 5.2) before use. To confirm acylation, a small fraction of the sample was subjected to RNAse A digestion and LC-MS analysis, as described above.

Synthesis and Purification of Translation Template

Templates for expression of MVFDYKDDDDK (SEQ ID NO:14) were generated by annealing and extending the oligonucleotides MVFflag-1 with MVFflag-2 (Table 1). The product from each reaction was then further amplified by PCR using primers MFflag-3 with MFflag-4 (Table 1). The dsDNA template was then extracted with a 1:1 (v/v) phenol/chloroform solutions and precipitated in 3 volumes of 95% (v/v) ethanol.

Ribosomal Synthesis of Short Peptides Initiated with Unnatural Carboxylic Acid Esters

In vitro transcription/translation of short peptides containing a FLAG tag fMet-Val-Phe-Asp-Tyr-Lys-Asp-Asp-Asp-Asp-Lys (fMVF-Flag) (SEQ ID NO:15) was carried out using the PureExpress (ΔtRNA, Δaa (E6840S)) kit by New England Biolabs with the following modifications. To generate the fMVF-Flag WT peptide the following reactions were executed (25 μL): Solution A ((ΔtRNA, Δaa) (5 μL), 33 mM Methionine (0.25 μL), 33 mM Valine (0.25 μL), solution containing 33 mM Tyrosine, 33 mM Phenylalanine, 33 mM Lysine (0.25 μL), 7 mM Asparatic acid (pH 7, 1 μL), tRNA solution (2.5 μL), Solution B (7.5 μL), 500-1000 ng dsDNA template (0.25-2 μL), and (water to 25 μL). When using precharged tRNA^{fMet}_{CAU} or tRNA^{Val}_{UAC}, either Valine or Methionine where omitted from the reaction mixture. The reactions were then incubated for 6 h at 37° C. Reactions incubated for 12-16 h did not show increased yields compared to reactions incubated for 6 h. The reactions were quenched by placing the reaction on ice and adding of 25 μL of dilution buffer (10 mM Magnesium Acetate (Sigma-Aldrich) and 100 mM Sodium Chloride (Sigma Aldrich)). To remove the proteins and majority of nucleic acid macromolecules, 5 μL of Ni-NTA (Qiagen, Hilden, Germany) slurry was added and the solution was incubated with light agitation at 4° C. for 1 h. The Ni-NTA resin was removed by centrifugation at 21,300×g for 10 min at 4° C. The supernatant was then frozen at −80° C. for 5 min and centrifuged once more at 21,300×g for 10 min at 4° C. The supernatant was analyzed on a AdvanceBio Peptide Map (2.7 μm, 2.1×100 mm, r.t, Agilent) column using a linear gradient from 0 to 55% acetonitrile and 0.1% over 6.5 min with 0.1% formic acid as the aqueous mobile phase after an initial hold at 95% 0.1% formic acid for 0.5 min (0.7 mL/min) using an 1290 Infinity II UHPLC (G7120AR, Agilent). Peptides were identified using LC-HRMS with an Agilent 6530 QTOF AJS-ESI (G6230BAR). The following parameters were used: Fragmentor voltage 200 V, Gas temperature 300° C., Gas flow 12 L/min, Sheath gas temperature 350° C., Sheath gas flow 11 L/min, Nebulizer pressure 35 psi, skimmer voltage 75 V, Vcap 3500 V, 1 spectra/s. For initial rate studies, aliquots of 4.5 μL where removed at each time point, immediately frozen in at −80° C., and stored at −80° C. until further analysis and purification. Peptides synthesized in the initial rate studies were purified and analyzed as described above.

### Results

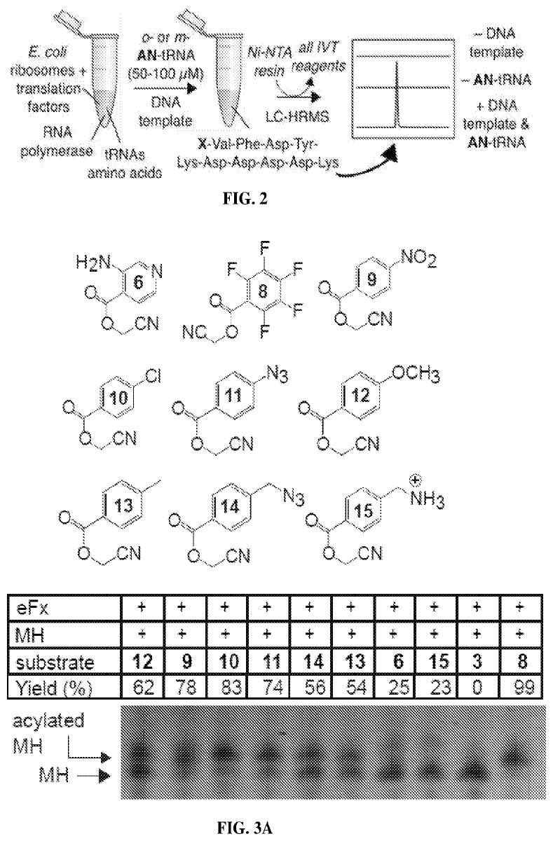

A commercial in vitro translation kit (PURExpress® Δ (aa, tRNA) was used to evaluate if an initiator tRNA (fMetT) acylated with o-(prepared using isatoic anhydride) or m-aminobenzoic acid (prepared using eFx) would be accommodated by the P-site of wild type E. coli ribosomes and initiate translation. The kit was supplemented with the requisite amino acids and tRNAs, pre-charged initiator tRNA (o- or m-AN-tRNA) (50-100 μM), and a duplex DNA template (0.5-1 μg) encoding the FLAG-containing polypeptide MVFDYKDDDDK (MVF-FLAG) (SEQ ID NO:14). After a 6 h incubation, the reaction mixture was treated with Ni-NTA resin to remove all PURExpress® A components (which are His₆-tagged) and the remaining material was analyzed by LC-HRMS (FIG. 2). If the o- or m-AN-tRNA initiates translation in place of an initiator tRNA charged with formyl methionine (fMet), then a polypeptide product containing the sequence AN-VFDYKDDDDK (AN-VF-FLAG) (SEQ ID NO:16) should be observed.

Parallel experiments were performed using the elongator tRNA ValT acylated (using eFx) with f-Phe (Fujino et al., J. Am. Chem. Soc., 138, 1962-1969 (2016)). Clear evidence for formation of a peptide carrying an aminobenzoic acid monomer was observed only in the presence of both DNA template and o-AN-tRNA. The identity of this product was further confirmed by isotope labeling experiments that showed the expected mass shift when the reaction was supplemented with ¹³C-labeled Phe. No AN-VF-FLAG polypeptide was detected in the presence of DNA template and m-AN-tRNA.

See also Table 2 below.

### Example 4: tRNA can be Charged with Substituted Benzoic Acid Cyanomethyl Esters, and Serve as a Substrate for Translation

Aminobenzoate esters hydrolyze exceptionally slowly (Drossman et al., Chemosphere, 17, 1509-1530 (1988)), indicating that the electron-rich aromatic ring contributes to the low reactivity of 1-3. In addition, the structure of the ethyl ester of L-phenylalanine bound to Fx (as an Fx-tRNA fusion) (Xiao et al., Nature, 454, 358-361 (2008)) shows pi-stacking between Fx base guanine 24 and the L-phenylalanine aromatic ring; this stacking would be less favorable with an electron-rich arene (Hansch et al., Chem. Rev., 91, 165-195 (1991)).

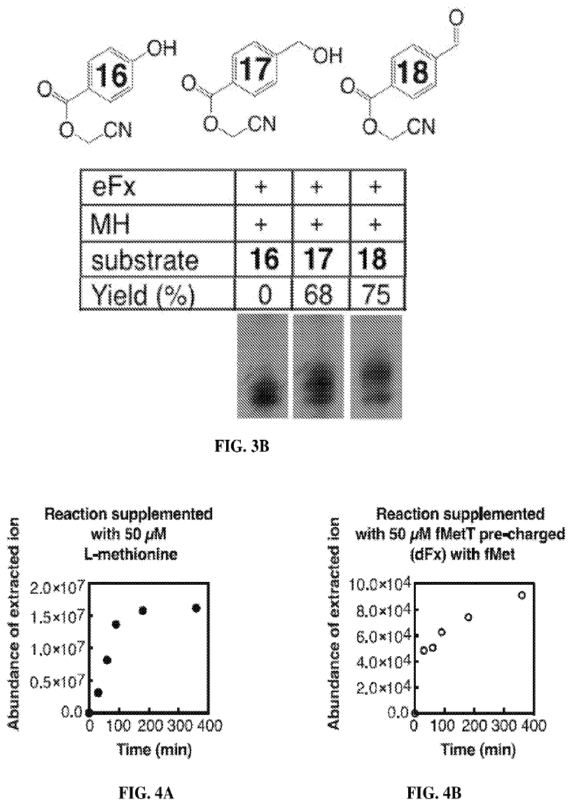

To investigate whether reactivity in eFx-promoted reactions was correlated with arene electron density, a diverse set of substituted benzoic acid cyanomethyl esters were prepared and evaluated for the extent to which eFx reactivity correlated with the sign and magnitude of the relevant sigma factor, which measures the inductive effect of the aromatic substituent (FIG. 3A) (Hansch et al., Chem. Rev., 91, 165-195 (1991)). Benzoic acid cyanomethyl esters possessing strong electron-withdrawing substituents, such as penta-fluoro 8, p-nitro 9 (σ=+0.78), or p-Cl 10 (σ=+0.23), were excellent eFx substrates in model MH reactions, with yields between 99 and 78% (FIG. 3A). But other factors are clearly important: a benzoic acid cyanomethyl ester possessing a weak electron-withdrawing substituent, such as p-azido 11 (σ=+0.08) was also an excellent substrate (yield of acylated MH=74%), as were analogs possessing both strong and weak electron-donating substituents, such as p-methoxy 12 (σ=−0.27; yield of acylated MH=62%) and p-methyl 13 (σ=−0.17; yield of acylated MH=54%). Notably, the poorest yields were observed in eFx-promoted reactions of substrates 6 (yield of acylated MH=25%) and 15 (yield of acylated MH=23%), all of which contain one or more acidic protons/hydrogen bond donors, just like amino benzoic acids 1, 2, and 3. These results imply that the presence of hydrogen-bond donors in certain positions can also contribute to the poor reactivity of amino benzoic acids 1-3. Consistent with this notion, p-hydroxybenzoic acid 16 (pKa=8.3 (methyl ester) was a poor substrate, whereas alcohol 17 (pKa=15 (benzyl alcohol)) and aldehyde 18 reacted well (FIG. 3B). It is possible that certain hydrogen bond donors alter the position of the aromatic ring in the eFx active site or coordinate and inactivate functional molecules involved in catalysis.

With a new set of aramid substrates in hand, the PURExpress® Δ (aa, tRNA) in vitro translation kit was used to evaluate if initiator tRNAs acylated with diverse benzoic acids could be accommodated in the ribosomal P-site and initiate translation of an AR-VF-FLAG polypeptide carrying an aramid monomer (AR) at the N-terminus. Every benzoic acid cyanomethyl ester that acylated the microhelix MH with a yield >50% in an eFx-promoted reaction (FIG. 3A) was used to acylate fMetT, and translation reactions were performed and analyzed as described above. With one exception, every single AR-fMetT initiated translation of an AR-VF-FLAG peptide whose mass corresponded to incorporation of the prescribed substituted benzoic acid. The singular exception was p-azidobenzoic acid 11; in this case the mass of the isolated polypeptide was consistent with in situ reduction of the azide to an amine. These results demonstrate that diverse aramid-like monomers can be accommodated directly within the ribosomal P-site and act as acceptors for a natural α-amino acid in the A-site. They show further that use of p-azidobenzoic acid 11 effectively circumvents the poor reactivity of p-aminobenzoic acid 3 to generate a polypeptide with a p-aminobenzoic acid monomer at the N-terminus. The observation that wild type E. coli ribosomes can initiate translation using tRNAs acylated with diverse aramid-like monomers significantly expands the scope of in vitro translation reactions beyond that of Kawakami (Kawakami et al., ACS Chem. Biol., 11, 1569-1577 (2016)) and lays the groundwork for the biosynthesis of genetically encoded, sequence-defined polyaramid oligomers.

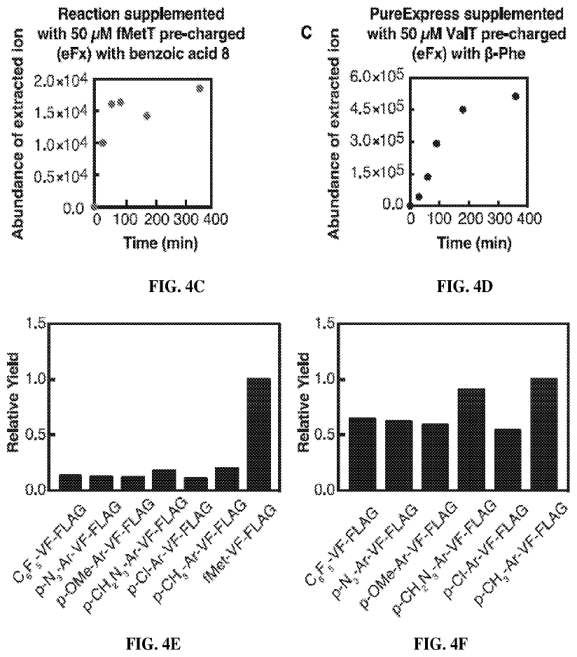

Next, the relative efficiency of PURExpress® reactions initiated with differentially acylated fMetT derivatives were evaluated. To begin, the yield of fMet-VF-FLAG (approximated as the extracted ion abundance) was monitored as a function of time in PURExpress® A reactions supplemented with either 50 μM pre-charged fMetT-fMet (charged using the dFx substrate fMet-DBE) or 50 μM of L-methionine. Both reactions reached saturation within 100 minutes, but the yield of fMet-VF-FLAG in reactions supplemented with pre-charged fMetT-fMet was 1.5% of that obtained in reactions supplemented with L-methionine (FIG. 4A-4B). Next, the extracted ion abundance (after 30-90 min) of the AR-VF-FLAG peptide initiated with fMetT pre-charged with benzoic acid ester 8 was compared. The yield of this AR-VF-FLAG polypeptide (p-C6H5-VF-FLAG) was 25-30% of the yield of fMet-VF-FLAG (generated in reactions supplemented with pre-charged fMetT-fMet) (FIG. 4C) and within the range observed when translation was initiated with fMetT pre-charged with natural amino acids (Goto et al., ACS Chem. Biol., 3, 120-129 (2008)). The relative yields of AR-VF-FLAG peptides initiated with other pre-charged fMetT derivatives were also comparable (FIG. 4D), indicating similar initiation efficiencies. When ValT was pre-charged with R-Phe, the yield of fMet-β-Phe-F-FLAG was 5-fold higher than the yield of fMet-VF-FLAG generated with pre-charged fMetT (FIG. 4E-4F). As initiation complex assembly is the rate limiting step during translation (Gualerzi & Pon, Cell. Mol. Life Sci., 72, 4341-4367 (2015)), the higher yield of fMet-β-Phe-F-FLAG relative to fMet-VF-FLAG is likely due to the difficulty assembling the translation initiation complex using non-natural fMetT derivatives. Benzoic acid monomers that were poor eFx substrates (yields <50%) in model MH reactions, such as 6 and 15 (FIG. 3A), failed to detectably initiate peptide synthesis from WT ribosomes in vitro. This observation indicates that the ribosome is largely agnostic of aramid structure, and that the concentration of non-natural fMetT derivative, rather than monomer structure, determines the reaction yield in PURExpress® reactions.

See also Table 2 below.

### Example 5: tRNA can be Charged with Substituted Malonate Derivatives, and Serve as a Substrate for Translation

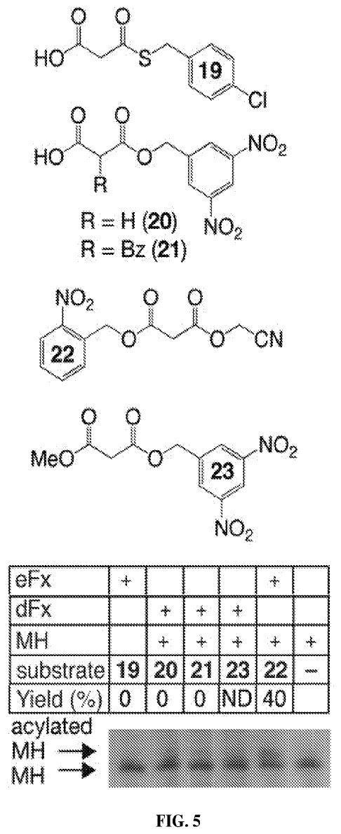

Like aramid natural products (Baumann et al., Angew. Chem. Int. Ed Engl., 53, 14605-14609 (2014)) polyketide-peptide hybrid molecules are believed to be biosynthesized by mega-assemblies of complex protein enzymes (Staunton & Weissman, Nat. Prod. Rep, 18, 380-416 (2001), Dutta et al., Nature, 510, 512-517 (2014), Robbins et al., Curr. Opin. Struct. Biol., 41, 10-18 (2016)), the combination of peptide and polyketide-based functionality can translate into highly unique biological functions (Du et al., Metab Eng, 3, 78-95 (2001), Silakowski et al., Chem. Biol., 8, 59-69 (2001), Walsh, Science, 303, 1805-1810 (2004)). To evaluate whether wild type E. coli ribosomes are capable of biosynthesizing a polyketide-peptide hybrid, malonate derivatives 19-23 (FIG. 5) were prepared. Model microhelix (MH) acylation reactions were analyzed using acid-urea gels (FIG. 5) and RNAse A/LC-HRMS as described above. Although the malonic acid half esters 19, 20, and 21 were poor substrates for the requisite Fx analog, cyanomethyl ester 22 was a moderate substrate, acylating the acylated MH in 40% yield. Although no gel-shift was observed in the eFx-promoted MH acylation reaction of cyanomethyl ester 23 (perhaps because of low molecular weight and/or polarity) (Fujino et al., ChemBioChem (2019)), strong evidence for reaction was observed using RNAse A/LC-HRMS. Indeed, addition of fMetT derivatives acylated with 22 and 23 (50-100 PM) to PURExpress® Δ (aa, tRNA) in vitro translation reactions led to the isolation of polypeptides carrying malonates 22 and 23 (22-VF-FLAG and 23-VF-FLAG, respectively), whose masses were confirmed by RNAse A/LC-HRMS. The yield of 23-VF-FLAG, estimated as described above was approximately 20% of the yield of fMet-VF-FLAG produced in reactions supplemented with pre-charged fMetT (FIG. 4A-4C). These results indicate that extant E. coli ribosomes have the capacity to biosynthesize simple polyketide-peptide hybrid molecules.

TABLE 2
Accurate mass characterization of aramid and malonyl
adenylates obtained by digestion microhelix charged
with various aramid and malonyl substrates.

| Adenylate | m/z (calc.) | m/z (obsv.) |
|---|---|---|
| 6 | 388.1364 | 388.1357 |
| 8 | 462.0831 | 462.0835 |
| 9 | 417.1153 | 417.1155 |
| 10 | 406.0913 | 406.0916 |
| 11 | 413.1316 | 413.1324 |
| 12 | 402.1408 | 402.1413 |
| 13 | 386.1459 | 386.1460 |
| 14 | 427.1473 | 427.1476 |
| 15 | 401.1568 | 401.1569 |
| 16 | 388.1252 | 388.1243 |
| 17 | 402.1408 | 402.1409 |
| 18 | 400.1252 | 400.1252 |
| 22 | 489.1365 | 489.1362 |
| 23 | 368.1201 | 368.1200 |

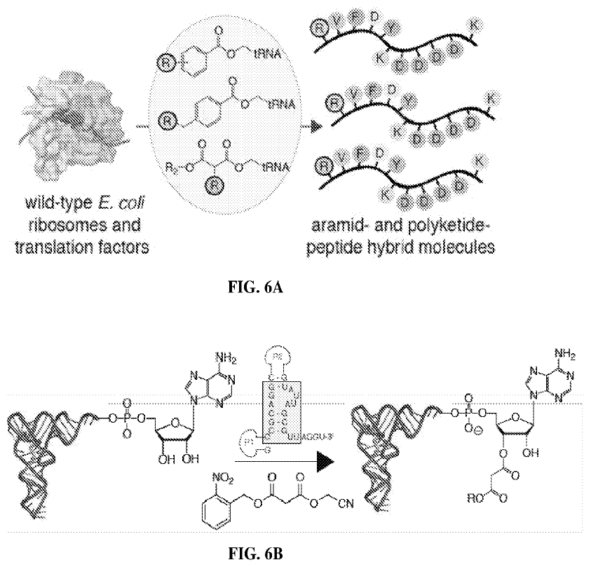

In summary, the foregoing experiment illustrate that wild type E. coli ribosomes accept pre-charged initiator tRNAs acylated with multiple substituted benzoic acids, including the monomeric unit of Kevlar, as well as malonyl (α,β-diketo) substrates. The ribosome then elongates these substrates to generate a diverse set of aramid-peptide and polyketide-peptide hybrid molecules (FIG. 6).

### Example 6: Exemplary Hybrid Polypeptides

### Materials and Methods

In vitro transcription/translation of short peptides containing a FLAG tag fMet-Val-Phe-Asp-Tyr-Lys-Asp-Asp-Asp-Asp-Lys (fMVF-Flag) (SEQ ID NO:14) was carried out using the PureExpress (ΔtRNA, Δaa (E6840S)) kit by New England Biolabs with the following modifications. To generate the fMVF-Flag WT peptide the following reactions were executed (25 μL): Solution A ((ΔtRNA, Δaa) (5 μL), 33 mM Methionine (0.25 μL), 33 mM Valine (0.25 L), solution containing 33 mM Tyrosine, 33 mM Phenylalanine, 33 mM Lysine (0.25 μL), 7 mM Asparatic acid (pH 7, 1 μL), tRNA solution (2.5 μL), Solution B (7.5 μL), 500-1000 ng dsDNA template (0.25-2 μL), and (water to 25 μL). When using precharged tRNA^{fMet}_{CAU} or tRNA^{Val}_{UAC}, either Valine or Methionine where omitted from the reaction mixture. The reactions were then incubated for 6 h.

### Results

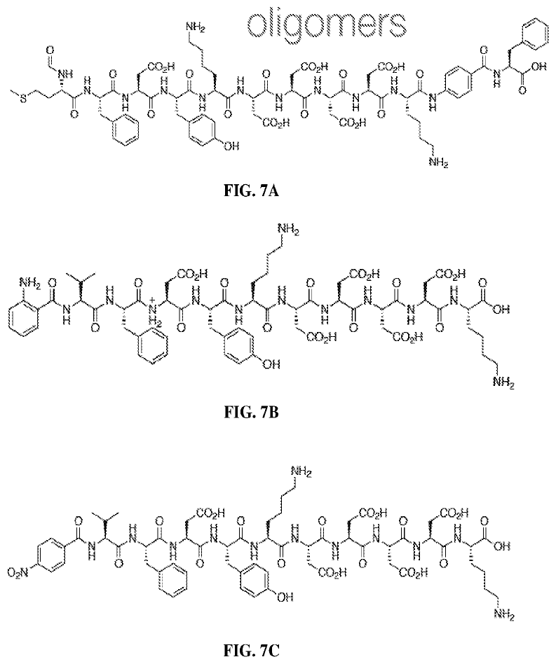

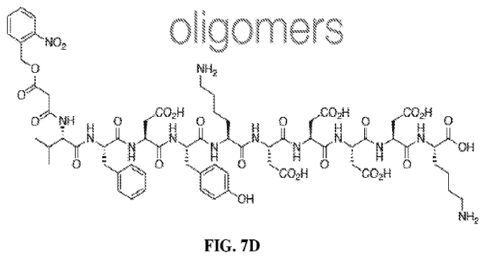

FIGS. 7A-7D are structures of oligomers prepared according to the disclosed methods.

FIG. 7A illustrates a hybrid aramid-peptide molecule formed when p-amino benzoic acid-Phe double monomer (para-aramid-Phe) is loaded into the A site of a ribosome and added to the C-terminal end of a growing polypeptide during translation. Mass Traces showed an Observed peak (M+2H): 793.3120 m/z relative to a Calculated (M+2H): 793.3112 m/z.

FIG. 7B illustrates a hybrid aramid-peptide molecule formed when an o-amino benzoic acid monomer (ortho-aramid) is loaded into the P site of a ribosome by an initiator tRNA and forms the N-terminus of a growing polypeptide during translation. Mass Traces showed an Observed peak (M+2H): 689.7940 m/z relative to a Calculated (M+2H): 689.7935 m/z.

FIG. 7C illustrates a hybrid aramid-peptide molecule formed when an p-nitro benzoic acid monomer (p-nitro aramid) is loaded into the P site of a ribosome by an initiator tRNA and forms the N-terminus of a growing polypeptide during translation. Mass Traces showed an Observed peak (M+2H): 704.7814 m/z relative to a Calculated (M+2H): 704.7812 m/z.

FIG. 7D illustrates a hybrid ketide-peptide molecule formed when a substituted malonic acid monomer is loaded into the P site of a ribosome by an initiator tRNA and forms the N-terminus of a growing polypeptide during translation. Mass Traces showed an Observed peak (M+2H): 740.7903 m/z relative to a Calculated (M+2H): 740.7912 m/z.

This result indicates that the ribosome is capable of biosynthesizing poly-keto-peptide hybrid molecules.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A functionalized tRNA comprising a functional molecule comprising or consisting of a benzoic acid or benzoic acid derivative acylated to the 3′ nucleotide of a natural or engineered tRNA or tRNA-like molecule.

2. The functionalized tRNA of claim 1 having a structure of Formula II, Formula II′, or Formula II″: 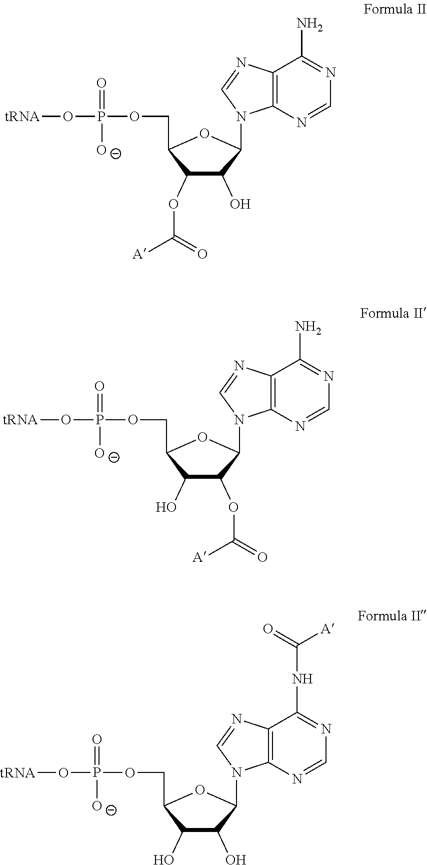
wherein A′ is an unsubstituted aryl group, a substituted aryl group, an unsubstituted heteroaryl group, or a substituted heteroaryl group,
the adenine of Formula II, Formula II′, or Formula II″ is the 3′ nucleotide of the tRNA, and the adenine can be adenine, cytosine, guanine, or uracil in Formula II or Formula II′, or adenine, cytosine, or guanine in Formula II″, and
the “tRNA” of Formula II, Formula II′, or Formula II″ comprises the remaining nucleotides of the functionalized tRNA.

3.-5. (canceled)

6. The functionalized tRNA of claim 1 having a structure of Formula III, Formula III′, or Formula III″: 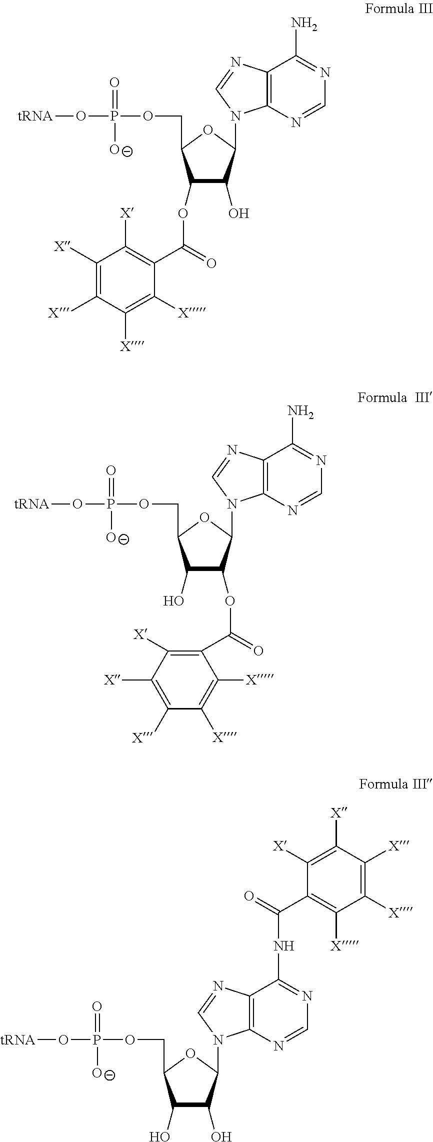
wherein X′, X″, X′″, X″″, and X′″″ are independently a hydrogen atom, a deuterium atom, a tritium atom, or a halogen atom selected from fluorine, chlorine, bromine, and iodine,
the adenine of Formula III, Formula III′, or Formula III″ is the 3′ nucleotide of the tRNA, and the adenine can be adenine, cytosine, guanine, or uracil in Formula III or Formula III′, or adenine, cytosine, or guanine in Formula III″, and
the “tRNA” of Formula III, Formula III′, or Formula III″ comprises the remaining nucleotides of the functionalized tRNA.

7. (canceled)

8. The functionalized tRNA of claim 1 having a structure of Formula IV, Formula IV′, or Formula IV″: 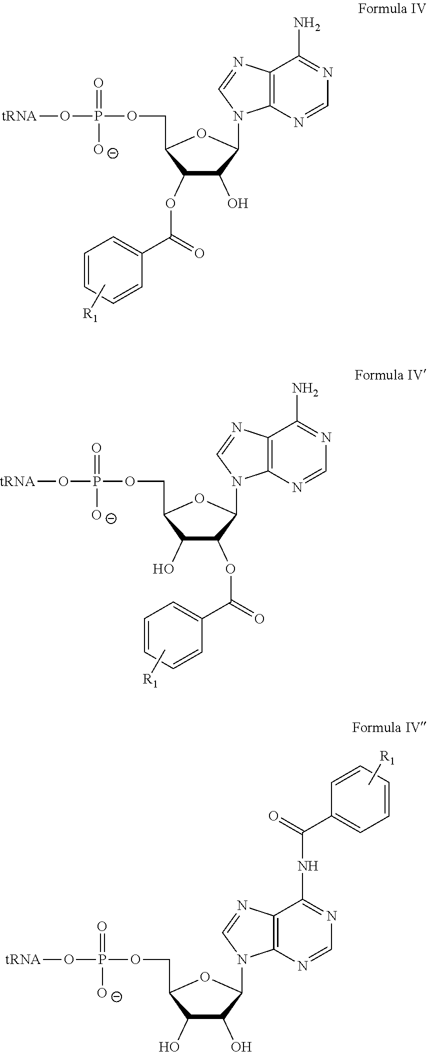
wherein R₁ is
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;
an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or
a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,
the adenine of Formula IV, Formula IV′, or Formula IV″ is the 3′ nucleotide of the tRNA, and the adenine can be adenine, cytosine, guanine, or uracil in Formula IV or Formula IV′, or adenine, cytosine, or guanine in Formula IV″, and
the “tRNA” of Formula IV, Formula IV′, or Formula IV″ comprises the remaining nucleotides of the functionalized tRNA.

9.-11. (canceled)

12. The functionalized tRNA of claim 1, having a structure of Formula V, Formula V′, or Formula V″: 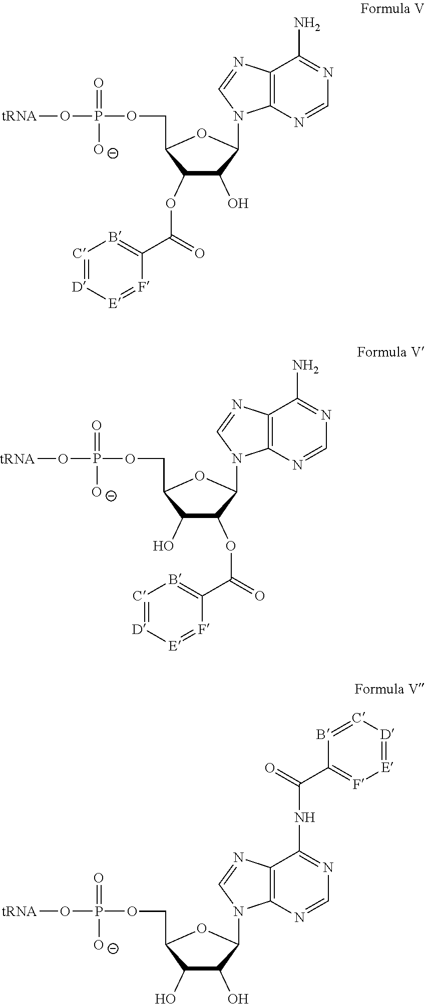
where B′, C′, D′, E′, and F′ are independently C—R₁ or a nitrogen atom;
where at least one of B′, C′, D′, E′, and F′ is a nitrogen atom;
wherein R₁ is
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;
an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or
a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,
the adenine of Formula V, Formula V′, or Formula V″ is the 3′ nucleotide of the tRNA, and the adenine can be adenine, cytosine, guanine, or uracil in Formula V or Formula V′, or adenine, cytosine, or guanine in Formula V″, and
the “tRNA” of Formula V, Formula V′, or Formula V″ comprises the remaining nucleotides of the functionalized tRNA.

13. A functionalized tRNA comprising a functional molecule comprising or consisting of a malonic acid or malonic acid derivative acylated to the 3′ nucleotide of a natural or engineered tRNA or tRNA-like molecule.

14. The functionalized tRNA of claim 13 having a structure of Formula XIV, XIV′, or XIV″: 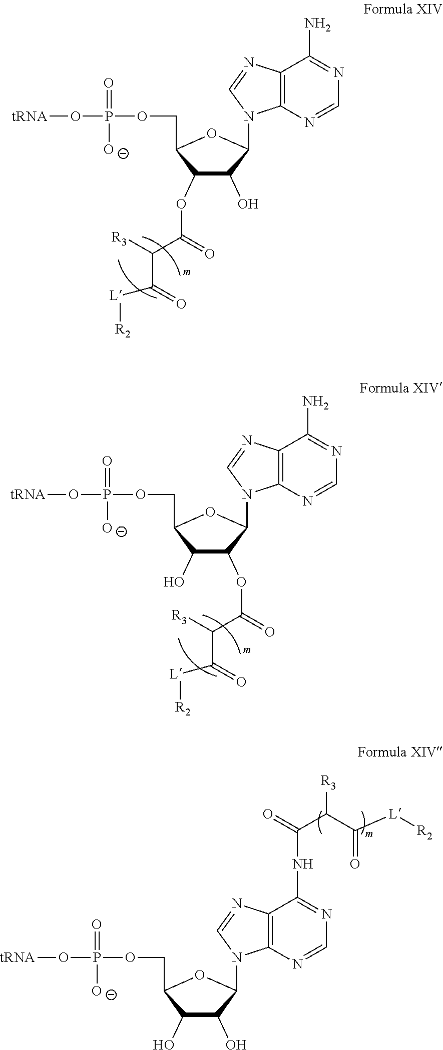
(a) wherein L′ is an oxygen atom, a nitrogen atom, or a sulfur atom;
(b) wherein m is an integer between 1 and 10 inclusive; and
(c) wherein R₂ and each R₃ are independently:
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;
an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or
a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof,
the adenine of Formula XIV, Formula XIV′, or Formula XIV″ is the 3′ nucleotide of the tRNA, and the adenine can be adenine, cytosine, guanine, or uracil in Formula XIV or Formula XIV′, or adenine, cytosine, or guanine in Formula XIV″, and
the “tRNA” of Formula XIV, Formula XIV′, or Formula XIV″ comprises the remaining nucleotides of the functionalized tRNA.

15.-17. (canceled)

18. The functionalized tRNA of claim 1 having a structure of Formula XII: 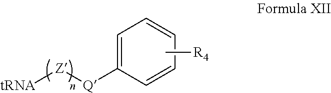
(a) wherein each Z′ is an amino acid;
(b) wherein n is an integer between 1 and 4 inclusive;
(c) wherein Q′ is an amide group or an ester group; and
(d) wherein R₄ comprises an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

19. (canceled)

20. The functionalized tRNA of claim 1, wherein the tRNA is an initiator tRNA.

21. The functionalized tRNA of claim 1, wherein the tRNA is an elongator tRNA.

22.-25. (canceled)

26. A method of making a functionalized polypeptide comprising providing or expressing a messenger RNA (mRNA) encoding the target polypeptide in a translation system comprising
the functionalized tRNA of claim 1,
wherein the functionalized tRNA recognizes at least one codon such that functional molecule is incorporated into a polypeptide during translation.

27.-31. (canceled)

32. A functionalized polypeptide comprising two or more amino acids and at least one functional molecule comprising or consisting of a benzoic acid or benzoic acid derivative; or a malonic acid or malonic acid derivative.

33. The functionalized polypeptide of claim 32 comprising the functional molecule at the N-terminus, the C-terminus, internally or a combination thereof.

34.-35. (canceled)

36. The functionalized polypeptide of claim 32 having a structure of Formula VI: 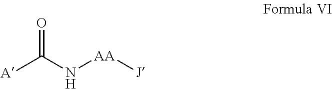
wherein A′ is an unsubstituted aryl group, a substituted aryl group, an unsubstituted heteroaryl group, or a substituted heteroaryl group;
wherein NH-AA is an amino acid which is linked to the functional molecule through a peptide bond; and
wherein J′ is one or more amino acids.

37.-38. (canceled)

39. The functionalized polypeptide of claim 36 having a structure of Formula VII: 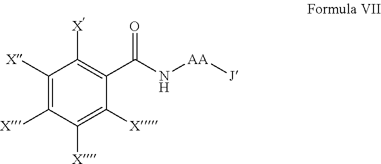
wherein NH-AA is an amino acid which is linked to the functional molecule through a peptide bond;
wherein J′ is one or more amino acids; and
wherein X′, X″, X′″, X″″, and X′″″ are independently a hydrogen atom, a deuterium atom, a tritium atom, or a halogen atom selected from fluorine, chlorine, bromine, and iodine.

40. (canceled)

41. The functionalized polypeptide of claim 36 having a structure of Formula VIII: 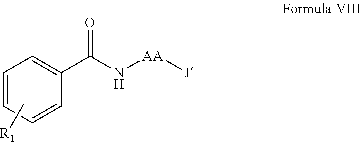
wherein NH-AA is an amino acid which is linked to the functional molecule through a peptide bond;
wherein J′ is one or more amino acids; and
wherein R₁ is
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;
an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or
a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

42.-44. (canceled)

45. The functionalized polypeptide of claim 36 having a structure of Formula IX: 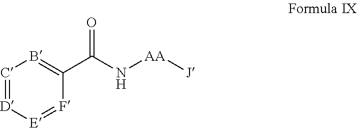
wherein NH-AA is an amino acid which is linked to the functional molecule through a peptide bond;
wherein J′ is one or more amino acids;
where B′, C′, D′, E′, and F′ are independently C—R₁ or a nitrogen atom;
where at least one of B′, C′, D′, E′, and F′ is a nitrogen atom; and
wherein R₁ is
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;
an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or
a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

46. The functionalized polypeptide of claim 32 having a structure of Formula XI: 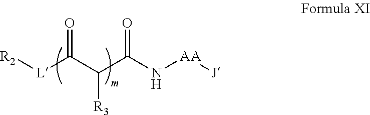
(a) wherein L′ is an oxygen atom, a nitrogen atom, or a sulfur atom;
(b) wherein m is an integer between 1 and 10 inclusive; and
(c) wherein R₂ and each R₃ are independently:
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;
an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;
an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;
an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or
a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

47.-48. (canceled)

49. The functionalized polypeptide of claim 32 having a structure of Formula XII′: 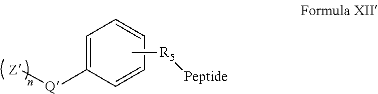
(a) wherein each Z′ is an amino acid;
(b) wherein n is an integer between 1 and 4 inclusive;
(c) wherein Q′ is an amide group or an ester group; and
(d) wherein R₅ comprises a secondary amino group optionally containing a substituent at the amino nitrogen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group.

50. A functionalized polypeptide made according to the method of claim 26.

51. (canceled)


SEQUENCE LISTING (18 sequences)

SEQ ID NO: 1 - RNA, 45 nt, Artificial Sequence
  synthetic polynucleotide
ggaucgaaagauuuccgcauccccgaaaggguacauggcguuagg

SEQ ID NO: 2 - RNA, 45 nt, Artificial Sequence
  synthetic polynucleotide
ggaucgaaagauuuccgcggccccgaaaggggauuagcguuaggu

SEQ ID NO: 3 - RNA, 47 nt, Artificial Sequence
  synthetic polynucleotide
ggaucgaaagauuuccgcacccccgaaagggguaaguggcguuaggu

SEQ ID NO: 4 - DNA, 79 bp, Artificial Sequence
  synthetic polynucleotide
aattcctgcagtaatacgactcactatagggtgattagctcagctgggagagcacctcccttacaaggagggggtcggc

SEQ ID NO: 5 - DNA, 79 bp, Artificial Sequence
  synthetic polynucleotide
aattcctgcagtaatacgactcactatagggtgattagctcagctgggagagcacctccctctaaaggagggggtcggc

SEQ ID NO: 6 - DNA, 39 bp, Artificial Sequence
  synthetic polynucleotide
  misc_feature (1)..(1): n = 2-O-methyl-deoxymethylguanosine
ngtgggtgatgacgggatcgaaccgccgaccccctcctt

SEQ ID NO: 7 - DNA, 66 bp, Artificial Sequence
  synthetic polynucleotide
aattcctgcagtaatacgactcactatacgcggggtggagcagcctggtagctcgtcgggctcata

SEQ ID NO: 8 - DNA, 56 bp, Artificial Sequence
  synthetic polynucleotide
  misc_feature (1)..(1): n = 2-O-methyl-deoxymethylguanosine
ngttgcgggggccggatttgaaccgacgaccttcgggttatgagcccgacgagcta

SEQ ID NO: 9 - DNA, 62 bp, Artificial Sequence
  synthetic polynucleotide
taatacgactcactatagggttaactttaacaaggagaaaaacatggtatttgactacaagg

SEQ ID NO: 10 - DNA, 61 bp, Artificial Sequence
  synthetic polynucleotide
cgaagcttacttgtcgtcgtcgtccttgtagtcaaataccatgtttttctccttgttaaag

SEQ ID NO: 11 - DNA, 38 bp, Artificial Sequence
  synthetic polynucleotide
gcgaattaatacgactcactatagggttaactttaaca

SEQ ID NO: 12 - DNA, 54 bp, Artificial Sequence
  synthetic polynucleotide
aaacccctccgtttagagaggggttatgctagttacttgtcgtcgtcgtccttg

SEQ ID NO: 13 - RNA, 22 nt, Artificial Sequence
  synthetic polynucleotide
ggcucuguucgcagagccgcca

SEQ ID NO: 14 - protein, 11 aa, Artificial Sequence
  synthetic polypeptide
MVFDYKDDDDK

SEQ ID NO: 15 - protein, 11 aa, Artificial Sequence
  synthetic polypeptide
  MISC_FEATURE (1)..(1): FORMYLATION
MVFDYKDDDDK

SEQ ID NO: 16 - protein, 10 aa, Artificial Sequence
  synthetic polypeptide
  MISC_FEATURE (1)..(1): N-terminal o- or m- aminobenzoic acid moiety
VFDYKDDDDK

SEQ ID NO: 17 - protein, 10 aa, Artificial Sequence
  synthetic polypeptide
  MISC_FEATURE (1)..(1): N-terminal aramid or polyketide moiety
VFDYKDDDDK

SEQ ID NO: 18 - RNA, 15 nt, Artificial Sequence
  synthetic polynucleotide
guauuagcguuaggu